(19) United States
(12) Patent Application Publication
(10) Pub. No.: US 2015/0266878 A1
(43) Pub. Date: Sep. 24, 2015

(54) NOVEL BI-RING PHENYL-PYRIDINES/PYRAZINES FOR THE TREATMENT OF CANCER

(71) Applicant: HOFFMANN-LA ROCHE INC., Little Falls, NJ (US)

(72) Inventors: Song Yang, Shanghai (CN); Zhanling Cheng, Shanghai (CN); Chengang Zhou, Shanghai (CN); Min Jiang, Shanghai (CN); Jianhua Wang, Shanghai (CN); Min Wang, Shanghai (CN); Xingchun Han, Shanghai (CN)

(73) Assignee: HOFFMANN-LA ROCHE INC., Little Falls, NJ (US)

(21) Appl. No.: 14/735,348

(22) Filed: Jun. 10, 2015

Related U.S. Application Data
(63) Continuation of application No. PCT/EP2013/075751, filed on Dec. 6, 2013.
Application No. PCT/EP2013/075751 is a continuation of application No. PCT/CN2012/086275, filed on Dec. 10, 2012.

(51) Int. Cl. C07D 471/04; C07D 413/04; C07F 5/04; C07D 405/04; C07F 5/02; C07D 213/74; C07D 417/04; C07D 401/04

(52) U.S. Cl. CPC C07D 471/04; C07D 417/04; C07D 413/04; C07D 401/04; C07D 405/04; C07F 5/027; C07D 213/74; C07F 5/04

(57) ABSTRACT

The invention provides novel compounds having the general formula:

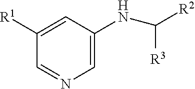

wherein R¹, R² and R³ are as described herein, compositions including the compounds and methods of using the compounds.

### CROSS REFERENCE TO RELATED APPLICATIONS

This application is a continuation of International Application No. PCT/EP2013/075751 having an international filing date of Dec. 6, 2013, the entire contents of which are incorporated herein by reference, and which claims benefit under 35 U.S.C. §119 to International Application No. PCT/CN2012/086275, filed Dec. 10, 2012, the entire contents of which are incorporated herein by reference.

The present invention relates to organic compounds useful for therapy in a mammal, and in particular to inhibit cell proliferation and induce cell cycle arrest and apoptosis that overexpress CDK8 or Cyclin C useful for treating cancer.

### FIELD OF THE INVENTION

The cyclin-dependent kinase (CDK) complexes are well-conserved Ser/Thr kinase family, and it has been shown to be activated by the binding of regulatory partner, generally a cyclin. There are total 20 CDK family members and 5 CDK-like proteins based on the similarities in sequence and function. CDKs regulate various key transitions of cell cycle and play an important role in the regulation of transcription, apoptosis and neuronal functions.

Dysregulation of CDKs has been linked to pathological events and both proliferative and non-proliferative disease, including cancers, Alzhemers disease (AD), parkinson's disease, Stroke/ischemia, pain, traumatic brain injury, kidney disease, inflammation pathologies, type 2 diabetes, viral infection (HSV, HCMV, HPV, HIV).

CDK8 is a CyclinC-dependent CDK family kinase and functions as a transcriptional regulator. Several phosphorylation targets of CDK8 have been identified, including the RNA polymerase II (RNAPII) C-terminal domain (CTD), histone H3, subunits of general transcription factors (GTFs) and certain transactivators. CDK8 has also been described as a transcriptional coactivator in oncongenic signaling pathways, including the β-catenin pathway, the serum response network, the Tumor Growth Factor TGFβ signaling pathway, the p53 pathway, as well as in thyroid hormone-dependent transcription. Colocalization of CDK8 and Cyclin C was also reported in neurodegenerative disease such as AD. CDK8 was found to be frequently dysregulated in various human cancers, such as colon cancer, gastric cancer and melanoma. Inhibition of CDK8 by short hairpin RNA (shRNA) inhibits cancer cell proliferation, and induces cell cycle arrest and apoptosis in vitro and in vivo models. Although Silibinin, the major active constituent of silymarin isolated from milk thistle (Silybum marianum), has shown strong cell growth inhibition in colon cancer through downregulation CDK8 expression, there are no known direct CDK8 inhibitors under clinical development. Therefore, there is a great unmet medical need to develop CDK8 inhibitors for cancer patients.

### SUMMARY OF THE INVENTION

Objects of the present invention are novel compounds of formula I, their manufacture, medicaments based on a compound in accordance with the invention and their production as well as the use of compounds of formula I for the treatment of cancer.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 1 to 4 carbon atoms, for example methyl, ethyl, propyl, isopropyl, 1-butyl, 2-butyl, tert-butyl and the like. Particular “C₁₋₆alkyl” groups are methyl, ethyl, isopropyl and tert-butyl.

The term “C₁₋₆alkoxy” alone or in combination signifies a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy and the like. Particular “C₁₋₆alkoxy” groups are methoxy and ethoxy and more particularly methoxy.

The term “CₓH₂ₓ” alone or in combination signifies a saturated, linear- or branched chain alkyl group containing 1 to 6, particularly 1 to 4 carbon atoms.

The term “C_{y}H_{2y}” alone or in combination signifies a saturated, linear- or branched chain alkyl group containing 2 to 6, particularly 2 to 4 carbon atoms.

The term “cycloalkyl”, alone or in combination, refers to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “cycloalkyl” groups are cyclopropyl, cyclopentyl and cyclohexyl.

The term “amino”, alone or in combination, refers to primary (—NH₂), secondary (—NH—) or tertiary amino (

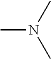

).

The term “halogen” means fluorine, chlorine, bromine or iodine. Halogen is particularly fluorine or chlorine.

The term “hydroxy” alone or in combination refers to the group —OH.

The term “carbonyl” alone or in combination refers to the group —C(O)—.

The term “sulfanyl” alone or in combination refers to the group —S—.

The term “sulfonyl” alone or in combination refers to the group —S(O)₂—.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula I and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435; or in Ansel, H., et al., In: Pharmaceutical Dosage Forms and Drug Delivery Systems, 6th ed. (1995), pp. 196 and 1456-1457. Particular are the sodium salts of the compounds of formula I.

Compounds of the general formula I which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

### Inhibitors of CDK8 Or Cyclin C

The present invention provides (i) novel compounds having the general formula I:

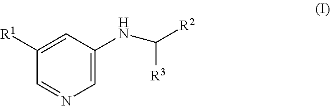

wherein

R¹ is selected from

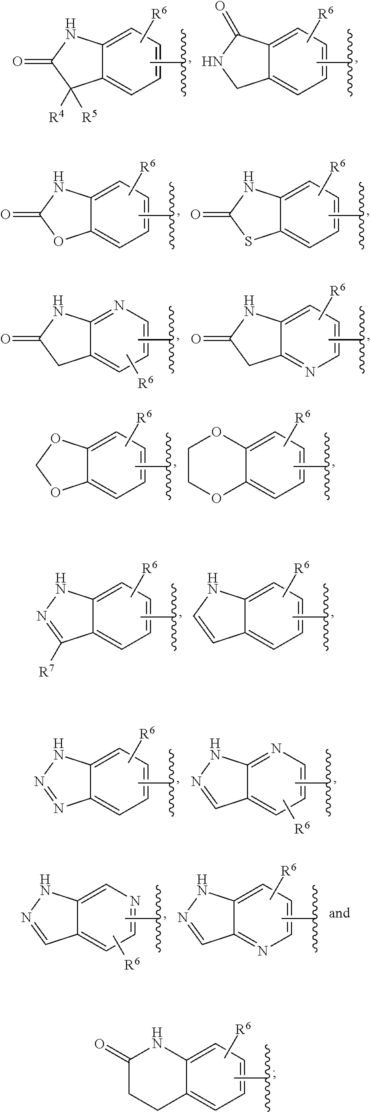

R² is aminocarbonyl, C₁₋₆alkoxy-C_{y}H_{2y}-amino-CₓH₂ₓ—, C₁₋₆alkoxy-CₓH₂ₓ-sulfonylamino-CₓH₂ₓ—, C₁₋₆alkylcarbonylamino-CₓH₂ₓ—, C₁₋₆ alkylsulfonylamino-CₓH₂ₓ—, cycloalkylcarbonylamino-CₓH₂ₓ—, cycloalkylsulfonylamino-CₓH₂ₓ—, hydroxy-CₓH₂ₓ—, hydroxy-C_{y}H_{2y}-amino-CₓH₂ₓ—, hydroxy-CₓH₂ₓ-carbonylamino-CₓH₂ₓ— or phenylcarbonylamino-CₓH₂ₓ—;

R³ is phenyl, which is unsubstituted or substituted by halogen;

R⁴ is hydrogen, C₁₋₆alkyl or halogen;

R⁵ is hydrogen, C₁₋₆alkyl or halogen;

or R⁴ and R⁵, together with the carbon atom, to which they are attached, form cycloalkyl;

R⁶ is hydrogen or halogen;

R⁷ is hydrogen, C₁₋₆alkyl, C₁₋₆alkylsulfanyl, C₁₋₆alkylsulfonyl, amino or halogen;

x is 1-6;

y is 2-6;

or pharmaceutically acceptable salt thereof.

Another embodiment of present invention is (ii) a compound of formula I, wherein

R¹ is selected from

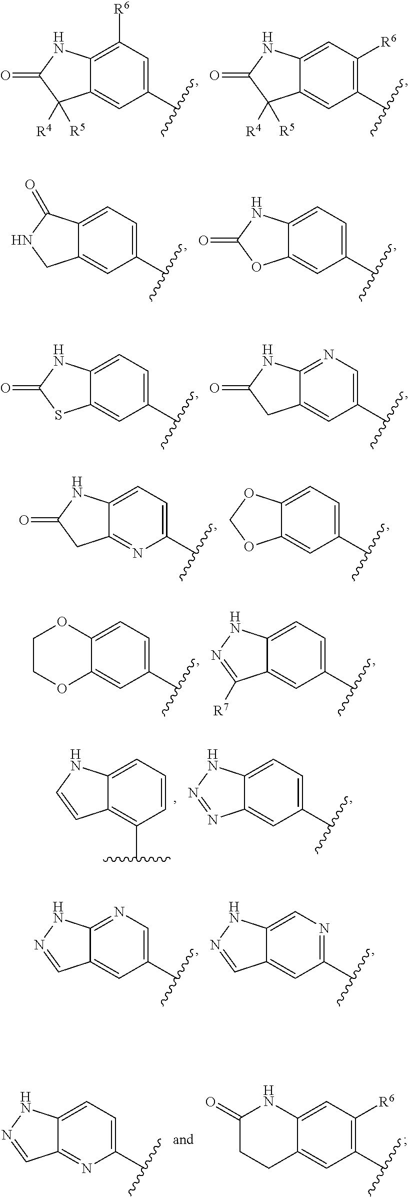

R² is aminocarbonyl, C₁₋₆alkoxy-C_{y}H_{2y}-amino-CₓH₂ₓ—, C₁₋₆alkoxy-CₓH₂ₓ-sulfonylamino-CₓH₂ₓ—, C₁₋₆alkylcarbonylamino-CₓH₂ₓ—, C₁₋₆alkylsulfonylamino-CₓH₂ₓ—, cycloalkylcarbonylamino-CₓH₂ₓ—, cycloalkylsulfonylamino-CₓH₂ₓ—, hydroxy-CₓH₂ₓ—, hydroxy-C_{y}H_{2y}-amino-CₓH₂ₓ—, hydroxy-CₓH₂ₓ-carbonylamino-CₓH₂ₓ— or phenylcarbonylamino-CₓH₂ₓ—;

R³ is phenyl, which is unsubstituted or once substituted by halogen;

R⁴ is hydrogen, C₁₋₆alkyl or halogen;

R⁵ is hydrogen, C₁₋₆alkyl or halogen;

or R⁴ and R⁵, together with the carbon atom, to which they are attached, form cycloalkyl;

R⁶ is hydrogen or halogen;

R⁷ is hydrogen, C₁₋₆alkyl, C₁₋₆alkylsulfanyl, C₁₋₆alkylsulfonyl, amino or halogen;

x is 1-6;

y is 2-6;

or pharmaceutically acceptable salt thereof.

Further embodiment of present invention is (iii) a compound of formula I, wherein

R¹ is selected from

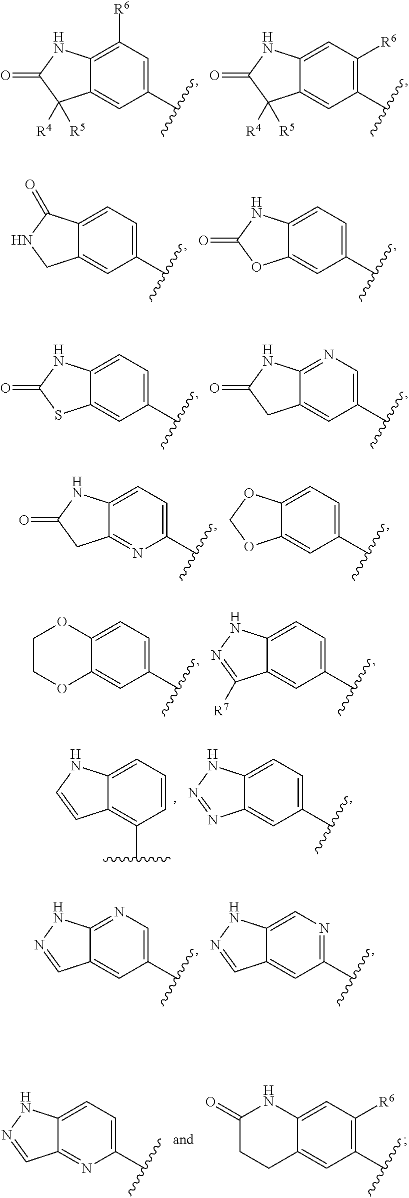

R² is aminocarbonyl, methoxyethylaminomethyl, methoxyethylsulfonylaminomethyl, methylcarbonylaminomethyl, ethylcarbonylaminomethyl, isopropylcarbonylaminomethyl, methylsulfonylaminomethyl, cyclohexylcarbonylaminomethyl, cyclopropylsulfonylaminomethyl, hydroxymethyl, hydroxyethylaminomethyl, hydroxymethylcarbonylaminomethyl or phenylcarbonylaminomethyl;

R³ is phenyl or chlorophenyl;

R⁴ is hydrogen, methyl or fluoro;

R⁵ is hydrogen, methyl or fluoro;

or R⁴ and R⁵, together with the carbon atom, to which they are attached, form cyclopropyl;

R⁶ is hydrogen or fluoro;

R⁷ is hydrogen, methyl, ethyl, methylsulfanyl, methylsulfonyl, amino, fluoro or chloro;

or pharmaceutically acceptable salt thereof.

Another embodiment of present invention is (iv) a compound of formula I or a pharmaceutically acceptable salt thereof, wherein

R¹ is

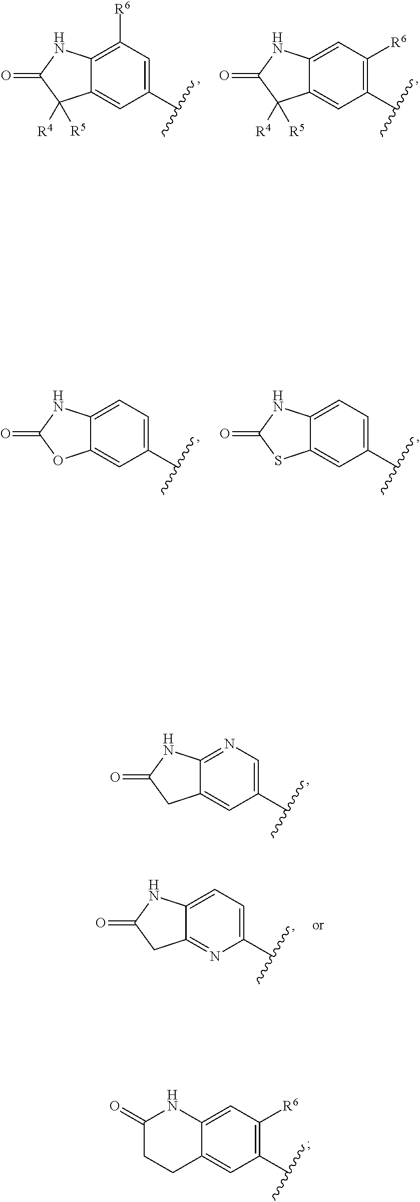

R² is aminocarbonyl, C₁₋₆alkylcarbonylamino-CₓH₂ₓ— or hydroxy-CₓH₂ₓ—;

R³ is phenyl, which is unsubstituted or once substituted by halogen;

R⁴ is hydrogen, C₁₋₆alkyl or halogen;

R⁵ is hydrogen, C₁₋₆alkyl or halogen;

or R⁴ and R⁵, together with the carbon atom, to which they are attached, form cycloalkyl;

R⁶ is hydrogen or halogen;

x is 1-6.

Further embodiment of present invention is (v) a compound of formula I or a pharmaceutically acceptable salt thereof, wherein

R¹ is

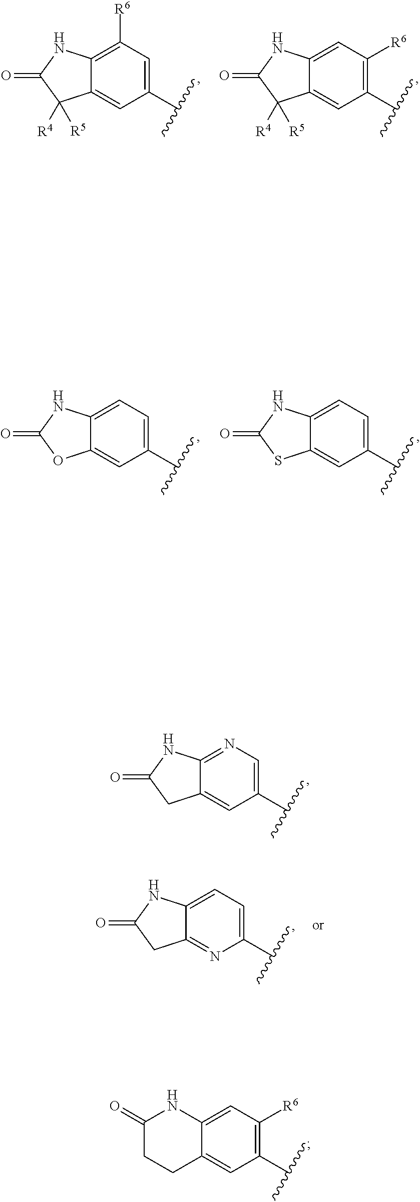

R² is aminocarbonyl, methylcarbonylaminomethyl or hydroxymethyl;

R³ is phenyl or chlorophenyl;

R⁴ is hydrogen, methyl or fluoro;

R⁵ is hydrogen, methyl or fluoro;

or R⁴ and R⁵, together with the carbon atom, to which they are attached, form cyclopropyl;

R⁶ is hydrogen or fluoro.

Another embodiment of present invention is (vi) a compound of formula I or a pharmaceutically acceptable salt thereof, wherein

R¹ is

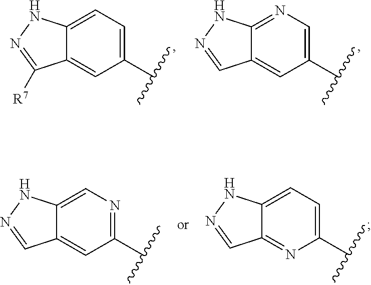

R² is aminocarbonyl, C₁₋₆alkoxy-C_{y}H_{2y}-amino-CₓH₂ₓ—, C₁₋₆alkoxy-CₓH₂ₓ-sulfonylamino-CₓH₂ₓ—, C₁₋₆alkylcarbonylamino-CₓH₂ₓ—, C₁₋₆alkylsulfonylamino-CₓH₂ₓ—, cycloalkylcarbonylamino-CₓH₂ₓ—, cycloalkylsulfonylamino-CₓH₂ₓ—, hydroxy-CₓH₂ₓ—, hydroxy-C_{y}H_{2y}-amino-CₓH₂ₓ—, hydroxy-CₓH₂ₓ-carbonylamino-CₓH₂ₓ— or phenylcarbonylamino-CₓH₂ₓ—;

R³ is phenyl;

R⁷ is hydrogen, C₁₋₆alkyl, C₁₋₆alkylsulfanyl, C₁₋₆alkylsulfonyl, amino or halogen;

x is 1-6;

y is 2-6.

Further embodiment of present invention is (vii) a compound of formula I or a pharmaceutically acceptable salt thereof, wherein

R¹ is

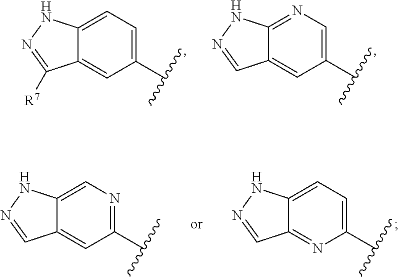

R² is aminocarbonyl, methoxyethylaminomethyl, methoxyethylsulfonylaminomethyl, methylcarbonylaminomethyl, ethylcarbonylaminomethyl, isopropylcarbonylaminomethyl, methylsulfonylaminomethyl, cyclohexylcarbonylaminomethyl, cyclopropylsulfonylaminomethyl, hydroxymethyl, hydroxyethylaminomethyl, hydroxymethylcarbonylaminomethyl or phenylcarbonylaminomethyl;

R³ is phenyl;

R⁷ is hydrogen, methyl, ethyl, methylsulfanyl, methylsulfonyl, amino, fluoro or chloro.

Another embodiment of present invention is (viii) a compound of formula I or a pharmaceutically acceptable salt thereof, wherein

R¹ is

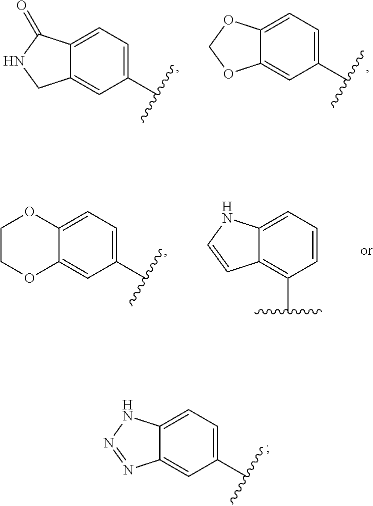 [SMILES: *c1ccc2c(c1)CNC2=O ; *c1ccc2c(c1)OCO2 ; *c1ccc2c(c1)OCCO2 ; *c1cccc2[nH]ccc12 ; *c1ccc2[nH]nnc2c1]

R² is aminocarbonyl or hydroxy-CₓH₂ₓ—;

R³ is phenyl;

x is 1-6.

Further embodiment of present invention is (ix) a compound of formula I or a pharmaceutically acceptable salt thereof, wherein

R¹ is

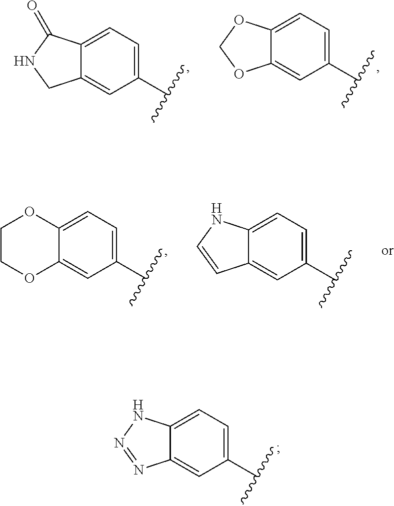 [SMILES: *c1ccc2c(c1)CNC2=O ; *c1ccc2c(c1)OCO2 ; *c1ccc2c(c1)OCCO2 ; *c1ccc2[nH]ccc2c1 ; *c1ccc2[nH]nnc2c1]

R² is aminocarbonyl or hydroxymethyl;

R³ is phenyl.

Particular compounds of formula I, including their activity data, NMR data and MS data are summarized in the following Table 1, 2 and 3.

TABLE 1
Structure, name and activity data of particular compounds

| Example No. | Structure | Name | IC₅₀ (μM) |
|---|---|---|---|
| 1 | 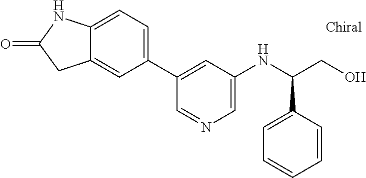 | 5-[5-((R)-2-Hydroxy-1- phenyl-ethylamino)- pyridin-3-yl]-1,3-dihydro- indol-2-one | 0.0035 |
| 2 | 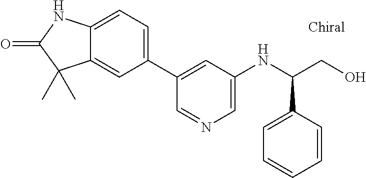 | 5-[5-((R)-2-Hydroxy-1- phenyl-ethylamino)- pyridin-3-yl]-3,3- dimethyl-1,3-dihydro- indol-2-one | 0.132 |
| 3 | 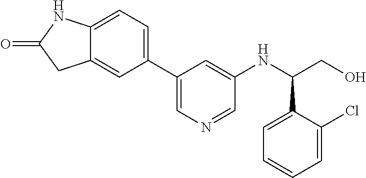 [SMILES: O=C1Cc2cc(-c3cncc(N[C@@H](CO)c4ccccc4Cl)c3)ccc2N1] | 5-{5-[(R)-1-(2-Chloro- phenyl)-2-hydroxy- ethylamino]-pyridin-3- yl}-1,3-dihydro-indol-2- one | 0.0021 |
| 4 | 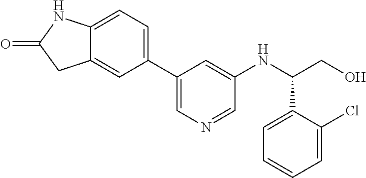 [SMILES: O=C1Cc2cc(-c3cncc(N[C@H](CO)c4ccccc4Cl)c3)ccc2N1] | 5-{5-[(S)-1-(2-Chloro- phenyl)-2-hydroxy- ethylamino]-pyridin-3- yl}-1,3-dihydro-indol-2- one | 0.157 |
| 5 | 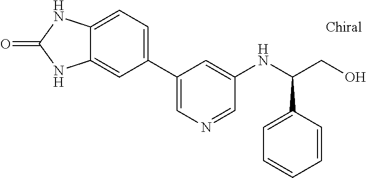 | 5-[5-((R)-2-Hydroxy-1- phenyl-ethylamino)- pyridin-3-yl]-1,3-dihydro- benzoimidazol-2-one | 0.0011 |
| 6 | 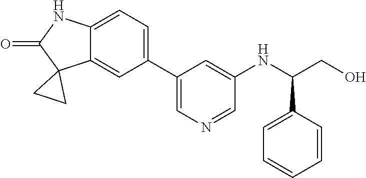 [SMILES: O=C1Nc2ccc(-c3cncc(N[C@@H](CO)c4ccccc4)c3)cc2C12CC2] | (R)-5′-(5-((2-Hydroxy-1- phenylethyl)amino)pyridin- 3-yl)-spiro[cyclopropane- 1,3′-indolin]-2′-one | 0.0029 |
| 7 | 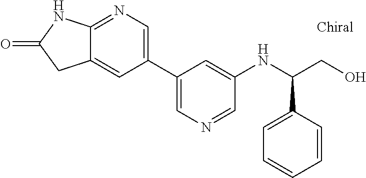 | 5-[5-((R)-2-Hydroxy-1- phenyl-ethylamino)- pyridin-3-yl]-1,3-dihydro- pyrrolo[2,3-b]pyridin-2- one | 0.315 |
| 8 | 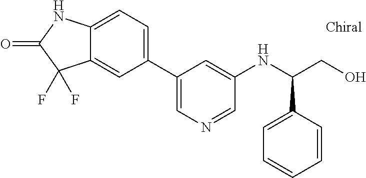 | 3,3-Difluoro-5-[5-((R)-2- hydroxy-1-phenyl- ethylamino)-pyridin-3- yl]-1,3-dihydro-indol-2- one | 0.0376 |
| 9 | 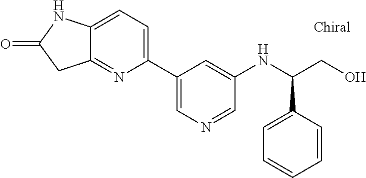 | 5-[5-((R)-2-Hydroxy-1- phenyl-ethylamino)- pyridin-3-yl]-1,3-dihydro- pyrrolo[3,2-b]pyridin-2- one | 0.0813 |
| 10 | 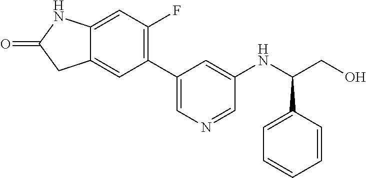 [SMILES: O=C1Cc2cc(-c3cncc(N[C@@H](CO)c4ccccc4)c3)c(F)cc2N1] | 6-Fluoro-5-[5-((R)-2- hydroxy-1-phenyl- ethylamino)-pyridin-3- yl]-1,3-dihydro-indol-2- one | 0.0005 |
| 11 | 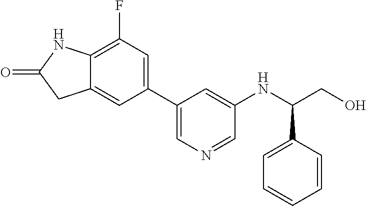 [SMILES: O=C1Cc2cc(-c3cncc(N[C@@H](CO)c4ccccc4)c3)cc(F)c2N1] | 7-Fluoro-5-[5-((R)-2- hydroxy-1-phenyl- ethylamino)-pyridin-3- yl]-1,3-dihydro-indol-2- one | 0.0278 |
| 12 | 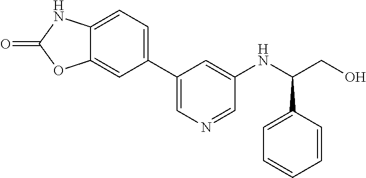 [SMILES: O=c1[nH]c2ccc(-c3cncc(N[C@@H](CO)c4ccccc4)c3)cc2o1] | 6-[5-((R)-2-Hydroxy-1- phenyl-ethylamino)- pyridin-3-yl]-3H- benzooxazol-2-one | 0.0038 |
| 13 | 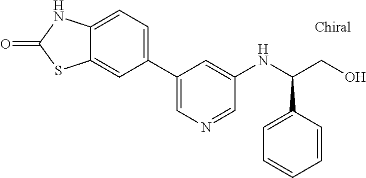 | 6-[5-((R)-2-Hydroxy-1- phenyl-ethylamino)- pyridin-3-yl]-3H- benzothiazol-2-one | 0.0078 |
| 14 | 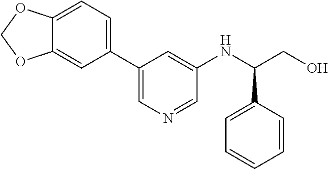 [SMILES: OC[C@H](Nc1cncc(-c2ccc3c(c2)OCO3)c1)c1ccccc1] | (R)-2-(5-Benzo[1,3]dioxol- 5-yl-pyridin-3-ylamino)-2- phenyl-ethanol | 0.0463 |
| 15 | 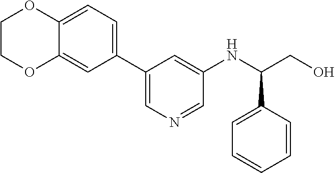 [SMILES: OC[C@H](Nc1cncc(-c2ccc3c(c2)OCCO3)c1)c1ccccc1] | (R)-2-[5-(2,3-Dihydro- benzo[1,4]dioxin-6-yl)- pyridin-3-ylamino]-2- phenyl-ethanol | 0.35 |
| 16 | 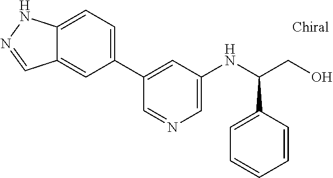 | (R)-2-[5-(1H-Indazol-5- yl)-pyridin-3-ylamino]-2- phenyl-ethanol | 0.0002 |
| 17 | 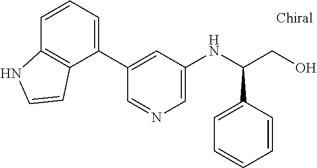 | (R)-2-[5-(1H-Indol-4-yl)- pyridin-3-ylamino]-2- phenyl-ethanol | 0.1911 |
| 18 | 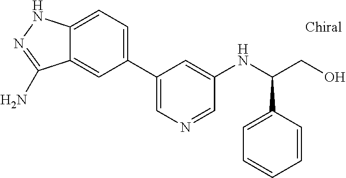 | (R)-2-[5-(3-Amino-1H- indazol-5-yl)-pyridin-3- ylamino]-2-phenyl- ethanol | 0.0008 |
| 19 | 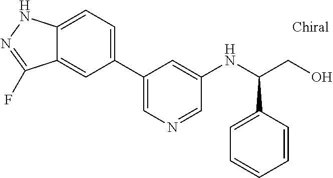 | (R)-2-[5-(3-Fluoro-1H- indazol-5-yl-pyridin-3- ylamino]-2-phenyl- ethanol | 0.0018 |
| 20 | 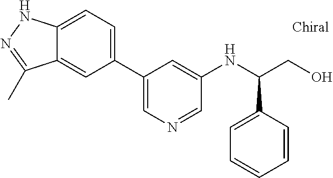 | (R)-2-[5-(3-Methyl-1H- indazol-5-yl)-pyridin-3- ylamino]-2-phenyl- ethanol | 0.0005 |
| 21 | 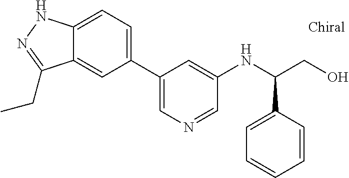 | (R)-2-[5-(3-Ethyl-1H- indazol-5-yl)-pyridin-3- ylamino]-2-phenyl- ethanol | 0.0015 |
| 22 | 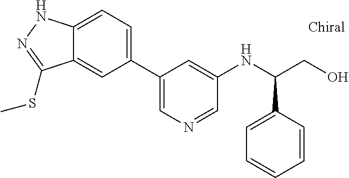 | (R)-2-[5-(3- Methylsulfanyl-1H- indazol-5-yl)-pyridin-3- ylamino]-2-phenyl- ethanol | 0.0005 |
| 23 | 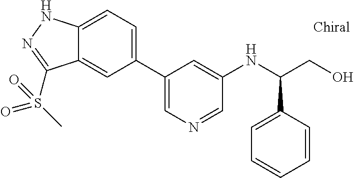 | (R)-2-[5-(3- Methanesulfonyl-1H- indazol-5-yl)-pyridin-3- ylamino]-2-phenyl- ethanol | 0.0032 |
| 24 | 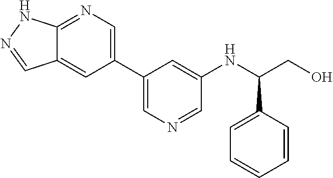 [SMILES: OC[C@H](Nc1cncc(-c2cnc3[nH]ncc3c2)c1)c1ccccc1] | (R)-2-Phenyl-2-[5-(1H- pyrazolo[3,4-b]pyridin-5- yl)-pyridin-3-ylamino]- ethanol | 0.0011 |
| 25 | 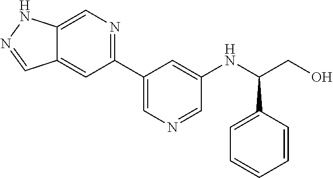 [SMILES: OC[C@H](Nc1cncc(-c2cc3cn[nH]c3cn2)c1)c1ccccc1] | (R)-2-Phenyl-2-[5-(1H- pyrazolo[3,4-c]pyridin-5- yl)-pyridin-3-ylamino]- ethanol | 0.0007 |
| 26 | 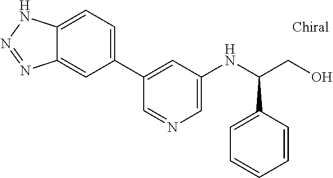 | (R)-2-[5-(1H- Benzotriazol-5-yl)- pyridin-3-ylamino]-2- phenyl-ethanol | 0.0094 |
| 27 | 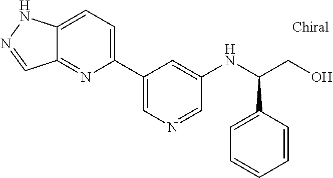 | (R)-2-Phenyl-2-[5-(1H- pyrazolo[4,3-b]pyridin-5- yl)-pyridin-3-ylamino]- ethanol | 0.0097 |
| 28 | 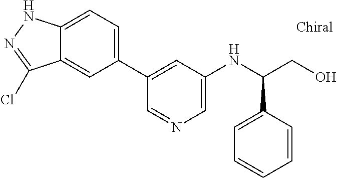 | (R)-2-[5-(3-Chloro-1H- indazol-5-yl)-pyridin-3- ylamino]-2-phenyl- ethanol | 0.001 |
| 29 | 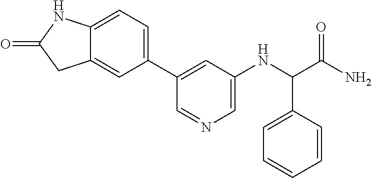 [SMILES: NC(=O)C(Nc1cncc(-c2ccc3c(c2)CC(=O)N3)c1)c1ccccc1] | 2-[5-(2-Oxo-2,3-dihydro- 1H-indol-5-yl)-pyridin-3- ylamino]-2-phenyl- acetamide | 0.0672 |
| 30 | 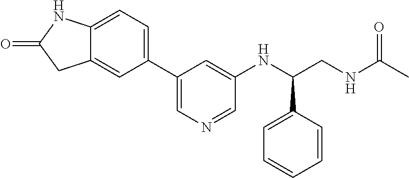 [SMILES: CC(=O)NC[C@H](Nc1cncc(-c2ccc3c(c2)CC(=O)N3)c1)c1ccccc1] | N-{(R)-2-[5-(2-Oxo-2,3- dihydro-1H-indol-5-yl)- pyridin-3-ylamino]-2- phenyl-ethyl}-acetamide | 0.0227 |
| 31 | 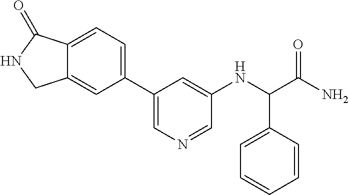 [SMILES: NC(=O)C(Nc1cncc(-c2ccc3c(c2)CNC3=O)c1)c1ccccc1] | 2-[5-(l-Oxo-2,3-dihydro- 1H-isoindol-5-yl)- pyridin-3-ylamino]-2- phenyl-acetamide | 0.294 |
| 32 | 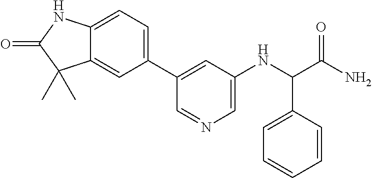 [SMILES: CC1(C)C(=O)Nc2ccc(-c3cncc(NC(C(N)=O)c4ccccc4)c3)cc21] | 2-[5-(3,3-Dimethyl-2- oxo-2,3-dihydro-1H- indol-5-yl)-pyridin-3- ylamino]-2-phenyl- acetamide | 0.8131 |
| 33 | 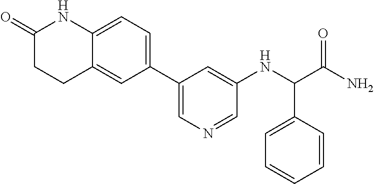 [SMILES: NC(=O)C(Nc1cncc(-c2ccc3c(c2)CCC(=O)N3)c1)c1ccccc1] | 2-[5-(2-Oxo-1,2,3,4- tetrahydro-quinolin-6-yl)- pyridin-3-ylamino]-2- phenyl-acetamide | 1.8928 |
| 34 | 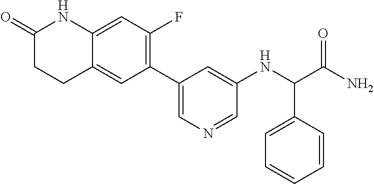 [SMILES: NC(=O)C(Nc1cncc(-c2cc3c(cc2F)NC(=O)CC3)c1)c1ccccc1] | 2-[5-(7-Fluoro-2-oxo- 1,2,3,4-tetrahydro- quinolin-6-yl)-pyridin-3- ylamino]-2-phenyl- acetamide | 1.3981 |
| 35 | 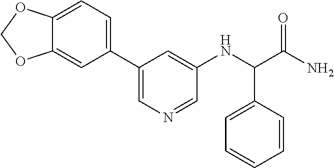 [SMILES: NC(=O)C(Nc1cncc(-c2ccc3c(c2)OCO3)c1)c1ccccc1] | 2-(5-Benzo[1,3]dioxol-5- yl-pyridin-3-ylamino)-2- phenyl-acetamide | 0.5591 |
| 36 | 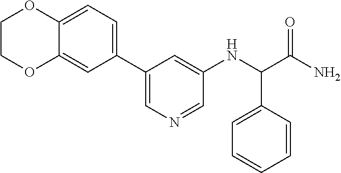 [SMILES: NC(=O)C(Nc1cncc(-c2ccc3c(c2)OCCO3)c1)c1ccccc1] | 2-[5-(2,3-Dihydro- benzo[1,4]dioxin-6-yl)- pyridin-3-ylamino]-2- phenyl-acetamide | 2.5146 |
| 37 | 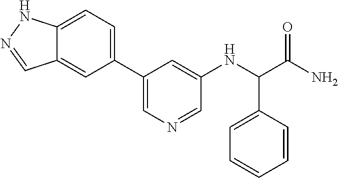 [SMILES: NC(=O)C(Nc1cncc(-c2ccc3[nH]ncc3c2)c1)c1ccccc1] | 2-[5-(1H-Indazol-5-yl)- pyridin-3-ylamino]-2- phenyl-acetamide | 0.0034 |
| 38 | 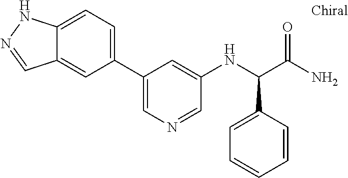 | (R)-2-[5-(1H-Indazol-5- yl)-pyridin-3-ylamino]-2- phenyl-acetamide | 0.0009 |
| 39 | 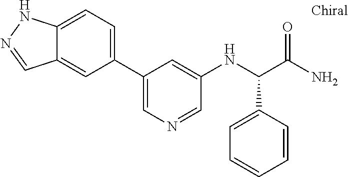 | (S)-2-[5-(1H-indazol-5- yl)-pyridin-3-ylamino]-2- phenyl-acetamide | 0.0088 |
| 40 | 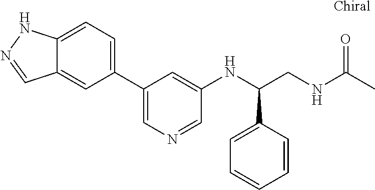 | N-{(R)-2-[5-(1H-Indazol- 5-yl)-pyridin-3-ylamino]- 2-phenyl-ethyl}-acetamide | 0.0018 |
| 41 | 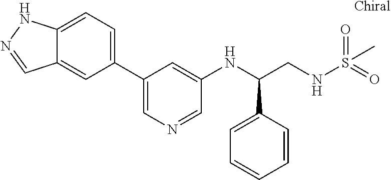 | N-{(R)-2-[5-(1H-Indazol- 5-yl)-pyridin-3-ylamino]- 2-phenyl-ethyl}- methanesulfonamide | 0.0003 |
| 42 | 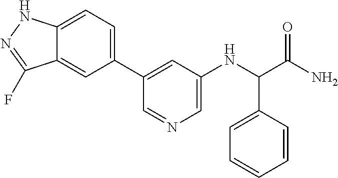 [SMILES: NC(=O)C(Nc1cncc(-c2ccc3[nH]nc(F)c3c2)c1)c1ccccc1] | 2-[5-(3-Fluoro-1H- indazol-5-yl)-pyridin-3- ylamino]-2-phenyl- acetamide | 0.0035 |
| 43 | 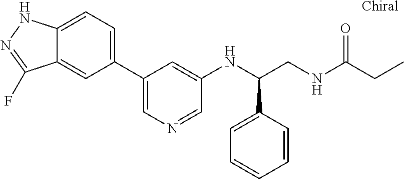 | N-{(R)-2-[5-(3-Fluoro- 1H-indazol-5-yl)-pyridin- 3-ylamino]-2-phenyl- ethyl}-propionamide | 0.0023 |
| 44 | 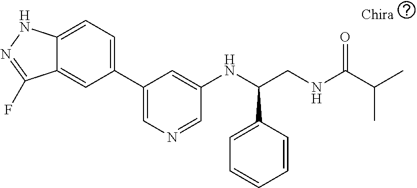 | N-{(R)-2-[5-(3-Fluoro- 1H-indazol-5-yl)-pyridin- 3-ylamino]-2-phenyl- ethyl}-isobutyramide | 0.0031 |
| 45 | 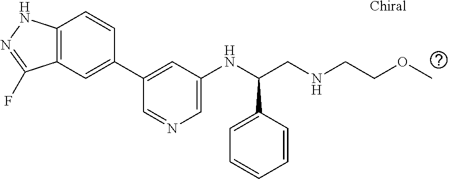 [SMILES: "Chiral" ; COCCNC[C@H](Nc1cncc(-c2ccc3[nH]nc(F)c3c2)c1)c1ccccc1] | (R)-N¹-[5-(3-Fluoro-1H- indazol-5-yl)-pyridin-3- yl]-N²-(2-methoxy-ethyl)- 1-phenyl-ethane-1,2- diamine | 0.0295 |
| 46 | 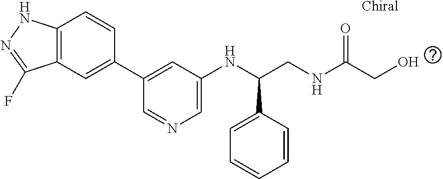 | N-{(R)-2-[5-(3-Fluoro- 1H-indazol-5-yl)-pyridin- 3-ylamino]-2-phenyl- ethyl}-2-hydroxy- acetamide | 0.0024 |
| 47 | 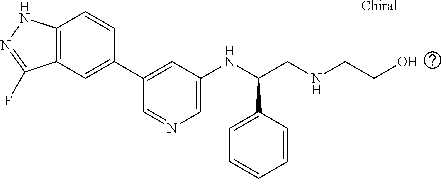 [SMILES: "Chiral" ; OCCNC[C@H](Nc1cncc(-c2ccc3[nH]nc(F)c3c2)c1)c1ccccc1] | 2-{(R)-2-[5-(3-Fluoro- 1H-indazol-5-yl)-pyridin- 3-ylamino]-2-phenyl- ethylamino}-ethanol | 0.015 |
| 48 | 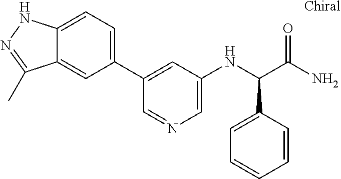 | (R)-2-[5-(3-Methyl-1H- indazol-5-yl)-pyridin-3- ylamino]-2-phenyl- acetamide | 0.0016 |
| 49 | 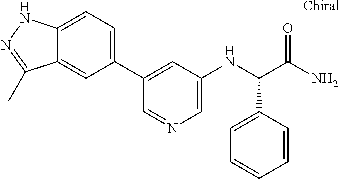 | (S)-2-[5-(3-Methyl-1H- indazol-5-yl)-pyridin-3- ylamino]-2-phenyl- acetamide | 0.0042 |
| 50 | 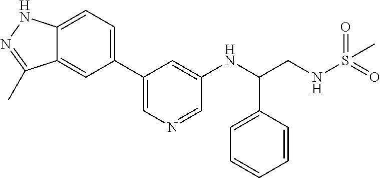 [SMILES: Cc1n[nH]c2ccc(-c3cncc(NC(CNS(C)(=O)=O)c4ccccc4)c3)cc12] | N-{2-[5-(3-Methyl-1H- indazol-5-yl)-pyridin-3- ylamino]-2-phenyl-ethyl}- methanesulfonamide | 0.0028 |
| 51 | 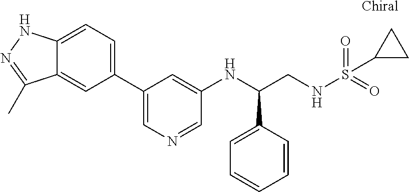 | Cyclopropanesulfonic acid {(R)-2-[5-(3-methyl- 1H-indazol-5-yl)-pyridin- 3-ylamino]-2-phenyl- ethyl}-amide | 0.0021 |
| 52 | 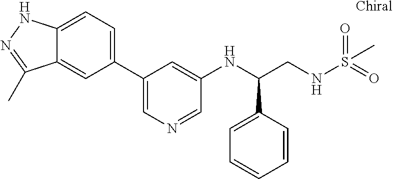 | N-{(R)-2-[5-(3-Methyl- 1H-indazol-5-yl)-pyridin- 3-ylamino]-2-phenyl- ethyl}- methanesulfonamide | 0.0008 |
| 53 | 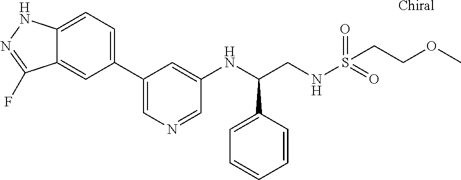 [SMILES: "Chiral" ; COCCS(=O)(=O)NC[C@H](Nc1cncc(-c2ccc3[nH]nc(F)c3c2)c1)c1ccccc1] | 2-Methoxy- ethanesulfonic acid {(R)- 2-[5-(3-methyl-1H- indazol-5-yl)-pyridin-3- ylamino]-2-phenyl- ethyl}-amide | 0.0017 |
| 54 | 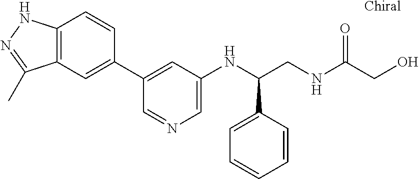 | 2-Hydroxy-N-{(R)-2-[5- (3-methyl-1H-indazol-5- yl)-pyridin-3-ylamino]-2- phenyl-ethyl}-acetamide | 0.0012 |
| 55 | 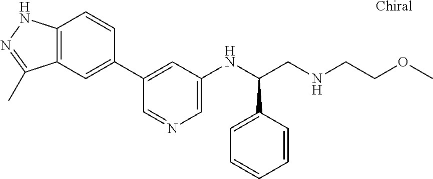 [SMILES: "Chiral" ; COCCNC[C@H](Nc1cncc(-c2ccc3[nH]nc(C)c3c2)c1)c1ccccc1] | (R)-N²-(2-Methoxy- ethyl)-N¹-[5-(3-methyl- 1H-indazol-5-yl)-pyridin- 3-yl]-1-phenyl-ethane- 1,2-diamine | 0.0095 |
| 56 | 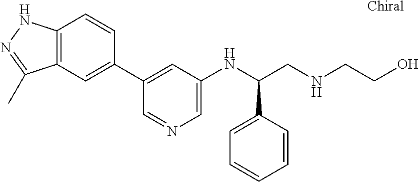 [SMILES: "Chiral" ; Cc1n[nH]c2ccc(-c3cncc(N[C@@H](CNCCO)c4ccccc4)c3)cc12] | 2-{(R)-2-[5-(3-Methyl- 1H-indazol-5-yl)-pyridin- 3-ylamino]-2-phenyl- ethylamino}-ethanol | 0.005 |
| 57 | 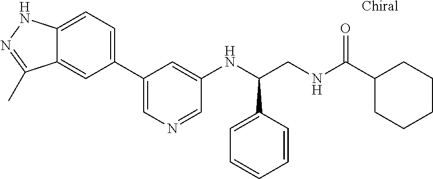 | Cyclohexanecarboxylic acid {(R)-2-[5-(3-methyl- 1H-indazol-5-yl)-pyridin- 3-ylamino]-2-phenyl- ethyl}-amide | 0.0053 |
| 58 | 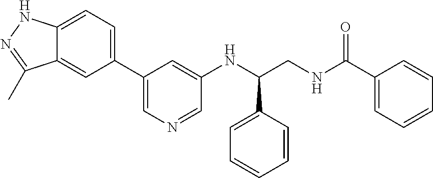 [SMILES: Cc1n[nH]c2ccc(-c3cncc(N[C@@H](CNC(=O)c4ccccc4)c4ccccc4)c3)cc12] | N-{(R)-2-[5-(3-Methyl- 1H-indazol-5-yl)-pyridin- 3-ylamino]-2-phenyl- ethyl}-benzamide | 0.006 |
| 59 | 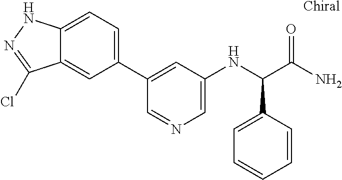 | (R)-2-[5-(3-Chloro-1H- indazol-5-yl)-pyridin-3- ylamino]-2-phenyl- acetamide | 0.0023 |
| 60 | 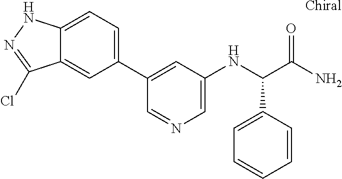 | (S)-2-[5-(3-Chloro-1H- indazol-5-yl)-pyridin-3- ylamino]-2-phenyl- acetamide | 0.0092 |

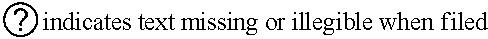

TABLE 2
NMR and MS data of particular compounds

| Ex- | | MS obsd. |
|---|---|---|
| ample | | (ESI⁺) |
| No. | ¹H NMR data | [(M + H)⁺] |
| 1 | ¹H NMR (METHANOL-d₄): δ 7.99-7.81 (m, 2 H), | 346.1 |
| | 7.51-7.43 (m, 2 H), 7.41-7.23 (m, 5 H), 7.10 | |
| | (m, 1 H), 6.95 (m, 1 H), 4.61-4.50 (m, 1 H), | |
| | 3.90-3.82 (m, 1 H), 3.80-3.69 (m, 1 H), 3.61-3.54 | |
| | (m, 1 H) | |
| 2 | ¹H NMR (METHANOL-d₄): δ 7.95 (d, 1 H), 7.87 | 374.1 |
| | (d, 1 H), 7.46 (m, 2 H), 7.38 (m, 2 H), 7.34-7.23 | |
| | (m, 3 H), 7.08 (t, 1 H), 6.98 (d, 1 H), 4.55 (dd, 1 H), | |
| | 4.01-3.83 (m, 1 H), 3.81-3.67 (m, 1 H), 1.38 (d, 6 H) | |
| 3 | ¹H NMR (METHANOL-d₄): δ 7.96 (br. s., 1H), 7.80 | 380.1 |
| | (br. s., 1H), 7.44-7.56 (m, 2H), 7.40 (s, 1H), 7.34 | |
| | (m, 1H), 7.29 (d, 2H), 7.05 (s, 1H), 6.97 (s, 1H), | |
| | 4.99-5.10 (m, 1H), 3.84-3.98 (m, 1H), 3.67-3.80 | |
| | (m, 1H) | |
| 4 | ¹H NMR (METHANOL-d₄): 7.88-8.04 (m, 1H), | 380.1 |
| | 7.80 (br. s., 1H), 7.43-7.57 (m, 2H), 7.40 (s, 1H), | |
| | 7.31-7.37 (m, 1H), 7.21-7.31 (m, 2H), 7.05 (s, 1H), | |
| | 6.97 (s, 1H), 4.99-5.10 (m, 1H), 3.91 (dd, 1H), 3.74 | |
| | (dd, 1H) | |
| 5 | ¹H NMR (DMSO-d₆): δ 10.70 (d, 2H), 7.83-7.96 | 347.2 |
| | (m, 2H), 7.42 (d, 2H), 7.32 (t, 2H), 7.19-7.26 (m, 1H), | |
| | 7.07 (d, 1H), 6.95-7.02 (m, 3H), 6.38 (d, 1H), 5.00 | |
| | (br. s., 1H), 4.51 (d, 1H), 3.65 (d, 2H) | |
| 6 | ¹H NMR (METHANOL-d₄): δ 7.89-7.98 (m, 1H), | 372.1 |
| | 7.80-7.88 (m, 1H), 7.41-7.51 (m, 2H), 7.37 (t, 2H), | |
| | 7.23-7.32 (m, 2H), 6.98-7.10 (m, 2H), 6.94 (d, 1H), | |
| | 4.47-4.62 (m, 1H), 3.81-3.89 (m, 1H), 3.72- | |
| | 3.80 (m, 1H), 1.67 (s, 4H) | |
| 7 | ¹H NMR (METHANOL-d₄): δ 8.15 (s, 1H), 7.93 | 347.2 |
| | (d, 2H), 7.73 (d, 1H), 7.42-7.47 (m, 2H), 7.36 | |
| | (t, 2H), 7.24-7.31 (m, 1H), 7.10 (s, 1H), 4.57 | |
| | (dd, 4.8 Hz, 1H), 3.82-3.89 (m, 1H), 3.74-3.80 | |
| | (m, 1H), 3.37 (s, 2H) | |
| 8 | ¹H NMR (METHANOL-d₄): δ 7.86-7.99 (m, 2H), | 382.2 |
| | 7.58-7.66 (m, 2H), 7.41-7.49 (m, 2H), 7.36 (t, 2H), | |
| | 7.25-7.30 (m, 1H), 7.10 (s, 1H), 7.04 (d, 1H), | |
| | 4.57 (dd, 1H), 3.83-3.89 (m, 1H), 3.74-3.81 (m, 1H) | |
| 9 | ¹H NMR (METHANOL-d₄): δ 8.26 (s, 1H), 7.89 | 347.2 |
| | (d, 1H), 7.54 (d, 1H), 7.44-7.47 (m, 3H), 7.35 (t, 2H), | |
| | 7.26 (d, 2H), 4.58 (dd, 1H), 3.83-3.88 (m, 1H), 3.74- | |
| | 3.80 (m, 1H) | |
| 10 | ¹H NMR (METHANOL-d₄): δ 7.86 (s, 2H), 7.39- | 364.2 |
| | 7.48 (m, 2H), 7.35 (t, 2H), 7.16-7.30 (m, 2H), 7.08 | |
| | (br. s., 1H), 6.74 (d, 1H), 4.52 (dd, 1H), 3.80-3.89 | |
| | (m, 1H), 3.71-3.80 (m, 1H) | |
| 11 | ¹H NMR (METHANOL-d₄): δ 7.93 (d, 1H), 7.87 | 364.2 |
| | (d, 1H), 7.41-7.49 (m, 2H), 7.36 (t, 2H), 7.28 (d, | |
| | 1H), 7.22 (d, 1H), 7.14 (dd, 1.0 Hz, 1H), 7.08 (t, | |
| | 1H), 4.56 (d, 1H), 3.81-3.91 (m, 1H), 3.71-3.81 | |
| | (m, 1H) | |
| 12 | ¹H NMR (METHANOL-d₄): δ 7.90(br. s., 2H) 7.41- | 348.3 |
| | 7.49 (m, 2H), 7.36 (t, 2H), 7.23-7.33 (m, 3H), 7.08- | |
| | 7.16 (m, 2H), 4.56 (dd, 1H), 3.82-3.91 (m, 1H), 3.70- | |
| | 3.82 (m, 1H) | |
| 13 | ¹H NMR (METHANOL-d₄): δ 7.95 (br. s., 1H), 7.84 | 364.2 |
| | (br. s., 1H), 7.53 (s, 1H), 7.43-7.47 (m, 2H), 7.36 (t, | |
| | 2H), 7.25-7.32 (m, 2H), 7.19 (d, 1H), 7.12 (s, 1H), | |
| | 4.56 (dd, 1H), 3.83-3.88 (m, 1H), 3.74-3.80 (m, 1H) | |
| 14 | ¹H NMR (DMSO-d₆) : δ 7.94 (d, 1 H), 7.90 (d, 1 H), | 335.2 |
| | 7.43 (d, 2 H), 7.32 (t, 2 H), 7.25-7.19 (m, 1 H), 7.07 | |
| | (s, 1 H), 7.02-6.93 (m, 3 H), 6.37 (d, 1 H), 6.05 (s, | |
| | 2 H), 5.01 (t, 1 H), 4.58-4.50 (m, 1 H), 3.73-3.58 (m, | |
| | 2 H) | |
| 15 | ¹H NMR (DMSO-d₆) : δ 7.93 (d, 1 H), 7.89 (d, 1 H), | 349.2 |
| | 7.43 (d, 2 H), 7.32 (t, 2 H), 7.26-7.19 (m, 1 H), 7.00- | |
| | 6.93 (m, 3 H), 6.93-6.87 (m, 1 H), 6.36 (d, 1 H), 5.00 | |
| | (t, 1 H), 4.58-4.47 (m, 1 H), 4.26 (s, 4 H), 3.72-3.56 | |
| | (m, 2 H) | |
| 16 | ¹H NMR (METHANOL-d₄) : δ 7.95 (d, 1 H), 7.87 | 331.1 |
| | (d, 1 H), 7.46 (d, 2 H), 7.38 (t, 2 H), 7.34-7.23 (m, 3 H), | |
| | 7.08 (t, 1 H), 6.98 (d, 1 H), 4.55 (dd, 1 H), 4.01-3.83 | |
| | (m, 1 H), 3.81-3.67 (m, 1 H), 1.38 (d, 6 H) | |
| 17 | ¹H NMR (METHANOL-d₄) : δ 8.04-7.93 (m, 2 H), | 330.1 |
| | 7.51-7.44 (m, 2 H), 7.41-7.36 (m, 3 H), 7.34-7.29 | |
| | (m, 1 H), 7.25-7.18 (m, 2 H), 7.15 (t, 1 H), 6.98 (d, | |
| | 1 H), 6.08 (d, 1 H), 4.53 (dd, 1 H), 3.90-3.83 (m, 1 H), | |
| | 3.80-3.72 (m, 1 H) | |
| 18 | ¹H NMR (METHANOL-d₄): δ 8.03 (d, 1H), 7.82- | 346.1 |
| | 7.88 (m, 2H), 7.41-7.50 (m, 3H), 7.33-7.40 (m, 3H), | |
| | 7.25-7.31 (m, 1H), 7.20 (s, 1H), 4.58 (dd, 1H), 3.82- | |
| | 3.90 (m, 1H), 3.74-3.82 (m, 1H) | |
| 19 | ¹H NMR (METHANOL-d₄): δ 8.01 (br. s., 1H), | 349.2 |
| | 7.89 (br. s., 1H), 7.71 (s, 1H), 7.45-7.57 (m, 4H), | |
| | 7.37 (t, 2H), 7.24-7.31 (m, 1H), 7.17 (br. s., 1H), | |
| | 4.58 (dd, 1H), 3.83-3.91 (m, 1H), 3.74-3.82 (m, 1H) | |
| 20 | ¹H NMR (METHANOL-d₄): δ 8.03 (s, 1H), 7.88 | 345.1 |
| | (d, 1H), 7.73 (s, 1H), 7.44-7.52 (m, 4H), 7.38 (t, 2H), | |
| | 7.27-7.32 (m, 1H), 7.18 (t, 1H), 4.58 (dd, 1H), | |
| | 3.84-3.91 (m, 1H), 3.75-3.82 (m, 1H), 2.58 (s, 3H) | |
| 21 | ¹H NMR (METHANOL-d₄): δ 8.03 (br. s., 1 H), | 359.1 |
| | 7.89 (br. s., 1 H), 7.72 (s, 1 H), 7.56-7.45 (m, 4 H), | |
| | 7.38 (t, 2 H), 7.32-7.25 (m, 1 H), 7.16 (s, 1 H), 4.57 | |
| | (dd, 1 H), 3.97-3.84 (m, 1 H), 3.81-3.74 (m, 1 H), | |
| | 3.02 (q, 2 H), 1.41 (t, 3 H) | |
| 22 | ¹HNMR(METHANOL-d₄): δ 8.01(s, 1H), 7.90(s, | 377.1 |
| | 1H), 7.66(s, 1H), 7.10-7.55(m, 8H), 4.56 (dd, 1H), | |
| | 3.87 (dd, 1H), 3.78 (dd, 1H), 2.60 (s, 3H) | |
| 23 | ¹HNMR(METHANOL-d₄): δ 8.12(s, 1H), 8.04(s, | 409.1 |
| | 1H), 7.92(s, 1H), 7.20-7.80(m, 8H), 4.59 (dd, 1H), | |
| | 3.87 (dd, 1H), 3.78 (dd, 1H), 3.77 (s, 3H) | |
| 24 | ¹H NMR (METHANOL-d₄): δ 8.61 (d, 1H), 8.32 (d, | 332.3 |
| | 1H), 8.17 (s, 1H), 8.03 (d, 1H), 7.94 (d, 1H), 7.47 (d, | |
| | 2H), 7.37 (t, 2H), 7.28 (d, 1H), 7.20 (t, 1H), 4.60 (m, | |
| | 1H), 3.84-3.92 (m, 1H), 3.72-3.83 (m, 1H) | |
| 25 | ¹H NMR (METHANOL-d₄): δ 9.06 (s, 1H), 8.36 (d, | 332.3 |
| | 1H), 8.23 (s, 1H), 8.09 (d, 1H), 7.91 (d, 1H), 7.58 (s, | |
| | 1H), 7.48 (d, 2H), 7.36 (t, 2H), 7.27 (d, 1H), 4.62 (m, | |
| | 1H), 3.84-3.93 (m, 1H), 3.72-3.82 (m, 1H) | |
| 26 | ¹H NMR (METHANOL-d₄): δ (d, 1 H), 7.98-7.91 | 332.3 |
| | (m, 3 H), 7.60 (d, 1 H), 7.48 (m, 2 H), 7.38 (t, 2 H), | |
| | 7.33-7.26 (m, 1 H), 7.23 (m, 1 H), 4.59 (dd, 1 H), | |
| | 3.93-3.84 (m, 1 H), 3.82-3.71 (m, 1 H) | |
| 27 | ¹H NMR (METHANOL-d₄): δ 8.37 (s, 1H), 8.27 (s, | 332.3 |
| | 1H), 8.09 (d, 1H), 7.96 (br. s., 1H), 7.76 (d, 1H), | |
| | 7.59 (d, 1H), 7.48 (d, 2H), 7.36 (t, 2H), 7.22-7.30 | |
| | (m, 1H), 4.63 (m, 1H), 3.83-3.92 (m, 1H), 3.73- | |
| | 3.83 (m, 1H) | |
| 28 | ¹H NMR (METHANOL-d₄): δ 8.03 (d, 1 H), 7.91 | 365.2 |
| | (d, 1 H), 7.67 (s, 1 H), 7.59 (s, 2 H), 7.48 (d, 2 H), | |
| | 7.39 (t, 2 H), 7.31-7.26 (m, 1 H), 7.19 (t, 1 H), 4.58 | |
| | (dd, 1 H), 3.95-3.84 (m, 1 H), 3.82-3.73 (m, 1 H) | |
| 29 | ¹H NMR (METHANOL-d₄): δ 8.05 (d, 1 H), 7.95 | 359 |
| | (d, 1 H), 7.59 (m, 2 H), 7.48 (d, 1 H), 7.45-7.30 | |
| | (m, 5 H), 7.24 (t, 1 H), 6.99 (d, 1 H), 5.06 (s, 1 H) | |
| 30 | ¹H NMR (METHANOL-d₄): δ 7.94 (d, 1 H), 7.82 | 387.2 |
| | (d, J = 2.5 Hz, 1 H), 7.49-7.42 (m, 2 H), 7.41-7.23 | |
| | (m, 5 H), 7.10 (s, 1 H), 6.96 (d, 1 H), 4.70-4.60 | |
| | (m, 1 H), 3.65-3.57 (m, 1 H), 3.56-3.48 (m, 1 H), | |
| | 1.96 (s, 3 H) | |
| 31 | ¹H NMR (DMSO-d₆): δ 8.59 (s, 1 H), 8.10 (d, 2 H), | 359.2 |
| | 7.76 (d, 3 H), 7.66 (d, 1 H), 7.57 (d, 2 H), 7.41-7.34 | |
| | (m, 2 H), 7.33-7.22 (m, 3 H), 6.62 (d, 1 H), 5.13 | |
| | (d, 1 H), 4.44 (s, 2 H) | |
| 32 | ¹H NMR (METHANOL-d₄): δ 8.07 (d, 1 H), 7.96 | 387 |
| | (d, 1 H), 7.60 (d, 2 H), 7.47-7.33 (m, 5 H), 7.24(d, | |
| | 1 H), 7.02 (d, 1 H), 5.07 (s, 1 H), 1.41 (d, 6 H) | |
| 33 | ¹H NMR (DMSO-d₆): δ 10.18 (s, 1 H), 8.05 (s, 1 H), | 373.2 |
| | 7.98 (d, 1 H), 7.73 (s, 1 H), 7.56 (d, 2 H), 7.42-7.23 | |
| | (m, 6 H), 7.18 (s, 1 H), 6.93 (d, 1 H), 6.54 (d, 1 H), | |
| | 5.09 (d, 1 H), 2.93 (t, 2 H), 2.49 (t, 2 H) | |
| 34 | ¹H NMR (DMSO-d₆): δ 10.25 (s, 1 H), 8.02 (d, 1 H), | 391.2 |
| | 7.90 (s, 1 H), 7.73 (br. s., 1 H), 7.55 (d, 2 H), 7.41- | |
| | 7.33 (m, 2 H), 7.33-7.22 (m, 3 H), 7.08 (br. s., 1 H), | |
| | 6.74 (d, 1 H), 6.55 (d, 1 H), 5.04 (d, 1 H), 2.90 (t, 2 H), | |
| | 2.50 (t, 2 H) | |
| 35 | ¹H NMR (DMSO-d₆): δ 8.01 (d, 2 H), 7.73 (br. s., | 348.2 |
| | 1 H), 7.56 (d, 2 H), 7.44-7.19 (m, 4 H), 7.14 (br. s., | |
| | 2 H), 7.08-6.95 (m, 2 H), 6.50 (d, 1 H), 6.07 (br. s., | |
| | 2 H), 5.10 (d, 1 H) | |
| 36 | ¹H NMR (DMSO-d₆): δ 8.01 (d, 1 H), 7.99 (d, 1 H), | 362.2 |
| | 7.72 (br. s., 1 H), 7.56 (d, 2 H), 7.40-7.33 (m, 2 H), | |
| | 7.33-7.27 (m, 1 H), 7.25 (br. s., 1 H), 7.12 (s, 1 H), | |
| | 7.07-7.01 (m, 2 H), 6.94 (d, 1 H), 6.50 (d, 1 H), 5.09 | |
| | (d, 1 H), 4.28 (s, 4 H) | |
| 37 | ¹H NMR (METHANOL-d₄): δ 8.06-8.22 (m, 2H), | 344.2 |
| | 7.94-8.03 (m, 2H), 7.52-7.74 (m, 4H), 7.25-7.51 | |
| | (m, 4H), 5.09 (s, 1H) | |
| 38 | ¹H NMR (METHANOL-d₄): δ 8.12 (s, 2H), 7.97 | 344.2 |
| | (s, 2H), 7.54-7.69 (m, 4H), 7.28-7.47 (m, 4H), 5.08 | |
| | (s, 1H) | |
| 39 | ¹H NMR (METHANOL-d₄): δ 8.07-8.19 (m, 2H), | 344.2 |
| | 7.97 (s, 2H), 7.54-7.70 (m, 4H), 7.30-7.48 (m, 4H), | |
| | 5.08 (s, 1H) | |
| 40 | ¹H NMR (METHANOL-d₄): δ 8.11 (s, 1 H), 8.03 (d, | 372.2 |
| | 1 H), 7.90 (s, 1 H), 7.85 (d, 1 H), 7.65-7.58 (m, 1 H), | |
| | 7.57-7.51 (m, 1 H), 7.50-7.44 (m, 2 H), 7.39 (t, 2 H), | |
| | 7.33-7.26 (m, 1 H), 7.20 (t, 1 H), 4.68 (dd, 1 H), 3.68- | |
| | 3.58 (m, 1 H), 3.57-3.49 (m, 1 H), 1.97 (s, 3 H) | |
| 41 | ¹H NMR (DMSO-d₆): δ 13.15 (br. s., 1 H), 8.13 (s, 1 H), | 408.2 |
| | 8.08 (s, 1 H), 7.93 (s, 2 H), 7.65-7.59 (m, 1 H), 7.56- | |
| | 7.51 (m, 1 H), 7.49 (d, 2 H), 7.36 (t, 2 H), 7.30-7.21 | |
| | (m, 2 H), 7.15 (s, 1 H), 6.49 (d, 1 H), 4.73-4.63 (m, 1 H), | |
| | 2.86 (s, 3 H) | |
| 42 | ¹HNMR(METHANOL-d₄): δ 8.13(brs, 1H), 7.98(brs, | 362.2 |
| | 1H), 7.83(s, 1H), 7.50-7.70(m, 4H), 7.30-7.45(m, | |
| | 4H), 5.08(s, 1H) | |
| 43 | ¹H NMR (METHANOL-d₄): δ 8.01 (s, 1 H), 7.87 (d, | 404.3 |
| | 1 H), 7.74 (s, 1 H), 7.61-7.54 (m, 1 H), 7.53-7.43 (m, | |
| | 3 H), 7.38 (t, 2 H), 7.32-7.26 (m, 1 H), 7.17 (s, 1 H), | |
| | 4.69 (dd, 1 H), 3.69-3.59 (m, 1 H), 3.58-3.48 (m, 1 H), | |
| | 2.22 (q, 2 H), 1.12 (t, 3 H). | |
| 44 | ¹H NMR (METHANOL-d₄): δ 8.01 (s, 1 H), 7.87 (d, | 418.3 |
| | 1 H), 7.74 (s, 1 H), 7.61-7.44 (m, 4 H), 7.38 (t, 2 H), | |
| | 7.32-7.26 (m, 1 H), 7.17 (s, 1 H), 4.73-4.65 (m, 1 H), | |
| | 3.69-3.59 (m, 1 H), 3.58-3.49 (m, 1 H), 2.50-2.37 (m, | |
| | 1 H), 1.09 (t, 6 H). | |
| 45 | ¹HNMR(METHANOL-d₄): δ 8.09(s, 1H), 7.98(s, 1H), | 406.3 |
| | 7.74(s, 1H), 7.25-7.60(m, 8H), 5.05(t, 1H), 3.70- | |
| | 3.73(m, 2H), 3.40-3.45(m, 4H), 3.30-3.38(m, 3H) | |
| 46 | ¹HNMR(METHANOL-d₄): δ 8.02(s, 1H), 7.86(s, 1H), | 406.2 |
| | 7.73(s, 1H), 7.10-7.7.55(m, 8H), 4.70(dd, 1H), 4.01 | |
| | (s, 2H), 3.60-3.70 (m, 2H) | |
| 47 | ¹HNMR (METHANOL-d₄): δ 8.11(s, 1H), 7.99(s, 1H), | 392.2 |
| | 7.76(s, 1H), 7.30-7.60(m, 8H), 5.08 (dd, 1H), 3.89 | |
| | (t, 2H), 3.40-3.5255 (m, 2H), 3.29 (t, 2H) | |
| 48 | ¹HNMR(METHANOL-d₄): δ 8.12(s, 1H), 8.02(s, 1H), | 358.3 |
| | 7.86(s, 1H), 7.50-7.65(m, 4H), 7.30-7.45(m, 4H), 5.09 | |
| | (s, 1H), 2.61(s, 3H) | |
| 49 | ¹HNMR(METHANOL-d₄): δ 8.12(s, 1H), 8.02(s, 1H), | 358.2 |
| | 7.86(s, 1H), 7.50-7.65(m, 4H), 7.30-7.45(m, 4H), 5.09 | |
| | (s, 1H), 2.61(s, 3H) | |
| 50 | ¹H NMR (METHANOL-d₄): δ 8.10 (s, 1 H), 7.92 (d, | 422.2 |
| | 1 H), 7.83 (s, 1 H), 7.57-7.44 (m, 4 H), 7.42-7.19 (m, | |
| | 4 H), 7.14 (br. s., 1 H), 6.46 (d, 1 H), 4.67 (d, 1 H), | |
| | 3.41-3.25 (m, 4 H), 2.86 (s, 3 H), 2.52 (br. s., 3 H) | |
| 51 | ¹H NMR (METHANOL-d₄): δ 8.07 (s, 1 H), 7.89 (d, | 448.1 |
| | 1 H), 7.79 (s, 1 H), 7.55-7.48 (m, 4 H), 7.41 (t, 2 H), | |
| | 7.35-7.29 (m, 1 H), 7.25 (s, 1 H), 4.67 (t, 1 H), 3.52 | |
| | (d, 1 H), 2.56-2.47 (m, 1 H), 1.06 (d, 2 H), 0.98 (d, 2 H) | |
| 52 | ¹H NMR (DMSO-d₆): δ 12.70 (s, 1 H), 8.10 (s, 1 H), | 422.2 |
| | 7.92 (br. s., 1 H), 7.83 (s, 1 H), 7.57-7.44 (m, 4 H), | |
| | 7.42-7.21 (m, 4 H), 7.14 (br. s., 1 H), 6.46 (d, 1 H), | |
| | 4.66 (d, 1 H), 3.33 (m, 1 H), 3.09 (m, 1 H), 2.85 (s, 3 | |
| | H), 2.53 (s, 3 H) | |
| 53 | ¹H NMR (DMSO-d₆): δ 12.69 (br. s., 1 H), 8.10 (s, 1 H), | 466.2 |
| | 7.92 (d, 1 H), 7.83 (s, 1 H), 7.56-7.43 (m, 4 H), 7.37 (t, | |
| | 2 H), 7.31-7.22 (m, 2 H), 7.14 (br. s., 1 H), 6.42 (d, 1 H), | |
| | 4.65 (q, 1 H), 3.61 (t, 2 H), 3.29 (t, 2 H), 3.20 (s, 3 H), | |
| | 2.52 (br. s., 3 H) | |
| 54 | ¹H NMR(METHANOL-d₄): δ 8.03(s, 1H), 7.85(s, 1H), | 402.2 |
| | 7.73(s, 1H), 7.10-7.7.55(m, 8H), 4.70(dd, 1H), 4.01 | |
| | (s, 2H), 3.60-3.70(m, 2H), 2.57(s, 3H) | |
| 55 | ¹H NMR (METHANOL-d₄): δ 8.07(s, 1H), 7.92(s, 1H), | 402.3 |
| | 7.77(s, 1H), 7.20-7.55(m, 8H), 4.80 (t, 1H), 3.58 (dd, | |
| | 2H), 3.38 (s, 3H), 3.12 (d, 2H), 3.03 (t, 2H), 2.60 (s, 3H) | |
| 56 | ¹H NMR(METHANOL-d₄): δ 8.15(s, 1H), 7.98(s, 1H), | 388.2 |
| | 7.81(s, 1H), 7.30-7.60(m, 8H), 5.06 (dd, 1H), 3.88 (t, | |
| | 2H), 3.39-3.52 (m, 2H), 3.28 (t, 2H), 2.60 (s, 3H) | |
| 57 | ¹H NMR (METHANOL-d₄): δ 8.04 (br. s., 1 H), 7.85 | 454.4 |
| | (br. s., 1 H), 7.77 (s, 1 H), 7.53 (s, 2 H), 7.50-7.44 (m, | |
| | 2 H), 7.39 (t, 2 H), 7.34-7.27 (m, 1 H), 7.19 (br. s., 1 H), | |
| | 4.69 (t, 1 H), 3.69-3.59 (m, 1 H), 3.57-3.48 (m, 1 H), | |
| | 2.67-2.52 (m, 3 H), 2.26-2.12 (m, 1 H), 1.82-1.65 (m, | |
| | 4 H), 1.50-1.22 (m, 6 H) | |
| 58 | ¹H NMR (DMSO-d₆): δ 12.69 (br. s., 1 H), 8.62 (t, 1 H), | 448.4 |
| | 8.08 (s, 1 H), 7.92 (d, 1 H), 7.86-7.76 (m, 3 H), 7.55- | |
| | 7.42 (m, 7 H), 7.36 (t, 2 H), 7.27 (d, 1 H), 7.16 (br. s., | |
| | 1 H), 6.57 (d, 1 H), 4.81 (q, 1 H), 3.78-3.68 (m, 1 H), | |
| | 3.66-3.56 (m, 1 H), 2.52 (br. s., 3 H) | |
| 59 | ¹H NMR (METHANOL-d₄): δ 8.14 (d, 1 H), 8.01 (d, | 378.1 |
| | 1 H), 7.81 (s, 1 H), 7.73-7.67 (m, 1 H), 7.66-7.58 | |
| | (m, 3 H), 7.47-7.40 (m, 2 H), 7.40-7.27 (m, 2 H), | |
| | 5.10 (s, 1 H) | |
| 60 | ¹H NMR (METHANOL-d₄): δ 8.14 (s, 1 H), 8.01 | 378.1 |
| | (d, 1 H), 7.81 (s, 1 H), 7.72-7.67 (m, 1 H), 7.65- | |
| | 7.58 (m, 3 H), 7.49-7.40 (m, 2 H), 7.39-7.28 (m, | |
| | 2 H), 5.10 (s, 1 H) | |

TABLE 3
IC₅₀ on HCT116, SW-837 or AGS of particular compounds

| | IC₅₀ (μM) on | | |
|---|---|---|---|
| Example | HCT116 (colorectal | IC₅₀ (μM) on SW-837 | IC₅₀ (μM) on AGS |
| No. | cancer) | (colorectal cancer) | (gastric cancer) |
| 1 | 0.66 | 2.3 | 0.65 |
| 16 | 0.037 | 0.091 | 0.033 |

More particular compounds of formula I include the following:
5-[5-((R)-2-Hydroxy-1-phenyl-ethylamino)-pyridin-3-yl]-1,3-dihydro-indol-2-one;
5-[5-((R)-2-Hydroxy-1-phenyl-ethylamino)-pyridin-3-yl]-1,3-dihydro-benzoimidazol-2-one;
(R)-5′-(5-((2-Hydroxy-1-phenylethyl)amino)pyridin-3-yl)-spiro[cyclopropane-1,3′-indolin]-2′-one;
6-Fluoro-5-[5-((R)-2-hydroxy-1-phenyl-ethylamino)-pyridin-3-yl]-1,3-dihydro-indol-2-one;
6-[5-((R)-2-Hydroxy-1-phenyl-ethylamino)-pyridin-3-yl]-3H-benzooxazol-2-one;
6-[5-((R)-2-Hydroxy-1-phenyl-ethylamino)-pyridin-3-yl]-3H-benzothiazol-2-one;
(R)-2-[5-(1H-Indazol-5-yl)-pyridin-3-ylamino]-2-phenyl-ethanol;
(R)-2-[5-(3-Amino-1H-indazol-5-yl)-pyridin-3-ylamino]-2-phenyl-ethanol;
(R)-2-[5-(3-Fluoro-1H-indazol-5-yl-pyridin-3-ylamino]-2-phenyl-ethanol;
(R)-2-[5-(3-Methyl-1H-indazol-5-yl)-pyridin-3-ylamino]-2-phenyl-ethanol;
(R)-2-[5-(3-Ethyl-1H-indazol-5-yl)-pyridin-3-ylamino]-2-phenyl-ethanol;
(R)-2-[5-(3-Methylsulfanyl-1H-indazol-5-yl)-pyridin-3-ylamino]-2-phenyl-ethanol;
(R)-2-[5-(3-Methanesulfonyl-1H-indazol-5-yl)-pyridin-3-ylamino]-2-phenyl-ethanol;
(R)-2-Phenyl-2-[5-(1H-pyrazolo[3,4-b]pyridin-5-yl)-pyridin-3-ylamino]-ethanol;
(R)-2-Phenyl-2-[5-(1H-pyrazolo[3,4-c]pyridin-5-yl)-pyridin-3-ylamino]-ethanol;
(R)-2-Phenyl-2-[5-(1H-pyrazolo[4,3-b]pyridin-5-yl)-pyridin-3-ylamino]-ethanol;
(R)-2-[5-(3-Chloro-1H-indazol-5-yl)-pyridin-3-ylamino]-2-phenyl-ethanol;
2-[5-(2-Oxo-2,3-dihydro-1H-indol-5-yl)-pyridin-3-ylamino]-2-phenyl-acetamide;
2-[5-(7-Fluoro-2-oxo-1,2,3,4-tetrahydro-quinolin-6-yl)-pyridin-3-ylamino]-2-phenyl-acetamide;
2-[5-(1H-Indazol-5-yl)-pyridin-3-ylamino]-2-phenyl-acetamide;
(R)-2-[5-(1H-Indazol-5-yl)-pyridin-3-ylamino]-2-phenyl-acetamide;
N-{(R)-2-[5-(1H-Indazol-5-yl)-pyridin-3-ylamino]-2-phenyl-ethyl}-acetamide;
N-{(R)-2-[5-(1H-Indazol-5-yl)-pyridin-3-ylamino]-2-phenyl-ethyl}-methanesulfonamide;
2-[5-(3-Fluoro-1H-indazol-5-yl)-pyridin-3-ylamino]-2-phenyl-acetamide;
N-{(R)-2-[5-(3-Fluoro-1H-indazol-5-yl)-pyridin-3-ylamino]-2-phenyl-ethyl}-propionamide;
N-{(R)-2-[5-(3-Fluoro-1H-indazol-5-yl)-pyridin-3-ylamino]-2-phenyl-ethyl}-isobutyramide;
(R)-N^{l}-[5-(3-Fluoro-1H-indazol-5-yl)-pyridin-3-yl]-N²-(2-methoxy-ethyl)-1-phenyl-ethane-1,2-diamine;
N-{(R)-2-[5-(3-Fluoro-1H-indazol-5-yl)-pyridin-3-ylamino]-2-phenyl-ethyl}-2-hydroxy-acetamide;
2-{(R)-2-[5-(3-Fluoro-1H-indazol-5-yl)-pyridin-3-ylamino]-2-phenyl-ethylamino}-ethanol;
(R)-2-[5-(3-Methyl-1H-indazol-5-yl)-pyridin-3-ylamino]-2-phenyl-acetamide;
(S)-2-[5-(3-Methyl-1H-indazol-5-yl)-pyridin-3-ylamino]-2-phenyl-acetamide;
N-{2-[5-(3-Methyl-1H-indazol-5-yl)-pyridin-3-ylamino]-2-phenyl-ethyl}-methanesulfonamide;
Cyclopropanesulfonic acid {(R)-2-[5-(3-methyl-1H-indazol-5-yl)-pyridin-3-ylamino]-2-phenyl-ethyl}-amide;
N-{(R)-2-[5-(3-Methyl-1H-indazol-5-yl)-pyridin-3-ylamino]-2-phenyl-ethyl}-methanesulfonamide;
2-Methoxy-ethanesulfonic acid {(R)-2-[5-(3-methyl-1H-indazol-5-yl)-pyridin-3-ylamino]-2-phenyl-ethyl}-amide;
2-Hydroxy-N-{(R)-2-[5-(3-methyl-1H-indazol-5-yl)-pyridin-3-ylamino]-2-phenyl-ethyl}-acetamide;
(R)-N²-(2-Methoxy-ethyl)-N^{l}-[5-(3-methyl-1H-indazol-5-yl)-pyridin-3-yl]-1-phenyl-ethane-1,2-diamine;
2-{(R)-2-[5-(3-Methyl-1H-indazol-5-yl)-pyridin-3-ylamino]-2-phenyl-ethylamino}-ethanol;
Cyclohexanecarboxylic acid {(R)-2-[5-(3-methyl-1H-indazol-5-yl)-pyridin-3-ylamino]-2-phenyl-ethyl}-amide;
N-{(R)-2-[5-(3-Methyl-1H-indazol-5-yl)-pyridin-3-ylamino]-2-phenyl-ethyl}-benzamide;
(R)-2-[5-(3-Chloro-1H-indazol-5-yl)-pyridin-3-ylamino]-2-phenyl-acetamide; and
(S)-2-[5-(3-Chloro-1H-indazol-5-yl)-pyridin-3-ylamino]-2-phenyl-acetamide.

### Synthesis

The compounds of the present invention can be prepared by any conventional means. Suitable processes for synthesizing these compounds as well as their starting materials are provided in the schemes below and in the examples. All substituents, in particular, R¹, R² and R³ are as defined above unless otherwise indicated. Furthermore, and unless explicitly otherwise stated, all reactions, reaction conditions, abbreviations and symbols have the meanings well known to a person of ordinary skill in organic chemistry.

General synthetic route for intermediate (Scheme 1)

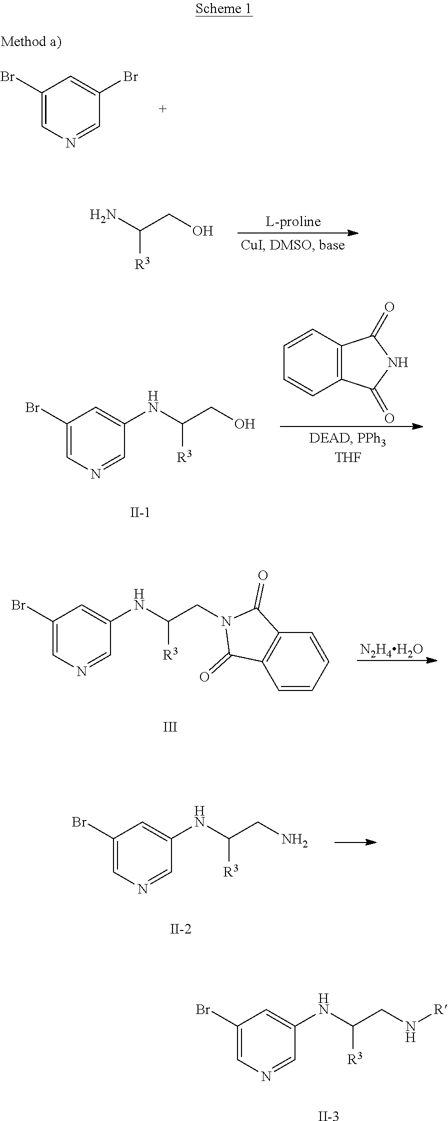

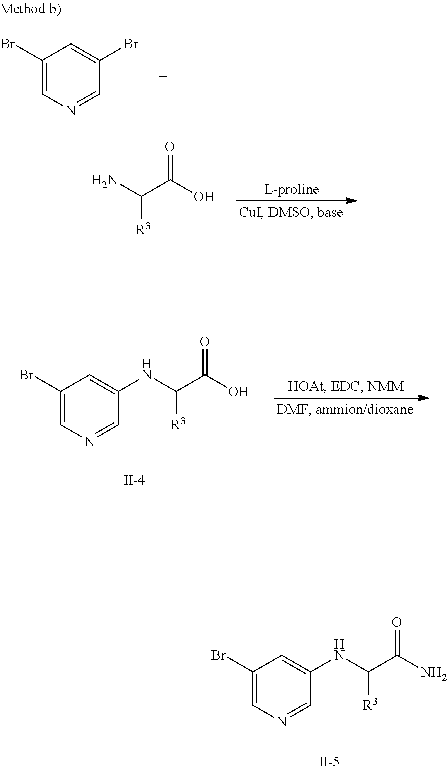

R′ is C₁₋₆alkylsulfonyl, C₁₋₆alkyl or C₁₋₆alkoxycarbonyl.

Intermediates II-1 to II-7 can be prepared according to Scheme 1.

By Method a), coupling between 3, 5-dibromo-pyridine and amino alcohol affords intermediate II-1. The reaction can be carried out in the presence of copper catalyst, and a ligand such as dimethylamino acetic acid or L-proline, and a suitable base such as K₂CO₃ or Cs₂CO₃, in a suitable solvent such as DMSO or 1,4-dioxane.

Intermediate III can be synthesized via Mitsunobu reaction between compound II-1 and isoindole-1,3-dione. The reaction can be carried out in the presence of DEAD and PPh₃ in THF. Ammonolyze of intermediate III affords compound II-2. Connection between compound II-2 and C₁₋₆alkylsulfonyl chloride, C₁₋₆alkyl or C₁₋₆alkyl acid affords intermediate II-3.

By Method b), coupling between 3,5-dibromo-pyridine and amino acetic acid affords intermediate II-4. The reaction can be carried out in the presence of copper catalysis, and a suitable ligand such as dimethylamino acetic acid or L-proline, and a suitable base such as K₂CO₃ or Cs₂CO₃, in a suitable solvent such as DMSO or 1,4-dioxane. Connection between compound II-4 and ammonia solution in the presence of HATU and DIPEA affords intermediate II-5.

General synthetic route for formula Ia (Scheme 2)

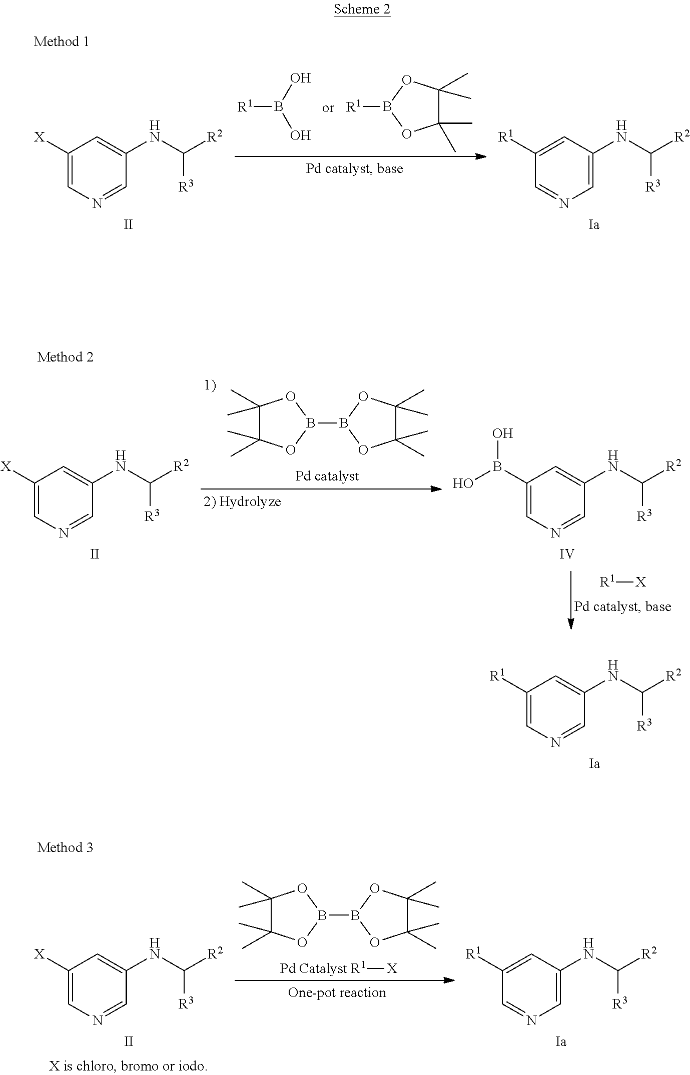

The compound of formula Ia can be prepared according to Scheme 2.

By Method 1, coupling between compound II and boronic acid or boronic ester affords Ia. The reaction can be carried out in the presence of Pd catalyst such as Pd(PPh₃)₄ or PdCl₂(PPh₃)₂, and a suitable base such as K₃PO₄, Na₂CO₃, K₂CO₃ or Cs₂CO₃, in a suitable solvent such as DME/H₂O, 1,4-dioxane/H₂O or DMF/H₂O.

By Method 2, boronic acid IV can be prepared by the reaction of intermediate II and bis(pinacolato)diboron in the presence of Pd catalyst and followed by hydrolyze reaction. Then coupling between intermediate IV and halide affords compound Ia.

By Method 3, compound Ia can be prepared by one-pot reaction. Compound II reacts with bis(pinacolato)diboron, and in the presence of Pd catalyst such as tris(dibenzylideneacetone) dipalladium and a ligand such as butyldi-1-adamantylphosphine, then halide R¹—X is added and the mixture is stirred at 100° C. for several hours under microwave to afford compound Ia.

General synthetic route for formulas Ib and Ic (Scheme 3)

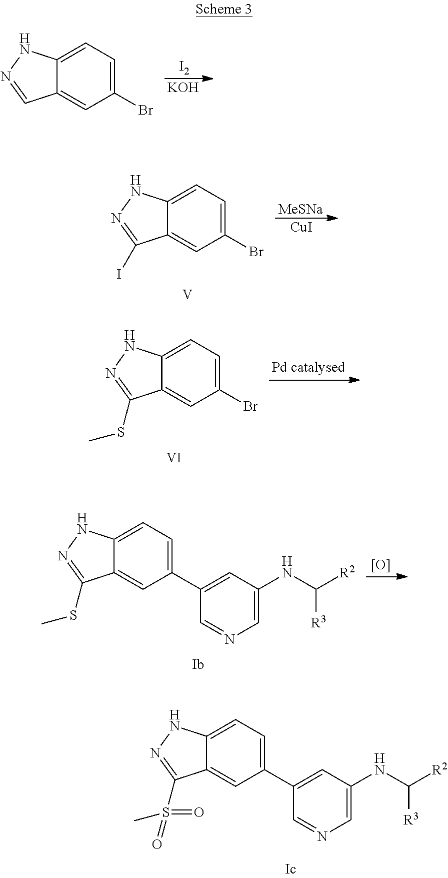

The compounds of formulas Ib and Ic can be prepared according to Scheme 3. Intermediate V can be synthesized via the introduction of iodine to the 3-position of indazole. Compound VI can be prepared by intermediate V and MeSNa solution in the presence of CuI One-pot reaction as described in Method 3 in Scheme 2 affords compound Ib. Oxidization of the compound Ib in the presence of oxone in DMF affords compound Ic.

General synthetic route for formula Id and Ie (Scheme 4)

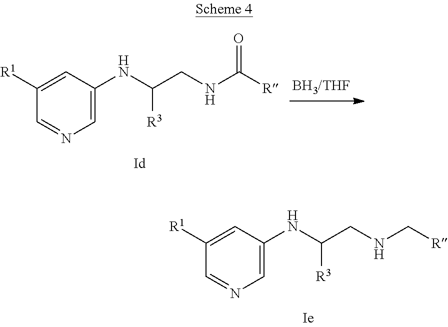

R″ is C₁₋₆ alkyl or C₁₋₆alkoxy-CH₂—.

The compound of formula Ie can be prepared according to Scheme 4. Reduction of amide Id in the presence of BH₃ in THF at 80° C. overnight affords Ie.

This invention also relates to a process for the preparation of a compound of formula I comprising the reaction of

(a) a compound of formula (A)

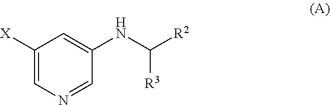

with

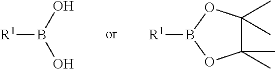

in the presence of a catalyst and a base;

(b) a compound of formula (B)

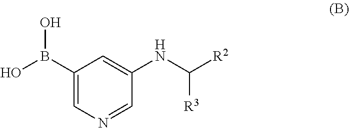

with R¹—X in the presence of a catalyst and a base;

(c) a compound of formula (A)

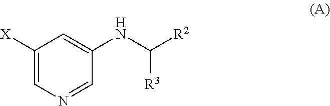

with bis(pinacolato)diboron and R¹—X in the presence of a catalyst and a ligand under microwave;

(d) a compound of formula (C)

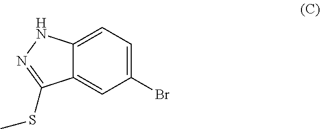

in the presence of a catalyst;

(e) a compound of formula (D)

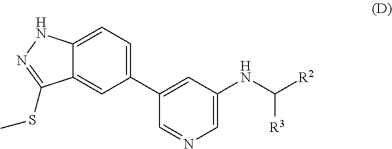

in the presence of oxone;

(f) a compound of formula (E)

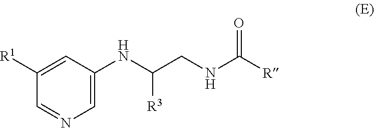

in the presence of BH₃;

wherein R¹, R² and R³are defined above unless otherwise indicated; X is chloro, bromo or iodo; R″ is C₁₋₆alkyl or C₁₋₆alkoxy-CH₂—.

In step (a), the catalyst can be for example Pd(PPh₃)₄, PdCl₂(PPh₃)₂, the base can be for example K₃PO₄, Na₂CO₃, K₂CO₃ or Cs₂CO₃;

In step (b), the catalyst can be for example Pd(PPh₃)₄, the base can be for example K₂CO₃;

In step (c), the catalyst can be for example tris(dibenzylideneacetone) dipalladium, the ligand can be for example butyldi-l-adamantylphosphine;

In step (d), the catalyst can be for example Pd(dppf)Cl₂.

A compound of formula I when manufactured according to the above process is also an object of the invention.

### Pharmaceutical Compositions and Administration

The invention also relates to a compound of formula I for use as therapeutically active substance.

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but particularly ranges anywhere from about 3 to about 8. In one example, a compound of formula I is formulated in an acetate buffer, at pH 5. In another embodiment, the compounds of formula I are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit CDK8 activity. For example, such amount may be below the amount that is toxic to normal cells, or the mammal as a whole.

In one example, the pharmaceutically effective amount of the compound of the invention administered parenterally per dose will be in the range of about 0.1 to 50 mg/kg of patient body weight per day, with the typical initial range of compound used being about 0.3 to about 15 mg/kg/day. In another embodiment, oral unit dosage forms, such as tablets and capsules, preferably contain from about 5 mg to about 500 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 5 mg to 500 mg of the compound of the invention compounded with about 90 mg to 30 mg anhydrous lactose, about 5 mg to 40 mg sodium croscarmellose, about 5 mg to 30 mg polyvinylpyrrolidone (PVP) K30, and about 1 mg to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 mg to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula I, or a stereoisomer or pharmaceutically acceptable salt thereof. In a further embodiment includes a pharmaceutical composition comprising a compound of Formula I, or a stereoisomer or pharmaceutically acceptable salt thereof, together with a pharmaceutically acceptable carrier or excipient.

Another embodiment includes a pharmaceutical composition comprising a compound of Formula I for use in the treatment of a hyperproliferative disease. Another embodiment includes a pharmaceutical composition comprising a compound of Formula I for use in the treatment of cancer.

### Indications and Methods of Treatment

The compounds of the invention inhibit the kinase activity of protein. Accordingly, the compounds of the invention are useful for inhibiting cell proliferation and inducing cell cycle arrest and apoptosis in particular cancer cells.

Compounds of the invention are useful for inhibiting cell proliferation, inducing cell cycle arrest and apoptosis in cells that overexpress CDK8 or Cyclin C.

Alternatively, compounds of the invention are useful for inhibiting cell proliferation, inducing cell cycle arrest and apoptosis in cells in which the apoptotic pathway is disrupted or proliferation pathway is overexpressed/or immortalized, for example by deregulation of CDK8 or Cyclin C.

The compounds of inventions are useful as inhibitors of CDK8 or Cyclin C.

An embodiment of this invention includes the use of a compound for the treatment of cancer, in particular bladder, head and neck, breast, stomach, ovary, colon, lung, brain, larynx, lymphatic system, liver, skin, hematopoetic system, genitourinary tract, gastrointestinal, ovarian, prostate, gastric, bone, small-cell lung, glioma, colorectal and pancreatic cancers. A further embodiment of this invention includes the use of a compound for the treatment of gastric cancer or colorectal cancer.

Another embodiment of this invention includes the use of a compound for the preparation of a medicament for the treatment of cancer, in particular bladder, head and neck, breast, stomach, ovary, colon, lung, brain, larynx, lymphatic system, liver, skin, hematopoetic system, genitourinary tract, gastrointestinal, ovarian, prostate, gastric, bone, small-cell lung, glioma, colorectal and pancreatic cancers.

A further embodiment of this invention includes the use of a compound for the preparation of a medicament for the treatment of gastric cancer or colorectal cancer.

Another embodiment of this invention relates to a compound of formula I for the treatment of cancer, in particular bladder, head and neck, breast, stomach, ovary, colon, lung, brain, larynx, lymphatic system, liver, skin, hematopoetic system, genitourinary tract, gastrointestinal, ovarian, prostate, gastric, bone, small-cell lung, glioma, colorectal and pancreatic cancers.

A further embodiment of this invention relates to a compound of formula I for the treatment of gastric cancer or colorectal cancer.

Another embodiment includes a method of treating or preventing cancer in a mammal in need of such treatment, wherein the method comprises administering to said mammal a therapeutically effective amount of a compound of Formula I, a stereoisomer, tautomer, prodrug or pharmaceutically acceptable salt thereof. Particular cancers for treatment or prevention include bladder, head and neck, breast, stomach, ovary, colon, lung, brain, larynx, lymphatic system, liver, skin, hematopoetic system, genitourinary tract, gastrointestinal, ovarian, prostate, gastric, bone, small-cell lung, glioma, colorectal and pancreatic cancers. More particularly, the invention relates to a method of treating or preventing gastric cancer or colorectal cancer in a mammal in need of such treatment, wherein the method comprises administering to said mammal a therapeutically effective amount of a compound of Formula I, a stereoisomer, tautomer, prodrug or pharmaceutically acceptable salt thereof. Another embodiment includes a method of treating or preventing neurodegenerative disease in a mammal in need of such treatment, wherein the method comprises administering to said mammal a therapeutically effective amount of a compound of Formula I, a stereoisomer, tautomer, prodrug or pharmaceutically acceptable salt thereof. Particular neurodegenerative disease for treatment includes Alzhemers disease, parkinson's disease, Huntington's dsease and Amyotrophic lateral sclerosis (ALS).

### Combination Therapy

The compounds of the invention can be used in combination with small molecule inhibitors such as tyrosine kinase inhibitors, Serine/Threonine kinase inhibitors, lipid kinase inhibitors, protein-protein inhibitors, etc., cytotoxic agents, radiotherapy, antibodies and cancer vaccines for the treatment of cancer.

### EXAMPLES

The invention will be more fully understood by reference to the following examples. They should not, however, be construed as limiting the scope of the invention.

Abbreviations used herein are as follows:
μL: microliter
μm: micrometer
μM: micromoles per liter
AcOK: potassium acetate
AcOH: acetic acid
Ar: argon
BSA: bovine serum albumin
CCK-8: Cell Counting Kit-8
DCM: dichloromethane
DEAD: diethyl azodicarboxylate
DIPEA: N,N-diisopropylethylamine
DME: 1,2-dimethoxyethane
DMF: dimethylformamide
DMSO-d6: deuterated dimethylsulfoxide
DTT: dithiothreitol
EtOAc: ethyl acetate
EGTA: ethylene glycol tetraacetic acid
g: gram
IC₅₀: the half maximal inhibitory concentration
HATU: 2-(7-aza-1H-benzotriazole-1-yl)-1,1,3,3-tetramethyluronium hexafluorophosphate
HCMV: human cytomegalovirus
HIV: human immunodeficiency
HSV: herpes simplex virus
HPV: human papillomavirus
HPLC: high performance liquid chromatography
LC/MS: Liquid chromatography/mass spectrometry
MeOH: methanol
METHANOL-d₄: perdeuteromethanol
M: molarity
mg: milligram
MHz: megahertz
min: minute
mins: minutes
mL: milliliter
mM: millimoles per liter
mm: millimeter
mmol: millimole
MS (ESI): mass spectroscopy (electron spray ionization)
nM: nanomoles per liter
nm: nanometer
NMR: nuclear magnetic resonance
N₂: nitrogen
obsd.: observed
OD: optical density
Pd(PPh₃)₄: tetrakis(triphenylphosphine)palladium
Pd(PPh₃)₂Cl₂: bis(triphenylphosphine)palladium(II) chloride
Pd(dppf)Cl₂: [1,1′-bis(diphenylphosphino)ferrocene]dichloropalladium(II)
PE or Pet: petroleum ether
Prep HPLC: preparative high performance liquid chromatography
SFC: supercritical fluid chromatography
TEA: triethylamine
THF: tetrahydrofurane
TLC: thin layer chromatography
TR-FRET: time resolved-fluorescence resonance energy transfer
δ: chemical shift

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μM; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using a MicroMass Plateform LC (Waters™ alliance 2795-ZQ2000). Standard LC/MS conditions were as follows (running time 6 minutes): Acidic condition: A: 0.1% formic acid in H₂O; B: 0.1% formic acid in acetonitrile; Basic condition: A: 0.01% NH₃.H₂O in H₂O; B: acetonitrile; Neutral condition: A: H₂O; B: acetonitrile.

Mass spectra (MS): generally only ions which indicate the parent mass are reported, and unless otherwise stated the mass ion quoted is the positive mass ion (M+H)⁺.

The microwave assisted reactions were carried out in a Biotage Initiator Sixty.

NMR Spectra were obtained using Bruker Avance 400 MHz.

All reactions involving air-sensitive reagents were performed under an argon atmosphere. Reagents were used as received from commercial suppliers without further purification unless otherwise noted.

The following examples were prepared by the general methods outlined in the schemes above. They are intended to illustrate the meaning of the present invention but should by no means represent a limitation within the meaning of the present invention.

### PREPARATIVE EXAMPLES

### Example 1

### Preparation of 5-[5-((R)-2-hydroxy-1-phenyl-ethylamino)-pyridin-3-yl]-1,3-dihydro-indol-2-one

### Step 1: Preparation of (R)-2-(5-bromo-pyridin-3-ylamino)-2-phenyl-ethanol

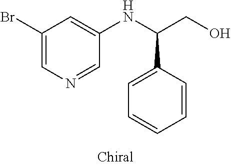 [SMILES: OC[C@H](Nc1cncc(Br)c1)c1ccccc1]

Under an Ar atmosphere, a mixture of 3, 5-dibromopyridine (8 g, 33.8 mmol), D-(−)-alpha-phenylglycinol (7.41 g, 54 mmol), Copper(I) iodide (0.64 g, 3.38 mmol), L-proline (0.78 g, 6.7 mmol) and potassium carbonate (9.32 g, 67 mmol) in DMSO (200 mL) was heated at 100° C. for 12 hours. The mixture was diluted with H₂O and then filtered to remove the catalyst. The aqueous layer was extracted with EtOAc, and the organic layer was washed with brine, and then dried over Na₂SO₄ and then concentrated. The residue was purified by chromatography column to give (R)-2-(5-bromo-pyridin-3-ylamino)-2-phenyl-ethanol (8.9 g) as a yellow solid.

### Step 2: Preparation of (R)-5-(2-hydroxy-1-phenylethyl)-amino)-pyridin-3-yl)-boronic acid

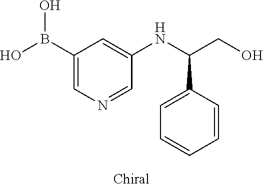 [SMILES: OC[C@H](Nc1cncc(B(O)O)c1)c1ccccc1]

Under an Ar atmosphere, a mixture of (R)-2-(5-bromo-pyridin-3-ylamino)-2-phenyl-ethanol (3 g, 10.27 mmol), bis(pinacolato)diboron (5.22 g, 20.55 mmol), [1,1-bis(diphenylphosphino)ferrocene]dichloropalladium(II) (0.84 g, 1.027 mmol) and AcOK (2.01 g, 20.5 mmol) in 1,4-dioxane was exposed to microwave irradiation at 120° C. for 2 hours. Then saturated Na₂CO₃ aqueous solution was added and the reaction mixture was heated at 100° C. for 1 hour. After cooling down to room temperature, the aqueous layer was extracted with EtOAc twice and then adjusted to PH 3.0-4.0 by adding 6N HCl solution slowly. The aqueous layer was then extracted with EtOAc, and then the combined organic layers were dried and concentrated. The residue was dissolved in ether and stirred overnight. The precipitate was collected to give (R)-5-(2-hydroxy-1-phenylethyl)-amino)-pyridin-3-yl)-boronic acid (1 g).

### Step 3: Preparation of 5-[5-((R)-2-hydroxy-1-phenyl-ethylamino)-pyridin-3-yl]-1,3-dihydro-indol-2-one

Under an Ar atmosphere, a mixture of (R)-5-(2-hydroxy-1-phenylethyl)-amino)-pyridin-3-yl)-boronic acid (50 mg, 0.2 mmol), 5-bromooxindole (45 mg, 0.2 mmol), tetrakis(triphenylphosphine)palladium (12 mg) and potassium carbonate (26 mg, 0.4 mmol) in DME/H₂O (5:1, 2 mL) was heated at 90° C. under microwave for 40 mins. Then the residue was partitioned between EtOAc and brine. The aqueous layer was separated and extracted with EtOAc. The combined organic layers were concentrated and the residue was purified by Prep-HPLC to afford 5-[5-((R)-2-hydroxy-1-phenyl-ethylamino)-pyridin-3-yl]-1,3-dihydro-indol-2-one (12 mg).

### Example 2

### Preparation of 5-[5-((R)-2-hydroxy-1-phenyl-ethylamino)-pyridin-3-yl]-3,3-dimethyl-1,3-dihydro-indol-2-one

Under an Ar atmosphere, a mixture of (R)-5-(2-hydroxy-1-phenylethyl)-amino)-pyridin-3-yl)-boronic acid (100 mg, 0.4 mmol), 5-bromo-3,3-dimethyl-1,3-dihydro-indol-2-one (93 mg, 0.4 mmol), tetrakis(triphenylphosphine)palladium (25 mg) and potassium carbonate (110 mg, 0.8 mmol) in DME/H₂O (5:1, 5 mL) was heated at 90° C. under microwave for 40 mins. Then the residue was partitioned between EtOAc and brine. The aqueous layer was separated and extracted with EtOAc. The combined organic layers were concentrated and the residue was purified by Prep-HPLC to afford 5-[5-((R)-2-hydroxy-1-phenyl-ethylamino)-pyridin-3-yl]-3,3-dimethyl-1,3-dihydro-indol-2-one (21 mg).

### Example 3

### Preparation of 5-{5-[(R)-1-(2-chloro-phenyl)-2-hydroxy-ethylamino]-pyridin-3-yl}-1,3-dihydro-indol-2-one

### Step 1: Preparation of oxindole-5-boronic acid pinacol ester

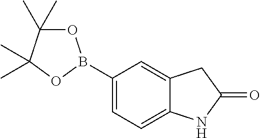 [SMILES: CC1(C)OB(c2ccc3c(c2)CC(=O)N3)OC1(C)C]

To a solution of 5-bromo-2-oxindole (21 g, 100 mmol) in 1,4-dioxane (150 mL) was added bis(pinacolato)diboron (38 g, 150 mmol), 1,1-bis(diphenylphosphino) ferrocene-palladium(II) dichloride dichloromethane complex (8.2 g, 10 mmol), and potassium acetate (20 g, 200 mmol).The resulting mixture was degassed and then stirred overnight at 80° C. under an Ar atmosphere. After the reaction was completed as monitored by LC-MS, the mixture was diluted with water (500 mL) and then extracted with EtOAc. The combined organic layers were washed with water and brine, and then dried. The solvent was concentrated and the residue was purified by column chromatography (EtOAc/Pet=2:1) to give oxindole-5-boronic acid pinacol ester (15 g) as a white solid.

### Step 2: Preparation of 5-{5-[(R)-1-(2-chloro-phenyl)-2-hydroxy-ethylamino]-pyridin-3-yl}-1,3-dihydro-indol-2-one

To a solution of (R)-2-(5-bromo-pyridin-3-ylamino)-2-(2-chloro-phenyl)-ethanol (327 mg, 1.0 mmol) in DME/H₂O (5:1, 12 mL) was added Pd(PPh₃)₄ (230 mg, 0.2 mmol), K₂CO₃ (276 mg, 2.0 mmol) and oxindole-5-boronic acid pinacol ester (310 mg, 1.2 mmol). The resulting mixture was degassed and then stirred for 10 hours at 95° C. under an Ar atmosphere. After cooling, the mixture was diluted with water (50 mL) and then extracted with EtOAc (2×75 mL). The combined organic layers were washed with water and brine, and then dried. The solvent was concentrated and the residue was purified by Prep-HPLC to give 5-{5-[(R)-1-(2-chloro-phenyl)-2-hydroxy-ethylamino]-pyridin-3-yl}-1,3-dihydro-indol-2-one (5 mg).

### Example 4

### Preparation of 5-{5-[(S)-1-(2-chloro-phenyl)-2-hydroxy-ethylamino]-pyridin-3-yl}-1,3-dihydro-indol-2-one

To a solution of (S)-2-(5-bromo-pyridin-3-ylamino)-2-(2-chloro-phenyl)-ethanol (327 mg, 1.0 mmol) in DME/H₂O (5:1, 12 mL) was added Pd(PPh₃)₄ (230 mg, 0.2 mmol), K₂CO₃ (276 mg, 2.0 mmol) and oxindole-5-boronic acid pinacol ester (310 mg, 1.2 mmol). The resulting mixture was degassed and then stirred for 10 hours at 95° C. under an Ar atmosphere. After cooling, the mixture was diluted with water (50 mL) and then extracted with EtOAc (2×75 mL). The combined organic layers were washed with water and brine, and then dried. The solvent was concentrated and the residue was purified by Prep-HPLC to give 5-{5-[(S)-1-(2-chloro-phenyl)-2-hydroxy-ethylamino]-pyridin-3-yl}-1,3-dihydro-indol-2-one (5 mg).

### Example 5

### Preparation of 5-[5-((R)-2-hydroxy-1-phenyl-ethylamino)-pyridin-3-yl]-1,3-dihydro-benzoimidazol-2-one

Under an Ar atmosphere, a mixture of (R)-2-(5-bromo-pyridin-3-ylamino)-2-phenyl-ethanol (300 mg, 1.027 mmol), 5-(4,4,5,5-tetramethyl-[1,3,2]dioxaborolan-2-yl)-1,3-dihydro-benzoimidazol-2-one (320.67 mg, 1.23 mmol), tetrakis(triphenylphosphine)palladium (237 mg, 0.205 mmol) and potassium carbonate (283 mg, 2.05 mmol) in DME/H₂O (5:1, 4.5 mL) was exposed to microwave irradiation at 100° C. for 1 hour, then the reaction mixture was concentrated in vacuo. The residue was partitioned between EtOAc and brine. The aqueous layer was separated and extracted with EtOAc. The combined organic layers were concentrated and the residue was purified by Prep-HPLC to give 5-[5-((R)-2-hydroxy-1-phenyl-ethylamino)-pyridin-3-yl]-1,3-dihydro-benzoimidazol-2-one (3 mg).

### Example 6

### Preparation of (R)-5′-(5-((2-hydroxy-1-phenylethyl)amino)pyridin-3-yl)-Spiro[cyclopropane-1,3′-indolin]-2′-one

### Step 1: Preparation of spiro(cyclopropane-1,3-indolin)-2-one-5-boronic acid pinacol ester

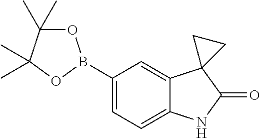 [SMILES: CC1(C)OB(c2ccc3c(c2)C2(CC2)C(=O)N3)OC1(C)C]

To a solution of 5-bromospiro(cyclopropane-1,3-indolin)-2-one (2.37 g, 10 mmol) in 1,4-dioxane (25 mL) was added bis(pinacolato)diboron (3.8 g, 15 mmol), 1,1-bis(diphenylphosphino)ferrocene-palladium(II) dichloride dichloromethane complex (0.82 g, 1 mmol), and potassium acetate (2 g, 200 mmol). The resulting mixture was degassed and then stirred overnight at 80° C. under an Ar atmosphere. After the reaction was completed as monitored by LC-MS, the mixture was diluted with water (500 mL) and then extracted with EtOAc. The combined organic layers were washed with water and brine, and then dried. The solvent was concentrated and the residue was purified by column chromatography (EtOAc/PE=2:1) to give spiro(cyclopropane-1,3-indolin)-2-one-5-boronic acid pinacol ester (1.2 g).

### Step 2: Preparation of (R)-5′-(5-((2-hydroxy-1-phenylethyl)amino)pyridin-3-yl)-spiro[cyclopropane-1,3′-indolin]-2′-one

To a solution of (R)-2-(5-bromo-pyridin-3-ylamino)-2-phenyl-ethanol (292 mg, 1.0 mmol) in DME/H₂O (5:1, 12 mL) was added Pd(PPh₃)₄ (230 mg, 0.2 mmol), K₂CO₃ (276 mg, 2.0 mmol) and spiro(cyclopropane-1,3-indolin)-2-one-5-boronic acid pinacol ester (313 mg, 1.2 mmol). The resulting mixture was degassed and then stirred for 10 hours at 95° C. under an Ar atmosphere. After cooling, the mixture was diluted with water (50 mL) and then extracted with EtOAc (2×75 mL). The combined organic layers were washed with water and brine, and then dried. The solvent was concentrated and the residue was purified by Prep-HPLC to give (R)-5′-(5-((2-hydroxy-1-phenylethyl)amino)pyridin-3-yl)-spiro[cyclopropane-1,3′-indolin]-2′-one (20 mg).

### Example 7

### Preparation of 5-[5-((R)-2-hydroxy-1-phenyl-ethylamino)-pyridin-3-yl]-1,3-dihydro-pyrrolo[2,3-b]pyridin-2-one

### Step 1: Preparation of 5-(4,4,5,5-tetramethyl-[1,3,2]dioxaborolan-2-yl)-1,3-dihydro-pyrrolo[2,3-b]pyridin-2-one

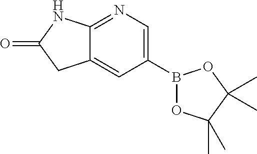 [SMILES: CC1(C)OB(c2cnc3c(c2)CC(=O)N3)OC1(C)C]

Under an Ar atmosphere, a mixture of 5-bromo-1,3-dihydro-pyrrolo[2,3-b]pyridin-2-one (3 g, 14.08 mmol), bis(pinacolato)diboron (5.37 g, 21.1 mmol), 1,1′-bis(diphenylphosphino)ferrocenedichloropalladium(II) (0.206 g, 0.28 mmol) and AcOK (4.14 g, 42 mmol) in DMF was heated at 100° C. overnight. After cooling down to the room temperature, the mixture was poured into water. The aqueous layer was extracted with EtOAc, then dried and concentrated. The residue was purified by flash column to give 5-(4,4,5,5-tetramethyl-[1,3,2]dioxaborolan-2-yl)-1,3-dihydro-pyrrolo[2,3-b]pyridin-2-one (400 mg) as a yellow solid.

### Step 2: Preparation of 5-[5-((R)-2-hydroxy-1-phenyl-ethylamino)-pyridin-3-yl]-1,3-dihydro-pyrrolo[2,3-b]pyridin-2-one

Under an Ar atmosphere, a mixture of (R)-2-(5-bromo-pyridin-3-ylamino)-2-phenyl-ethanol (480 mg, 1.644 mmol), 5-(4,4,5,5-tetramethyl-[1,3,2]dioxaborolan-2-yl)-1,3-dihydro-pyrrolo[2,3-b]pyridin-2-one (468 mg, 1.81 mmol), bis(triphenylphosphine)palladium(II) chloride (232 mg, 0.328 mmol) and potassium carbonate (452 mg, 3.28 mmol) in DMF/H₂O (5:1, 10 mL) was exposed to microwave irradiation at 105° C. for 1 hour. Then the mixture was diluted with EtOAc. The aqueous layer was extracted with EtOAc. The combined organic layers were concentrated and the residue was purified by Prep-HPLC to give 5-[5-((R)-2-hydroxy-1-phenyl-ethylamino)-pyridin-3-yl]-1,3-dihydro-pyrrolo[2,3-b]pyridin-2-one (6.5 mg).

### Example 8

### Preparation of 3,3-difluoro-5-[5-((R)-2-hydroxy-1-phenyl-ethylamino)-pyridin-3-yl]-1,3-dihydro-indol-2-one

### Step 1: Preparation of 3,3-difluoro-5-(4,4,5,5-tetramethyl-[1,3,2]dioxaborolan-2-yl)-1,3-dihydro-indol-2-one

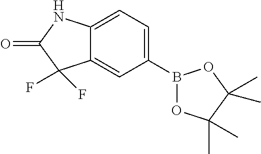 [SMILES: CC1(C)OB(c2ccc3c(c2)C(F)(F)C(=O)N3)OC1(C)C]

Under an Ar atmosphere, a mixture of 5-bromo-3,3-difluoro-1,3-dihydro-indol-2-one (1 g, 4.05 mmol), bis(pinacolato)diboron (2.05 g, 8.06 mmol), 1,1′-bis(diphenylphosphino)ferrocenedichloropalladium(II) (0.331 g, 0.403 mmol) and AcOK (1.18 g, 12.09 mmol) in 1,4-dioxane was heated at 95° C. overnight. After cooling down to the room temperature, the mixture was concentrated and the residue was purified by flash column to give 3,3-difluoro-5-(4,4,5,5-tetramethyl-[1,3,2]dioxaborolan-2-yl)-1,3-dihydro-indol-2-one (600 mg).

### Step 2: Preparation of 3,3-difluoro-5-[5-((R)-2-hydroxy-1-phenyl-ethylamino)-pyridin-3-yl]-1,3-dihydro-indol-2-one

Under an Ar atmosphere, a mixture of (R)-2-(5-bromo-pyridin-3-ylamino)-2-phenyl-ethanol (300 mg, 1.027 mmol), 3,3-difluoro-5-(4,4,5,5-tetramethyl-[1,3,2]dioxaborolan-2-yl)-1,3-dihydro-indol-2-one (303 mg, 1.027 mmol), bis(triphenylphosphine)palladium(II) chloride (144 mg, 0.205 mmol) and potassium carbonate (425 mg, 3.08 mmol) in DMF/H₂O (5:1, 10 mL) was exposed to microwave irradiation at 105° C. for 1 hour. Then the mixture was diluted with EtOAc. The aqueous layer was extracted with EtOAc. The combined organic layers were concentrated and the residue was purified by Prep-HPLC to give 3,3-difluoro-5-[5-((R)-2-hydroxy-1-phenyl-ethylamino)-pyridin-3-yl]-1,3-dihydro-indol-2-one (14 mg).

### Example 9

### Preparation of 5-[5-((R)-2-hydroxy-1-phenyl-ethylamino)-pyridin-3-yl]-1,3-dihydro-pyrrolo[3,2-b]pyridin-2-one

### Step 1: Preparation of (2-oxo-2,3-dihydro-1H-pyrrolo[3,2-b]pyridine-5-yl)boronic acid

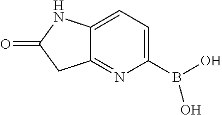 [SMILES: O=C1Cc2nc(B(O)O)ccc2N1]

Under an Ar atmosphere, a mixture of 5-bromo-1,3-dihydro-pyrrolo[3,2-b]pyridin-2-one (300 mg, 1.41 mmol), bis(pinacolato)diboron (358 mg, 1.41 mmol), [1,1′-bis(diphenylphosphino)ferrocene]dichloropalladium(II) (230 mg, 0.28 mmol) and AcOK (276 mg, 2.82 mmol) in 1,4-dioxane was heated at 95° C. overnight. After cooling down to the room temperature, the mixture was concentrated and the residue was used in the next step without further purification.

### Step 2: Preparation of 5-[5-((R)-2-hydroxy-1-phenyl-ethylamino)-pyridin-3-yl]-1,3-dihydro-pyrrolo[3,2-b]pyridin-2-one

Under an Ar atmosphere, a mixture of crude (2-oxo-2,3-dihydro-1H-pyrrolo[3,2-b]pyridine-5-yl)boronic acid (273 mg), (R)-2-(5-bromo-pyridin-3-ylamino)-2-phenyl-ethanol (535 mg, 1.831 mmol), bis(triphenylphosphine)palladium(II) chloride (198 mg, 0.28 mmol) and potassium carbonate (583 mg, 4.21 mmol) in DMF/H₂O (5:1, 10 mL) was exposed to microwave irradiation at 100° C. for 1 hour. Then the mixture was diluted with EtOAc. The aqueous layer was extracted with EtOAc. The combined organic layers were concentrated and the residue was purified by Prep-HPLC to give 5-[5-((R)-2-hydroxy-1-phenyl-ethylamino)-pyridin-3-yl]-1,3-dihydro-pyrrolo[3,2-b]pyridin-2-one (6 mg).

### Example 10

### Preparation of 6-fluoro-5-[5-((R)-2-hydroxy-1-phenyl-ethylamino)-pyridin-3-yl]-1,3-dihydro-indol-2-one

To a solution of (R)-2-(5-bromo-pyridin-3-ylamino)-2-phenyl-ethanol (292 mg, 1.0 mmol) in DME/H₂O (5:1, 12 mL) was added Pd(PPh₃)₄ (230 mg, 0.2 mmol), K₂CO₃ (276 mg, 2.0 mmol) and 6-fluoro-5-(4,4,5,5-tetramethyl-[1,3,2]dioxaborolan-2-yl)-1,3-dihydro-indol-2-one (277 mg, 1.0 mmol). The resulting mixture was degassed and then stirred for 10 hours at 95° C. under an Ar atmosphere. After cooling, the mixture was diluted with water (50 mL) and then extracted with EtOAc (2×75 mL). The combined organic layers were washed with water and brine, dried, and then concentrated. The residue was purified by Prep-HPLC to give 6-fluoro-5-[5-((R)-2-hydroxy-1-phenyl-ethylamino)-pyridin-3-yl]-1,3-dihydro-indol-2-one (30 mg).

### Example 11

### Preparation of 7-fluoro-5-[5-((R)-2-hydroxy-1-phenyl-ethylamino)-pyridin-3-yl]-1,3-dihydro-indol-2-one

To a solution of (R)-2-(5-bromo-pyridin-3-ylamino)-2-phenyl-ethanol (292 mg, 1.0 mmol) in DME/H₂O (5:1, 12 mL) was added Pd(PPh₃)₄ (230 mg, 0.2 mmol), K₂CO₃ (276 mg, 2.0 mmol) and 7-fluoro-5-(4,4,5,5-tetramethyl-[1,3,2]dioxaborolan-2-yl)-1,3-dihydro-indol-2-one (277 mg, 1.0 mmol). The resulting mixture was degassed and then stirred for 10 hours at 95° C. under an Ar atmosphere. After cooling, the mixture was diluted with water (50 mL) and then extracted with EtOAc (2×75 mL). The combined organic layers were washed with water and brine, and then dried. The solvent was concentrated and the residue was purified by Prep-HPLC to give 7-fluoro-5-[5-((R)-2-hydroxy-1-phenyl-ethylamino)-pyridin-3-yl]-1,3-dihydro-indol-2-one (5 mg).

### Example 12

### Preparation of 6-[5-((R)-2-hydroxy-1-phenyl-ethylamino)-pyridin-3-yl]-3H-benzooxazol-2-one

6-Bromo-3H-benzooxazol-2-one (428 mg, 2.0 mmol), bis(pinacolato)diboron (508 mg, 2.0 mmol), tris(dibenzylideneacetone)dipalladium (55 mg, 0.06 mmol), butyldi-1-adamantylphosphine (65 mg, 0.18 mmol), potassium acetate (588 mg, 6.0 mmol) were added into a 10 mL microwave vial containing a magnetic stirrer bar, followed by isopropyl acetate (1.5 mL). The vessel was sealed with a cap under an argon atmosphere, then the resulting mixture was heated to 83° C. for 1 hour. After the reaction was completed as monitored by TLC and LC-MS, (R)-2-(5-bromo-pyridin-3-ylamino)-2-phenyl-acetamide (467 mg, 1.6 mmol), potassium carbonate (662 mg, 4.8 mmol), isopropyl acetate (2 mL) and H₂O (0.5 mL) were added into the above mixture. The vessel was sealed with a cap under an argon atmosphere, and then the reaction mixture was heated to 90° C. for 40 mins under microwave. The mixture was cooled to room temperature and diluted with water (25 mL), extracted with ethyl acetate (50 mL×3). The combined organic layers were washed with brine (30 mL), and then dried over anhydrous sodium sulfate, concentrated to give crude title compound. The crude title compound was purified by Prep-HPLC to give 6-[5-((R)-2-hydroxy-1-phenyl-ethylamino)-pyridin-3-yl]-3H-benzooxazol-2-one (15 mg).

### Example 13

### Preparation of 6-[5-((R)-2-hydroxy-1-phenyl-ethylamino)-pyridin-3-yl]-3H-benzothiazol-2-one

### Step 1: Preparation of 6-(4,4,5,5-tetramethyl-[1,3,2]dioxaborolan-2-yl)-3H-benzothiazol-2-one

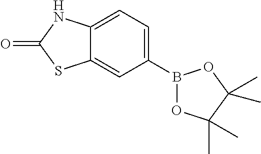 [SMILES: CC1(C)OB(c2ccc3[nH]c(=O)sc3c2)OC1(C)C]

Under an Ar atmosphere, a mixture of 6-bromo-3H-benzothiazol-2-one (1 g, 4.35 mmol), bis(pinacolato)diboron (1.1 g, 4.35 mmol), tris(dibenzylideneacetone)dipalladium(0) (0.119 g, 0.013 mmol), butyldi-1-adamantylphosphine (0.14 g, 0.039 mmol) and AcOK (1.28 g, 1.30 mmol) in DME was heated at 65° C. overnight. After cooling down to the room temperature, the mixture was concentrated and the residue was purified by flash column to give 6-(4,4,5,5-tetramethyl-[1,3,2]dioxaborolan-2-yl)-3H-benzothiazol-2-one (300 mg).

### Step 2: Preparation of 6-[5-((R)-2-hydroxy-1-phenyl-ethylamino)-pyridin-3-yl]-3H-benzothiazol-2-one

Under an Ar atmosphere, a mixture of (R)-2-(5-bromo-pyridin-3-ylamino)-2-phenyl-ethanol (300 mg, 1.027 mmol), 6-(4,4,5,5-tetramethyl-[1,3,2]dioxaborolan-2-yl)-3H-benzothiazol-2-one (284 mg, 1.027 mmol), tetrakis(triphenylphosphine)palladium (59 mg, 0.051 mmol) and potassium carbonate (425 mg, 3.08 mmol) in DME/H₂O (5:1, 10 mL) was exposed to microwave irradiation at 100° C. for 5 hours, then concentrated in vacuo. The residue was partitioned between EtOAc and brine. The aqueous layer was separated and extracted with EtOAc. The combined organic layers were concentrated and the residue was purified by Prep-HPLC to give 6-[5-((R)-2-hydroxy-1-phenyl-ethylamino)-pyridin-3-yl]-3H-benzothiazol-2-one (39 mg).

### Example 14

### Preparation of (R)-2-(5-benzo[1,3]dioxol-5-yl-pyridin-3-ylamino)-2-phenyl-ethanol

(R)-2-(5-Bromo-pyridin-3-ylamino)-2-phenyl-ethanol (59 mg, 0.2 mmol), 3,4-methylenedioxphenylboronic acid (43 mg, 0.26 mmol), tetrakis(triphenylphosphine) palladium (11 mg, 0.01 mmol) and potassium carbonate (81 mg, 0.6 mmol) were added into a 10 mL microwave vial containing a magnetic stirrer bar, followed by DME (1 mL) and H₂O (0.2 mL). The vessel was sealed with a cap under an argon atmosphere, and then the resulting mixture was heated to 90° C. for 40 minutes under microwave. The mixture was cooled to room temperature and diluted with water (5 mL), extracted with ethyl acetate (10 mL×3). The combined organic layers were washed with brine (10 mL), dried over anhydrous sodium sulfate, concentrated in vacuo to give crude title compound. The crude title compound was purified by C-18 reversed phase HPLC column to give (R)-2-(5-benzo[1,3]dioxol-5-yl-pyridin-3-ylamino)-2-phenyl-ethanol (20 mg) as a white solid.

### Example 15

### Preparation of (R)-2-[5-(2,3-dihydro-benzo[1,4]dioxin-6-yl)-pyridin-3-ylamino]-2-phenyl-ethanol

(R)-2-(5-Bromo-pyridin-3-ylamino)-2-phenyl-ethanol (59 mg, 0.2 mmol), 1,4-benzodioxane-6-boronic acid (47 mg, 0.26 mmol), tetrakis(triphenylphosphine) palladium (11 mg, 0.01 mmol) and potassium carbonate (81 mg, 0.6 mmol) were added into a 10 mL microwave vial containing a magnetic stirrer bar, followed by DME (1 mL) and H₂O (0.2 mL). The vessel was sealed with a cap under an argon atmosphere, then the resulting mixture was heated to 90° C. for 40 minutes under microwave. The mixture was cooled to room temperature and diluted with water (5 mL), extracted with ethyl acetate (10 mL×3). Then the combined organic layers were washed with brine (10 mL), dried over anhydrous sodium sulfate, concentrated in vacuo to give crude title compound. The crude title compound was purified by C-18 reversed phase HPLC column to give (R)-2-[5-(2,3-dihydro-benzo[1,4]dioxin-6-yl)-pyridin-3-ylamino]-2-phenyl-ethanol (25 mg) as a white solid.

### Example 16

### Preparation of (R)-2-[5-(1H-indazol-5-yl)-pyridin-3-ylamino]-2-phenyl-ethanol

Under an Ar atmosphere, a mixture of (R)-5-(2-hydroxy-1-phenylethyl)-amino)-pyridin-3-yl)-boronic acid (50 mg, 0.2 mmol), 5-bromo-1H-indazole (65 mg, 0.2 mmol), tetrakis(triphenylphosphine)palladium (25 mg) and potassium carbonate (70 mg, 0.5 mmol) in DME/H₂O (5:1, 5 mL) was heated at 90° C. under microwave for 40 mins. Then the residue was partitioned between EtOAc and brine. The aqueous layer was separated and extracted with EtOAc. The combined organic layers were concentrated and the residue was purified by Prep-HPLC to afford (R)-2-[5-(1H-indazol-5-yl)-pyridin-3-ylamino]-2-phenyl-ethanol (4 mg).

### Example 17

### Preparation of (R)-2-[5-(1H-indol-4-yl)-pyridin-3-ylamino]-2-phenyl-ethanol

Under an Ar atmosphere, a mixture of (R)-5-(2-hydroxy-1-phenylethyl)-amino)-pyridin-3-yl)-boronic acid (50 mg, 0.2 mmol), 4-bromoindole (40 mg, 0.2 mmol), tetrakis(triphenylphosphine)palladium (12 mg) and potassium carbonate (26 mg, 0.4 mmol) in DME/H₂O (5:1, 2 mL) was heated at 90° C. under microwave for 40 mins. Then the residue was partitioned between EtOAc and brine. The aqueous layer was separated and extracted with EtOAc. The combined organic layers were concentrated and the residue was purified by Prep-HPLC to get (R)-2-[5-(1H-indol-4-yl)-pyridin-3-ylamino]-2-phenyl-ethanol (9 mg).

### Example 18

### Preparation of (R)-2-[5-(3-amino-1H-indazol-5-yl)-pyridin-3-ylamino]-2-phenyl-ethanol

Under an Ar atmosphere, a mixture of (R)-5-(2-hydroxy-1-phenylethyl)-amino)-pyridin-3-yl)-boronic acid (100 mg, 0.387 mmol), 5-bromo-1H-indazol-3-ylamine (82 mg, 0.387 mmol), tetrakis(triphenylphosphine)palladium (22 mg, 0.019 mmol) and potassium carbonate (160 mg, 1.16 mmol) in DME/H₂O (5:1, 4.5 mL) was exposed to microwave irradiation at 105° C. for 40 mins, then the reaction mixture was concentrated in vacuo. The residue was purified by Prep-HPLC to give (R)-2-[5-(3-amino-1H-indazol-5-yl)-pyridin-3-ylamino]-2-phenyl-ethanol (3 mg).

### Example 19

### Preparation of (R)-2-[5-(3-fluoro-1H-indazol-5-yl-pyridin-3-ylamino]-2-phenyl-ethanol

### Step 1: Preparation of 3-fluoro-5-(4,4,5,5-tetramethyl-[1,3,2]dioxaborolan-2-yl)-1H-indazole

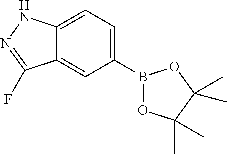 [SMILES: CC1(C)OB(c2ccc3[nH]nc(F)c3c2)OC1(C)C]

Under an Ar atmosphere, a mixture of 5-bromo-3-fluoro-1H-indazole (250 mg, 1.168 mmol), bis(pinacolato)diboron (593 mg, 2.336 mmol), [1,1-bis(diphenylphosphino)ferrocene]dichloropalladium(II) (95 mg, 0.117 mmol) and AcOK (229 mg, 2.336 mmol) in 1,4-dioxane was heated at 95° C. overnight. After cooling down to the room temperature, the mixture was concentrated and the residue was purified by flash column to give 3-fluoro-5-(4,4,5,5-tetramethyl-[1,3,2]dioxaborolan-2-yl)-1H-indazole (280 mg) as a white solid.

### Step 2: Preparation of (R)-2-[5-(3-fluoro-1H-indazol-5-yl-pyridin-3-ylamino]-2-phenyl-ethanol

Under an Ar atmosphere, a mixture of (R)-2-(5-bromo-pyridin-3-ylamino)-2-phenyl-ethanol (100 mg, 0.342 mmol), 3-fluoro-5-(4,4,5,5-tetramethyl-[1,3,2]dioxaborolan-2-yl)-1H-indazole (99 mg, 0.377 mmol), tetrakis(triphenylphosphine)palladium (79 mg, 0.068 mmol) and potassium carbonate (94 mg, 0.68 mmol) in DME/H₂O (5:1, 4.5 mL) was exposed to microwave irradiation at 100° C. for 1 hour, then the reaction mixture was concentrated in vacuo. The residue was partitioned between EtOAc and brine. The aqueous layer was separated and extracted with EtOAc. The combined organic layers were concentrated and the residue was purified by Prep-HPLC to give (R)-2-[5-(3-fluoro-1H-indazol-5-yl-pyridin-3-ylamino]-2-phenyl-ethanol (24 mg).

### Example 20

### Preparation of (R)-2-[5-(3-methyl-1H-indazol-5-yl)-pyridin-3-ylamino]-2-phenyl-ethanol

### Step 1: Preparation of 3-methyl-5-(4,4,5,5-tetramethyl-[1,3,2]dioxaborolan-2-yl)-1H-indazole

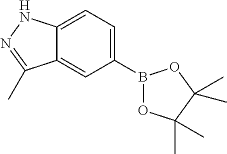 [SMILES: Cc1n[nH]c2ccc(B3OC(C)(C)C(C)(C)O3)cc12]

Under an Ar atmosphere, a mixture of 5-bromo-3-methyl-1H-indazole (3 g, 14.2 mmol), bis(pinacolato)diboron (7.2 g, 28.4 mmol), [1,1-bis(diphenylphosphino)ferrocene]dichloropalladium(II) (1.16 g, 1.42 mmol) and AcOK (2.79 g, 28.4 mmol) in 1,4-dioxane was heated at 95° C. overnight. After cooling down to the room temperature, the mixture was concentrated and the residue was purified by flash column to give 3-methyl-5-(4,4,5,5-tetramethyl-[1,3,2]dioxaborolan-2-yl)-1H-indazole (2.5 g) as yellow oil.

### Step 2: Preparation of (R)-2-[5-(3-methyl-1H-indazol-5-yl)-pyridin-3-ylamino]-2-phenyl-ethanol

Under an Ar atmosphere, a mixture of (R)-2-(5-bromo-pyridin-3-ylamino)-2-phenyl-ethanol (150 mg, 0.51 mmol), 3-methyl-5-(4,4,5,5-tetramethyl-[1,3,2]dioxaborolan-2-yl)-1H-indazole (146 mg, 0.565 mmol), tetrakis(triphenylphosphine)palladium (119 mg, 0.103 mmol) and potassium carbonate (142 mg, 1.027 mmol) in DME/H₂O (5:1, 4.5 mL) was exposed to microwave irradiation at 100° C. for 1 hour, then the reaction mixture was concentrated in vacuo. The residue was partitioned between EtOAc and brine. The aqueous layer was separated and extracted with EtOAc. The combined organic layers were concentrated and the residue was purified by Prep-HPLC to give (R)-2-[5-(3-methyl-1H-indazol-5-yl)-pyridin-3-ylamino]-2-phenyl-ethanol (20 mg).

### Example 21

### Preparation of (R)-2-[5-(3-ethyl-1H-indazol-5-yl)-pyridin-3-ylamino]-2-phenyl-ethanol

Under an Ar atmosphere, a mixture of (R)-5-(2-hydroxy-1-phenylethyl)-amino)-pyridin-3-yl)-boronic acid (100 mg, 0.387 mmol), 5-bromo-3-ethyl-1H-indazol (82 mg, 0.387 mmol), tetrakis(triphenylphosphine)palladium (22 mg, 0.019 mmol) and potassium carbonate (160 mg, 1.16 mmol) in DME/H₂O (5:1, 4.5 mL) was exposed to microwave irradiation at 105° C. for 40 mins, then the reaction mixture was concentrated in vacuo. The residue was purified by Prep-HPLC to give (R)-2-[5-(3-ethyl-1H-indazol-5-yl)-pyridin-3-ylamino]-2-phenyl-ethanol (13 mg).

### Example 22

### Preparation of (R)-2-[5-(3-methylsulfanyl-1H-indazol-5-yl)-pyridin-3-ylamino]-2-phenyl-ethanol

### Step 1: Preparation of 5-bromo-3-iodo-1H-indazo

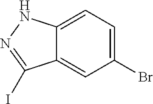 [SMILES: Brc1ccc2[nH]nc(I)c2c1]

To a solution of 5-bromoindazole 1 (2.53 g, 12.84 mmol) in DMF (20 mL) was added I₂ (3.26 g, 12.84 mmol) and KOH (1.44 g, 25.68 mmol) successively. The mixture was stirred at room temperature for 2 hours. After the reaction was completed as monitored by LC-MS, water (300 mL) was added, and then the precipitate was collected by suction to give 5-bromo-3-iodo-1H-indazo 2 (3.73 g) as a white solid.

### Step2: Preparation of 5-bromo-3-methylsulfanyl-1H-indazole

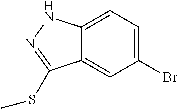

To a solution of 5-bromo-3-iodo-1H-indazo 2 (3.7 g, 11.49 mmol) in DMSO (20 mL) was added 20% aqueous MeSNa solution (2.4 mL, 34.47 mmol) and CuI (218 mg, 1.15 mmol) successively. The resulting mixture was degassed and charged with N₂. After heating at 120° C. for 3 hours, the reaction was cooled down to room temperature. Water (50 mL) was added and the mixture was extracted with ethyl acetate (50 mL×3). The combined organic layers were dried and concentrated under reduce pressure. The residue was purified by flash column to give 5-bromo-3-methylsulfanyl-1H-indazole (2.5 g) as a yellow solid.

### Step 3: Preparation of (R)-2-[5-(3-methylsulfanyl-1H-indazol-5-yl)-pyridin-3-ylamino]-2-phenyl-ethanol

A mixture of 5-bromo-3-methylsulfanyl-1H-indazole (320 mg, 1.33 mmol), bis(pinacolato)diboron (338 mg, 1.33 mmol), AcOK (260 mg, 2.66 mmol) and Pd(dppf)Cl₂ dichloromethane complex (57 mg, 0.07 mmol) in dioxane (10 mL) was degassed and charged with N₂. The reaction was heated to reflux with stirring overnight. After cooling down to room temperature, water (10 mL) was added and the mixture was extracted with ethyl acetate (30 mL×3). The combined organic layers were dried and then concentrated. The residue was used for the next step directly. To a solution of the crude boronic ester in dioxane (7 mL) and H₂O (2 mL) was added (R)-2-(5-bromo-pyridin-3-ylamino)-2-phenyl-ethanol (388 mg, 1.33 mmol) and K₂CO₃ (368 mg, 2.66 mmol). The mixture was degassed and charged with N₂. Then Pd(PPh₃)₄ (80 mg, 0.07 mmol) was added and the reaction was heated to 150° C. in microwave reactor for 2 hours. The solvent was removed and the residue was purified by flash column to give (R)-2-[5-(3-methylsulfanyl-1H-indazol-5-yl)-pyridin-3-ylamino]-2-phenyl-ethanol (160 mg).

### Example 23

### Preparation of (R)-2-[5-(3-methanesulfonyl-1H-indazol-5-yl)-pyridin-3-ylamino]-2-phenyl-ethanol

To a solution of (R)-2-[5-(3-methylsulfanyl-1H-indazol-5-yl)-pyridin-3-ylamino]-2-phenyl-ethanol (120 mg, 0.32 mmol) in DMF(10 mL) was added oxone (390 mg, 0.64 mmol). The reaction was stirred at room temperature overnight. The reaction was quenched with saturated NaHSO₃ and followed by saturated NaHCO₃ to neutralization, and then the mixture was extracted with ethyl acetate (30 mL×3). The combined organic layers were dried and concentrated in vacuo. The residue was purified by flash column to give 80 mg of (R)-2-[5-(3-methanesulfonyl-1H-indazol-5-yl)-pyridin-3-ylamino]-2-phenyl-ethanol.

### Example 24

### Preparation of (R)-2-phenyl-2-[5-(1H-pyrazolo[3,4-b]pyridin-5-yl)-pyridin-3-ylamino]-ethanol

To a solution of (R)-5-(2-hydroxy-1-phenylethyl)-amino)-pyridin-3-yl)-boronic acid (149 mg, 0.75 mmol) in DME/H₂O (5:1, 6 mL) was added Pd(PPh₃)₄ (173 mg, 0.15 mmol), K₂CO₃ (207 mg, 1.5 mmol) and 5-bromo-1H-pyrazolo[3,4-b]pyridine (193.5 mg, 0.75 mmol). The resulting mixture was degassed and then stirred for 10 hours at 95° C. under an Ar atmosphere. After cooling, the mixture was diluted with water (30 mL) and then extracted with EtOAc (2×50 mL). The combined organic layers were washed with water and brine, and then dried. The solvent was concentrated and the residue was purified by Prep-HPLC to give (R)-2-phenyl-2-[5-(1H-pyrazolo[3,4-b]pyridin-5-yl)-pyridin-3-ylamino]-ethanol (8 mg).

### Example 25

### Preparation of (R)-2-phenyl-2-[5-(1H-pyrazolo[3,4-c]pyridin-5-yl)-pyridin-3-ylamino]-ethanol

To a solution of (R)-5-(2-hydroxy-1-phenylethyl)-amino)-pyridin-3-yl)-boronic acid (149 mg, 0.75 mmol) in DME/H₂O (5:1, 6 mL) was added Pd(PPh₃)₄ (173 mg, 0.15 mmol), K₂CO₃ (207 mg, 1.5 mmol) and 5-bromo-1H-pyrazolo[3,4-c]pyridine (193.5 mg, 0.75 mmol). The resulting mixture was degassed and then stirred for 10 hours at 95° C. under an Ar atmosphere. After cooling, the mixture was diluted with water (30 mL) and then extracted with EtOAc (2×50 mL). The combined organic layers were washed with water and brine, and then dried. The solvent was concentrated and the residue was purified by Prep-HPLC to give (R)-2-phenyl-2-[5-(1H-pyrazolo[3,4-c]pyridin-5-yl)-pyridin-3-ylamino]-ethanol (8 mg).

### Example 26

### Preparation of (R)-2-[5-(1H-benzotriazol-5-yl)-pyridin-3-ylamino]-2-phenyl-ethanol

Under an Ar atmosphere, a mixture of (R)-5-(2-hydroxy-1-phenylethyl)-amino)-pyridin-3-yl)-boronic acid (100 mg, 0.5 mmol), 5-bromo-1H-benzotriazo (130 mg, 0.5 mmol), tetrakis(triphenylphosphine)palladium (30 mg) and potassium carbonate (138 mg, 1 mmol) in DME/H₂O (5:1, 5 mL) was heated at 90° C. under microwave for 40 mins. Then the residue was partitioned between EtOAc and brine. The aqueous layer was separated and extracted with EtOAc. The combined organic layers were concentrated and the residue was purified by Prep-HPLC to get (R)-2-[5-(1H-benzotriazol-5-yl)-pyridin-3-ylamino]-2-phenyl-ethanol (12 mg).

### Example 27

### Preparation of (R)-2-phenyl-2-[5-(1H-pyrazolo[4,3-b]pyridin-5-yl)-pyridin-3-ylamino]-ethanol

To a solution of (R)-5-(2-hydroxy-1-phenylethyl)-amino)-pyridin-3-yl)-boronic acid (100 mg, 0.5 mmol) in DME/H₂O (5:1, 6 mL) was added Pd(PPh₃)₄ (116 mg, 0.1 mmol), K₂CO₃ (138 mg, 1.0 mmol) and 5-bromo-1H-pyrazolo[4,3-b]pyridine (125 mg, 0.5 mmol). The resulting mixture was degassed and then stirred for 10 hours at 95° C. under an Ar atmosphere. After cooling, the mixture was diluted with water (30 mL) and then extracted with EtOAc (2×50 mL). The combined organic layers were washed with water and brine, and then dried. The solvent was concentrated and the residue was purified by Prep-HPLC to give (R)-2-phenyl-2-[5-(1H-pyrazolo[4,3-b]pyridin-5-yl)-pyridin-3-ylamino]-ethanol (5 mg).

### Example 28

### Preparation of (R)-2-[5-(3-chloro-1H-indazol-5-yl)-pyridin-3-ylamino]-2-phenyl-ethanol

### Step 1: Preparation of 3-chloro-5-(4,4,5,5-tetramethyl-[1,3,2]dioxaborolan-2-yl)-1H-indazole

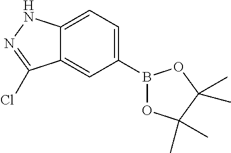 [SMILES: CC1(C)OB(c2ccc3[nH]nc(Cl)c3c2)OC1(C)C]

Under an Ar atmosphere, a mixture of 5-bromo-3-chloro-1H-indazo (1 g, 4.3 mmol), bis(pinacolato)diboron (2.2 g, 8.7 mmol), [1,1′-bis(diphenylphosphino)ferrocene]-dichloropalladium(II) (700 mg) and potassium acetate (1.26 g, 12.9 mmol) in 1,4-dioxane (12 mL) was heated at 90° C. overnight. The residue was partitioned between EtOAc and brine. The aqueous layer was separated and extracted with EtOAc. The combined organic layers were concentrated and the residue was purified by column chromatography to afford 3-chloro-5-(4,4,5,5-tetramethyl-[1,3,2]dioxaborolan-2-yl)-1H-indazole (420 mg) which was directly used in the next step.

### Step 2: Preparation of (R)-2-[5-(3-chloro-1H-indazol-5-yl)-pyridin-3-ylamino]-2-phenyl-ethanol

Under an Ar atmosphere, a mixture of (R)-2-(5-bromo-pyridin-3-ylamino)-2-phenyl-ethanol (200 mg, 0.72 mmol), 3-chloro-5-(4,4,5,5-tetramethyl-[1,3,2]dioxaborolan-2-yl)-1H-indazole (212 mg, 0.72 mmol), tetrakis(triphenylphosphine)palladium (40 mg) and potassium carbonate (200 mg, 1.44 mmol) in DME/H2O (5:1, 8 mL) was heated at 90° C. under microwave for 40 mins. Then the residue was partitioned between EtOAc and brine. The aqueous layer was separated and extracted with EtOAc. The combined organic layers were concentrated and the residue was purified by Prep-HPLC to get (R)-2-[5-(3-chloro-1H-indazol-5-yl)-pyridin-3-ylamino]-2-phenyl-ethanol (20 mg).

### Example 29

### Preparation of 2-[5-(2-oxo-2,3-dihydro-1H-indol-5-yl)-pyridin-3-ylamino]-2-phenyl-acetamide

### Step 1: Preparation of (5-bromo-pyridin-3-ylamino)-phenyl-acetic acid

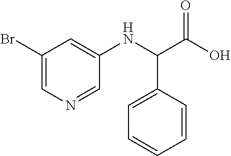 [SMILES: O=C(O)C(Nc1cncc(Br)c1)c1ccccc1]

To a solution of 3, 5-dibromopyridine (19 g, 80 mmol) in DMSO (150 mL) was added 2-phenylglycine (18 g, 120 mmol), copper(I) iodide (1.52 g, 8 mmol), L-proline (1.84 g,16 mmol) and K₂CO₃ (22 g, 160 mmol). The resulting mixture was degassed and then stirred at 90° C. for 12 hours under Ar atmosphere. After the reaction was completed as monitored by LC-MS, the mixture was diluted with water (500 mL) and then extracted with EtOAc. The combined organic layers were washed with water and brine, and then dried. The solvent was concentrated and the residue was purified by column chromatography (DCM/MeOH=20:1) to give (5-bromo-pyridin-3-ylamino)-phenyl-acetic acid (5.6 g).

### Step 2: Preparation of 2-(5-bromo-pyridin-3-ylamino)-2-phenyl-acetamide

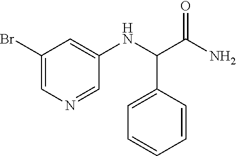 [SMILES: NC(=O)C(Nc1cncc(Br)c1)c1ccccc1]

To a solution of (5-bromo-pyridin-3-ylamino)-2-phenyl-acetic acid (2.8 g, 9.15 mmol) in anhydrous DMF (30 mL) was added 36.6 mL of NH₃ solution (0.5 M in 1, 4-dioxane) and triethylamine (1.85 g, 18.3 mmol). The resulting mixture was stirred for 30 mins, then HATU (7.0 g, 18.3 mmol) was added in batches and then the mixture was stirred overnight at room temperature. The mixture was diluted with water (200 mL) and then extracted with EtOAc (2×100 mL).The combined organic layers were washed with water and brine, and then dried. The solvent was concentrated and the residue was purified by column chromatography (DCM/MeOH=20:1) to give 2-(5-bromo-pyridin-3-ylamino)-2-phenyl-acetamide (1.2 g).

### Step 3: Preparation of (5-((2-amino-2-oxo-1-phenylethyl)amino)pyridin-3-yl)boronic acid

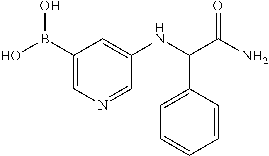 [SMILES: NC(=O)C(Nc1cncc(B(O)O)c1)c1ccccc1]

Under an Ar atmosphere, a mixture of 2-(5-bromo-pyridin-3-ylamino)-2-phenyl-acetamide (3.14 g, 10.27 mmol), bis(pinacolato)diboron (5.22 g, 20.55 mmol), [1,1-bis(diphenylphosphino)ferrocene]dichloropalladium(II) (0.84 g, 1.027 mmol) and AcOK (2.01 g, 20.5 mmol) in 1,4-dioxane was exposed to microwave irradiation at 120° C. for 1 hour. Then saturated Na₂CO₃ aqueous solution was added and the mixture was heated at 100° C. for 1 hour. After cooling down to room temperature, the aqueous layer was extracted with EtOAc twice and then adjusted to PH 3-4 by adding 6N HCl solution slowly. The aqueous layer was extracted with EtOAc, and the combined organic layers were dried and concentrated. The residue was dissolved in ether and stirred overnight. The precipitate was collected to give (5-((2-amino-2-oxo-1-phenylethyl)amino)pyridin-3-yl) boronic acid (950 mg).

### Step 4: Preparation of 2-[5-(2-oxo-2,3-dihydro-1H-indol-5-yl)-pyridin-3-ylamino]-2-phenyl-acetamide

Under an Ar atmosphere, a mixture of (5-((2-amino-2-oxo-1-phenylethyl)amino)pyridin-3-yl) boronic acid (272 mg, 1 mmol), 5-bromooxindole (212 mg, 1 mmol), tetrakis(triphenylphosphine)palladium (60 mg) and potassium carbonate (280 mg, 2 mmol) in DME/H₂O (5:1, 8 mL) was heated at 90° C. under microwave for 40 mins. Then the residue was partitioned between EtOAc and brine. The aqueous layer was separated and extracted with EtOAc. The combined organic layers were concentrated and the residue was purified by Prep-HPLC to get 2-[5-(2-oxo-2,3-dihydro-1H-indol-5-yl)-pyridin-3-ylamino]-2-phenyl-acetamide (23 mg).

### Example 30

### Preparation of N-{(R)-2-[5-(2-oxo-2,3-dihydro-1H-indol-5-yl)-pyridin-3-ylamino]-2-phenyl-ethyl}-acetamide

### Step 1: Preparation of 2-[(R)-2-(5-bromo-pyridin-3-ylamino)-2-phenyl-ethyl]-isoindole-1,3-dione

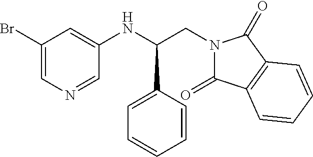 [SMILES: O=C1c2ccccc2C(=O)N1C[C@H](Nc1cncc(Br)c1)c1ccccc1]

To a solution of (R)-2-(5-bromo-pyridin-3-ylamino)-2-phenyl-ethanol (2.4 g, 8.2 mmol), phthalimide (2.78 g, 18.9 mmol) and triphenylphosphine (4.95 g, 18.9 mmol) in THF (30 mL) was added diethyl azodicarboxylate (3.29 g, 18.9 mmol) dropwise at 0° C. under argon atmosphere. The resulting mixture was stirred at room temperature overnight. The reaction mixture was diluted with water (30 mL), and then extracted with ethyl acetate (60 mL×3). The combined organic layers were washed with water (30 mL) and brine (30 mL), dried over anhydrous sodium sulfate, and concentrated in vacuo to give crude 2-[(R)-2-(5-bromo-pyridin-3-ylamino)-2-phenyl-ethyl]-isoindole-1,3-dione. The crude compound was purified by flash column to afford 2-[(R)-2-(5-bromo-pyridin-3-ylamino)-2-phenyl-ethyl]-isoindole-1,3-dione (3.01 g) as a yellow solid.

### Step 2: Preparation of (R)-N¹-(5-bromo-pyridin-3-yl)-1-phenyl-ethane-1,2-diamine

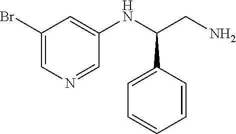 [SMILES: NC[C@H](Nc1cncc(Br)c1)c1ccccc1]

To a solution of 2-[(R)-2-(5-bromo-pyridin-3-ylamino)-2-phenyl-ethyl]-isoindole-1,3-dione (1.26 g, 3 mmol) in ethanol (10 mL) was added hydrazine hydrate (5 mL). The resulting mixture was stirred at 80° C. for 1 hour. After the reaction was completed as monitored by TLC and LC-MS, the reaction mixture was cooled to room temperature and diluted with water (20 mL), then extracted with ethyl acetate (50 mL×3). The combined organic layers were washed with water (20 mL) and brine (20 mL), dried over anhydrous sodium sulfate, concentrated in vacuo to give crude (R)-N¹-(5 -bromo-pyridin-3-yl)-1-phenyl-ethane-1,2-diamine (1.0 g) as yellow oil which was directly used for the next step without purification.

### Step 3: Preparation of N-[(R)-2-(5-bromo-pyridin-3-ylamino)-2-phenyl-ethyl]-acetamide

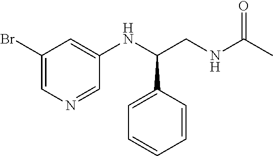 [SMILES: CC(=O)NC[C@H](Nc1cncc(Br)c1)c1ccccc1]

To a solution of crude (R)-N¹-5-bromo-pyridin-3-yl)-1-phenyl-ethane-1,2-diamine (323 mg, 1 mmol) in THF (3 mL) was added acetic anhydride (112 mg, 1.1 mmol) dropwise at 0° C. The resulting mixture was stirred at room temperature overnight. The reaction mixture was diluted with water (5 mL), and then extracted with dichloromethane (30 mL×3). The combined organic layers were washed with water (5 mL) and brine (10 mL), dried over anhydrous sodium sulfate, and concentrated in vacuo to give crude N-[(R)-2-(5-bromo-pyridin-3-ylamino)-2-phenyl-ethyl]-acetamide. The crude N-[(R)-2-(5-bromo-pyridin-3-ylamino)-2-phenyl-ethyl]-acetamide was purified by flash column to afford N-[(R)-2-(5-bromo-pyridin-3-ylamino)-2-phenyl-ethyl]-acetamide (237 mg) as a white solid.

### Step 4: Preparation of N-{(R)-2-[5-(2-oxo-2,3-dihydro-1H-indol-5-yl)-pyridin-3-ylamino]-2-phenyl-ethyl}-acetamide

N-[(R)-2-(5-bromo-pyridin-3-ylamino)-2-phenyl-ethyl]-acetamide (67 mg, 0.2 mmol), 5-(4,4,5,5-tetramethyl-[1,3,2]dioxaborolan-2-yl)-1,3-dihydro-indol-2-one (67 mg, 0.26 mmol), tetrakis(triphenylphosphine) palladium (11 mg, 0.01 mmol) and potassium carbonate (81 mg, 0.6 mmol) were added into a 10 mL microwave vial containing a magnetic stirrer bar, followed by 1,4-dioxane (1 mL) and H₂O (0.2 mL). The vessel was sealed with a cap under an argon atmosphere, and then the resulting mixture was heated to 100° C. for 2 hours under microwave. The mixture was cooled to room temperature and diluted with water (5 mL), extracted with ethyl acetate (10 mL×3), the combined organic layers were washed with brine (10 mL), dried over anhydrous sodium sulfate, concentrated in vacuo to give crude title compound. The crude title compound was purified by C-18 reversed phase HPLC column to give N-{(R)-2-[5-(2-oxo-2,3-dihydro-1H-indol-5-yl)-pyridin-3-ylamino]-2-phenyl-ethyl}-acetamide (6 mg) as a yellow solid.

### Example 31

### Preparation of 2-[5-(1-oxo-2,3-dihydro-1H-isoindol-5-yl)-pyridin-3-ylamino]-2-phenyl-acetamide

5-Bromo-2,3-dihydro-isoindol-1-one (63 mg, 0.3 mmol), bis(pinacolato)diboron (78 mg, 0.306 mmol), tris(dibenzylideneacetone)dipalladium (8.2 mg, 0.009 mmol), butyldi-1-adamantylphosphine (9.7 mg, 0.027 mmol), potassium acetate (88 mg, 0.9 mmol) were added into a 10 mL microwave vial containing a magnetic stirrer bar, followed by isopropyl acetate (0.75 mL). The vessel was sealed with a cap under an argon atmosphere, and then the resulting mixture was heated to 83° C. for 1 hour. After the reaction was completed as monitored by TLC and LC-MS, 2-(5-bromo-pyridin-3-ylamino)-2-phenyl-acetamide (73 mg, 0.24 mmol), potassium carbonate (99 mg, 0.72 mmol), DME (0.75 mL) and H₂O (0.3 mL) were added into the above mixture successively. The vessel was sealed with a cap under an argon atmosphere, and then the reaction mixture was heated to 90° C. for 40 minutes under microwave. The mixture was cooled to room temperature and diluted with water (5 mL), extracted with ethyl acetate (10 mL×3). The combined organic layers were washed with brine (10 mL), dried over anhydrous sodium sulfate, concentrated in vacuo to give crude 2-[5-(1-oxo-2,3-dihydro-1H-isoindol-5-yl)-pyridin-3-ylamino]-2-phenyl-acetamide. The crude compound was purified by C-18 reversed phase HPLC column to give 2-[5-(1-oxo-2,3-dihydro-1H-isoindol-5-yl)-pyridin-3-ylamino]-2-phenyl-acetamide (30 mg) as a white solid.

### Example 32

### Preparation of 2-[5-(3,3-dimethyl-2-oxo-2,3-dihydro-1H-indol-5-yl)-pyridin-3-ylamino]-2-phenyl-acetamide

Under an Ar atmosphere, a mixture of (5-((2-amino-2-oxo-1-phenylethyl)amino)pyridin-3-yl)boronic acid (272 mg, 1 mmol), 5-bromo-3,3-dimethyl-1,3-dihydro-indol-2-one (240 mg, 1 mmol), tetrakis(triphenylphosphine)palladium (60 mg) and potassium carbonate (280 mg, 2 mmol) in DME/H₂O (5:1, 8 mL) was heated at 90° C. under microwave for 60 mins. Then the residue was partitioned between EtOAc and brine. The aqueous layer was separated and extracted with EtOAc. The combined organic layers were concentrated and the residue was purified by Prep-HPLC to afford 2-[5-(3,3-dimethyl-2-oxo-2,3-dihydro-1H-indol-5-yl)-pyridin-3-ylamino]-2-phenyl-acetamide (77 mg).

### Example 33

### Preparation of 2-[5-(2-oxo-1,2,3,4-tetrahydro-quinolin-6-yl)-pyridin-3-ylamino]-2-phenyl-acetamide

6-Bromo-3,4-dihydro-1H-quinolin-2-one (45 mg, 0.2 mmol), bis(pinacolato)diboron (51 mg, 0.204 mmol), tris(dibenzylideneacetone)dipalladium (5.5 mg, 0.006 mmol), butyldi-1-adamantylphosphine (6.5 mg, 0.018 mmol), potassium acetate (59 mg, 0.6 mmol) were added into a 10 mL microwave vial containing a magnetic stirrer bar, followed by isopropyl acetate (0.45 mL). The vessel was sealed with a cap under an argon atmosphere, and then the resulting mixture was heated to 83° C. for 1 hour. After the reaction was completed as monitored by TLC and LC-MS, 2-(5-bromo-pyridin-3-ylamino)-2-phenyl-acetamide (61 mg, 0.2 mmol), potassium carbonate (81 mg, 0.6 mmol), isopropyl acetate (0.55 mL) and H₂O (0.2 mL) were added into the above mixture successively. The vessel was sealed with a cap under an argon atmosphere, then the reaction mixture was heated to 90° C. for 40 mins under microwave. The mixture was cooled to room temperature and diluted with water (5 mL), extracted with ethyl acetate (10 mL×3), and then the combined organic layers were washed with brine (10 mL), dried over anhydrous sodium sulfate, concentrated in vacuo to give crude 2-[5-(2-oxo-1,2,3,4-tetrahydro-quinolin-6-yl)-pyridin-3-ylamino]-2-phenyl-acetamide. The crude compound was purified by C-18 reversed phase HPLC column to give 2-[5-(2-oxo-1,2,3,4-tetrahydro-quinolin-6-yl)-pyridin-3-ylamino]-2-phenyl-acetamide (19 mg) as a white solid.

### Example 34

### Preparation of 2-[5-(7-fluoro-2-oxo-1,2,3,4-tetrahydro-quinolin-6-yl)-pyridin-3-ylamino]-2-phenyl-acetamide

6-Bromo-7-fluoro-3,4-dihydro-1H-quinolin-2-one (73 mg, 0.3 mmol), bis(pinacolato)diboron (78 mg, 0.306 mmol), tris(dibenzylideneacetone)dipalladium (8.2 mg, 0.009 mmol), butyldi-1-adamantylphosphine (9.7 mg, 0.027 mmol), potassium acetate (88 mg, 0.9 mmol) were added into a 10 mL microwave vial containing a magnetic stirrer bar, followed by isopropyl acetate (0.75 mL). The vessel was sealed with a cap under an argon atmosphere, and then the resulting mixture was heated to 83° C. for 1 hour. After the reaction was completed as monitored by TLC and LC-MS, 2-(5-bromo-pyridin-3-ylamino)-2-phenyl-acetamide (79 mg, 0.26 mmol), potassium carbonate (124 mg, 0.9 mmol), DME (0.75 mL) and H₂O (0.3 mL) were added into the above mixture successively. The vessel was sealed with a cap under an argon atmosphere, and then the reaction mixture was heated to 90° C. for 40 mins under microwave. The mixture was cooled to room temperature and diluted with water (5 mL), extracted with ethyl acetate (10 mL×3), and then the combined organic layers were washed with brine (10 mL), dried over anhydrous sodium sulfate, concentrated in vacuo to give crude 2-[5-(7-fluoro-2-oxo-1,2,3,4-tetrahydro-quinolin-6-yl)-pyridin-3-ylamino]-2-phenyl-acetamide. The crude compound was purified by C-18 reversed phase HPLC column to give 2-[5-(7-fluoro-2-oxo-1,2,3,4-tetrahydro-quinolin-6-yl)-pyridin-3-ylamino]-2-phenyl-acetamide (8 mg) as a white solid.

### Example 35

### Preparation of 2-(5-benzo[1,3]dioxol-5-yl-pyridin-3-ylamino)-2-phenyl-acetamide

2-(5-Bromo-pyridin-3-ylamino)-2-phenyl-acetamide (61 mg, 0.2 mmol), 3,4-methylenedioxphenylboronic acid (43 mg, 0.26 mmol), tetrakis(triphenylphosphine) palladium (11 mg, 0.01 mmol) and potassium carbonate (81 mg, 0.6 mmol) were added into a 10 mL microwave vial containing a magnetic stirrer bar, followed by DME (1 mL) and H₂O (0.2 mL). The vessel was sealed with a cap under an argon atmosphere, then the resulting mixture was heated to 90° C. for 40 minutes under microwave. The mixture was cooled to room temperature and diluted with water (5 mL), extracted with ethyl acetate (10 mL×3), and then the combined organic layers were washed with brine (10 mL), dried over anhydrous sodium sulfate, concentrated in vacuo to give crude 2-(5-benzo[1,3]dioxol-5-yl-pyridin-3-ylamino)-2-phenyl-acetamide (28 mg). The crude compound was purified by C-18 reversed phase HPLC column to give 2-(5-benzo[1,3]dioxol-5-yl-pyridin-3-ylamino)-2-phenyl-acetamide (28 mg) as a white solid.

### Example 36

### Preparation of 2-[5-(2,3-dihydro-benzo[1,4]dioxin-6-yl)-pyridin-3-ylamino]-2-phenyl-acetamide

2-(5-Bromo-pyridin-3-ylamino)-2-phenyl-acetamide (61 mg, 0.2 mmol), 1,4-benzodioxane-6-boronic acid (47 mg, 0.26 mmol), tetrakis(triphenylphosphine)palladium (11 mg, 0.01 mmol) and potassium carbonate (81 mg, 0.6 mmol) were added into a 10 mL microwave vial containing a magnetic stirrer bar, followed by DME (1 mL) and H₂O (0.2 mL). The vessel was sealed with a cap under an argon atmosphere, then the resulting mixture was heated to 90° C. for 40 mins under microwave. The mixture was cooled to room temperature and diluted with water (5 mL), extracted with ethyl acetate (10 mL×3), and then the combined organic layers were washed with brine (10 mL), dried over anhydrous sodium sulfate, concentrated in vacuo to give crude 2-[5-(2,3-dihydro-benzo[1,4]dioxin-6-yl)-pyridin-3-ylamino]-2-phenyl-acetamide. The crude compound was purified by C-18 reversed phase HPLC column to give 2-[5-(2,3-dihydro-benzo[1,4]dioxin-6-yl)-pyridin-3-ylamino]-2-phenyl-acetamide (29 mg) as a white solid.

### Example 37

### Preparation of 2-[5-(1H-indazol-5-yl)-pyridin-3-ylamino]-2-phenyl-acetamide

### Step 1: Preparation of indazole-5-boronic acid pinacol ester

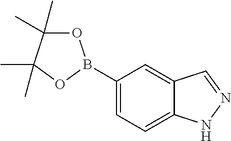 [SMILES: CC1(C)OB(c2ccc3[nH]ncc3c2)OC1(C)C]

To a solution of 5-bromoindazole (1.97 g, 10 mmol) in 1,4-dioxane (50 mL) was added bis(pinacolato)diboron (2.67 g, 10.5 mmol), 1,1-bis(diphenylphosphino)ferrocene-palladium(II) dichloride dichloromethane complex (0.82 g, 1 mmol), and potassium acetate (2.0 g, 20 mmol). The resulting mixture was degassed and then stirred overnight at 80° C. under an Ar atmosphere. After the reaction was completed as monitored by LC-MS, the mixture was diluted with water (200 mL) and then extracted with EtOAc. The combined organic layers were washed with water and brine, and then dried. The solvent was concentrated and the residue was purified by column chromatography (EtOAc/Pet=1:1) to give indazole-5-boronic acid pinacol ester (1.0 g)

### Step 2: Preparation of 2-[5-(1H-indazol-5-yl)-pyridin-3-ylamino]-2-phenyl-acetamide

To a solution of 2-(5-bromo-pyridin-3-ylamino)-2-phenyl-acetamide (306 mg, 1.0 mmol) in DME/H₂O (5:1, 12 mL) was added Pd(PPh₃)₄ (230 mg, 0.2 mmol), K₂CO₃ (276 mg, 2.0 mmol) and indazole-5-boronic acid pinacol ester (244 mg, 1.0 mmol). The resulting mixture was degassed and then stirred for 10 hours at 95° C. under an Ar atmosphere. After cooling, the mixture was diluted with water (50 mL) and then extracted with EtOAc (2×75 mL). The combined organic layers were washed with water and brine, and then dried. The solvent was concentrated and the residue was purified by Prep-HPLC to give 2-[5-(1H-indazol-5-yl)-pyridin-3-ylamino]-2-phenyl-acetamide (50 mg).

### Example 38

### Preparation of (R)-2-[5-(1H-indazol-5-yl)-pyridin-3-ylamino]-2-phenyl-acetamide

Chiral separation of the two enantiomers from 2-[5-(1H-indazol-5-yl)-pyridin-3-ylamino]-2-phenyl-acetamide (35 mg) gave chiral (R)-2-[5-(1H-indazol-5-yl)-pyridin-3-ylamino]-2-phenyl-acetamide (6 mg).

### Example 39

### Preparation of (S)-2-[5-(1H-indazol-5-yl)-pyridin-3-ylamino]-2-phenyl-acetamide

Chiral separation of the two enantiomers from 2-[5-(1H-indazol-5-yl)-pyridin-3-ylamino]-2-phenyl-acetamide (35 mg) gave chiral (S)-2-[5-(1H-indazol-5-yl)-pyridin-3-ylamino]-2-phenyl-acetamide (6 mg).

### Example 40

### Preparation of N-{(R)-2-[5-(1H-indazol-5-yl)-pyridin-3-ylamino]-2-phenyl-ethyl}-acetamide

5-Bromo-1H-indazole (60 mg, 0.3 mmol), bis(pinacolato)diboron (78 mg, 0.306 mmol), tris(dibenzylideneacetone)dipalladium (8.2 mg, 0.009 mmol), butyldi-1-adamantylphosphine (9.7 mg, 0.027 mmol), potassium acetate (88 mg, 0.9 mmol) were added into a 10 mL microwave vial containing a magnetic stirrer bar, followed by DME (0.75 mL). The vessel was sealed with a cap under an argon atmosphere, and then the resulting mixture was heated to 83° C. for 1 hour. N-[(R)-2-(5-bromo-pyridin-3-ylamino)-2-phenyl-ethyl]-acetamide (80 mg, 0.24 mmol), potassium carbonate (99 mg, 0.72 mmol), 1,4-dioxane (0.75 mL) and H₂O (0.3 mL) were added into the above mixture successively. The vessel was sealed with a cap under an argon atmosphere, and then the reaction mixture was heated to 100° C. for 2 hours under microwave. The mixture was cooled to room temperature and diluted with water (5 mL), extracted with ethyl acetate (10 mL×3), and then the combined organic layers were washed with brine (10 mL), dried over anhydrous sodium sulfate, concentrated in vacuo to give crude title compound. The crude title compound was purified by C-18 reversed phase HPLC column to give N-{(R)-2-[5-(1H-indazol-5-yl)-pyridin-3-ylamino]-2-phenyl-ethyl}-acetamide (40 mg) as a white solid.

### Example 41

### Preparation of N-{(R)-2-[5-(1H-indazol-5-yl)-pyridin-3-ylamino]-2-phenyl-ethyl}-methanesulfonamide

### Step 1: Preparation of N-[(R)-2-(5-bromo-pyridin-3-ylamino)-2-phenyl-ethyl]-methanesulfonamide

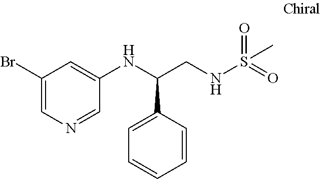 [SMILES: CS(=O)(=O)NC[C@H](Nc1cncc(Br)c1)c1ccccc1]

To a solution of crude (R)-N^{l}-(5-bromo-pyridin-3-yl)-1-phenyl-ethane-1,2-diamine (1.0 g, 3 mmol) and N,N-diisopropylethylamine (774 mg, 6 mmol) in dichloromethane (15 mL) was added methanesulfonyl chloride (342 mg, 3 mmol) dropwise at 0° C. The resulting mixture was stirred at room temperature for 2 hours. The reaction mixture was diluted with water (10 mL), and then extracted with dichloromethane (40 mL×3). The combined organic layers were washed with water (10 mL) and brine (10 mL), dried over anhydrous sodium sulfate, concentrated in vacuo to give crude N-[(R)-2-(5-bromo-pyridin-3-ylamino)-2-phenyl-ethyl]-methanesulfonamide (1.19 g) as a yellow solid which was directly used for the next step without purification.

### Step 2: Preparation of N-{(R)-2-[5-(1H-indazol-5-yl)-pyridin-3-ylamino]-2-phenyl-ethyl}-methanesulfonamide

5-Bromo-1H-indazole (60 mg, 0.3 mmol), bis(pinacolato)diboron (78 mg, 0.306 mmol), tris(dibenzylideneacetone)dipalladium (8.2 mg, 0.009 mmol), butyldi-1 adamantylphosphine (9.7 mg, 0.027 mmol), potassium acetate (88 mg, 0.9 mmol) were added into a 10 mL microwave vial containing a magnetic stirrer bar, followed by DME (0.75 mL). The vessel was sealed with a cap under an argon atmosphere, and then the resulting mixture was heated to 83° C. for 1 hour. The crude N-[(R)-2-(5-bromo-pyridin-3-ylamino)-2-phenyl-ethyl]-methanesulfonamide (89 mg, 0.24 mmol), potassium carbonate (99 mg, 0.72 mmol), 1,4-dioxane (0.75 mL) and H₂O (0.3 mL) were added into the reaction mixture successively. The vessel was sealed with a cap under an argon atmosphere, then the reaction mixture was heated to 100° C. for 2 hours under microwave. The mixture was cooled to room temperature and diluted with water (5 mL), extracted with ethyl acetate (10 mL×3), and then the combined organic layers were washed with brine (10 mL), dried over anhydrous sodium sulfate, concentrated in vacuo to give crude title compound. The crude title compound was purified by C-18 reversed phase HPLC column to give N-{(R)-2-[5-(1H-indazol-5-yl)-pyridin-3-ylamino]-2-phenyl-ethyl}-methanesulfonamide (12 mg) as a white solid.

### Example 42

### Preparation of 2-[5-(3-fluoro-1H-indazol-5-yl)-pyridin-3-ylamino]-2-phenyl-acetamide

### Step 1: Preparation of 5-bromo-3-fluoro-1H-indazole

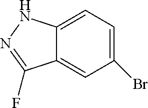 [SMILES: Fc1n[nH]c2ccc(Br)cc12]

A mixture of 5-bromo-1H-indazole (10.18 g, 51.94 mmol), 4,5-dihydropyrane (13.09 g, 155.82 mmol) and PTSA (0.98 g, 5.19 mmol) in DCM (200 mL) was stirred at room temperature and monitored by LC-MS. After all starting materials were consumed, saturated NaHCO₃ solution was added to the reaction. The mixture was extracted with DCM (200 mL×3) and the combined DCM layers were washed with brine and dried over Na₂SO₄. After concentration, the residue was purified by chromatography on silica gel column to give the crude 5-bromo-3-fluoro-1H-indazole (13.34 g). To a solution of the crude 5-bromo-3-fluoro-1H-indazole (13.34 g, 47.64 mmol) in CH₃CN (200 mL) was added Select fluor (33.73 g, 95.28 mmol) and AcOH (5 mL) successively. The mixture was heated to reflux and monitored by LC-MS. After 2 hours, starting material was fully consumed. The mixture was cooled to room temperature and diluted with water (150 mL), extracted with ethyl acetate (100 mL×3), and then the combined organic layers were washed with brine (100 mL), dried over anhydrous sodium sulfate, concentrated in vacuo to give crude compound. The crude compound purified by flash column to give 5-bromo-3-fluoro-1H-indazole (5.1 g).

### Step 2: Preparation of 3-fluoro-5-(4,4,5,5-tetramethyl-[1,3,2]dioxaborolan-2-yl)-1H-indazole

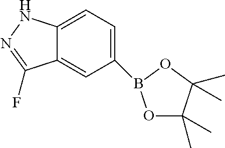 [SMILES: CC1(C)OB(c2ccc3[nH]nc(F)c3c2)OC1(C)C]

A mixture of the 5-bromo-3-fluoro-1H-indazole (2.0 g, 9.44 mmol), bis(pinacolato)diboron (2.4 g, 9.44 mmol), KOAc (1.86 g, 18.88 mmol) and Pd(dppf)Cl₂ dichloromethane complex (767 mg, 0.94 mmol) in dioxane (50 mL) was heated to reflux with stirring. After the starting material was consumed, the reaction was concentrated. The residue was purified by flash column to give 3-fluoro-5-(4,4,5,5-tetramethyl-[1,3,2]dioxaborolan-2-yl)-1H-indazole (2.0 g) as a white solid.

### Step 3: Preparation of 2-[5-(3-fluoro-1H-indazol-5-yl)-pyridin-3-ylamino]-2-phenyl-acetamide

3-Fluoro-5-(4,4,5,5-tetramethyl-[1,3,2]dioxaborolan-2-yl)-1H-indazole (120 mg, 0.46 mol), 2-(5-bromo-pyridin-3-ylamino)-2-phenyl-acetamide (146 mg, 0.48 mmol) and K₂CO₃ (127 mg, 0.92 mmol) were mixed in dimethoxyethane and H₂O mixture (2.5 mL/0.5 mL). The mixture was degassed and charged with N₂. Then Pd(PPh₃)₄ (26 mg, 0.02 mmol) was added. The mixture was heated to reflux and stirred overnight. The mixture was cooled to room temperature and diluted with water (5 mL), extracted with ethyl acetate (10 mL×3), and then the combined organic layers were washed with brine (10 mL), dried over anhydrous sodium sulfate, concentrated in vacuo to give crude compound. The crude product was purified by Prep-HPLC to give 2-[5-(3-fluoro-1H-indazol-5-yl)-pyridin-3-ylamino]-2-phenyl-acetamide (10.9 mg).

### Example 43

### Preparation of N-{(R)-2-[5-(3-fluoro-1H-indazol-5-yl)-pyridin-3-ylamino]-2-phenyl-ethyl}-propionamide

### Step 1: Preparation of N-[(R)-2-(5-bromo-pyridin-3-ylamino)-2-phenyl-ethyl]-propionamide

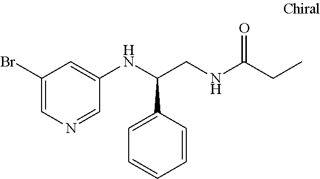 [SMILES: CCC(=O)NC[C@H](Nc1cncc(Br)c1)c1ccccc1]

To a solution of crude (R)-N^{l}-(5-bromo-pyridin-3-yl)-1-phenyl-ethane-1,2-diamine (150 mg, 0.5 mmol) and N,N-diisopropylethylamine (129 mg, 1 mmol) in dichloromethane (3 mL) was added propionyl chloride (47 mg, 0.5 mmol) dropwise at 0° C. The resulting mixture was stirred at room temperature overnight. The reaction mixture was diluted with water (5 mL), and then extracted with dichloromethane (20 mL×3). The combined organic layers were washed with water (5 mL) and brine (5 mL), dried over anhydrous sodium sulfate, concentrated in vacuo to give N-[(R)-2-(5-bromo-pyridin-3-ylamino)-2-phenyl-ethyl]-propionamide (210 mg) as yellow oil which was directly used for the next step without purification.

### Step 2: Preparation of N-{(R)-2-[5-(3-fluoro-1H-indazol-5-yl)-pyridin-3-ylamino]-2-phenyl-ethyl}-propionamide

5-Bromo-3-fluoro-1H-indazole (107 mg, 0.5 mmol), bis(pinacolato)diboron (130 mg, 0.51 mmol), tris(dibenzylideneacetone)dipalladium (14 mg, 0.015 mmol), butyldi-1-adamantylphosphine (16 mg, 0.045 mmol), potassium acetate (147 mg, 1.5 mmol) were added into a 10 mL microwave vial containing a magnetic stirrer bar, followed by DME (0.75 mL). The vessel was sealed with a cap under an argon atmosphere, then the resulting mixture was heated to 83° C. for 1 hour. Then N-[(R)-2-(5-bromo-pyridin-3-ylamino)-2-phenyl-ethyl]-propionamide (210 mg, crude), potassium carbonate (99 mg, 0.72 mmol), 1,4-dioxane (0.75 mL) and H₂O (0.3 mL) were added into the reaction mixture successively. The vessel was sealed with a cap under an argon atmosphere, and then the reaction mixture was heated to 100° C. for 2 hours under microwave. The mixture was cooled to room temperature and diluted with water (5 mL), extracted with ethyl acetate (10 mL×3), and then the combined organic layers were washed with brine (10 mL), dried over anhydrous sodium sulfate, concentrated in vacuo to give crude title compound. The crude title compound was purified by C-18 reversed phase HPLC column to give N-{(R)-2-[5-(3-fluoro-1H-indazol-5-yl)-pyridin-3-ylamino]-2-phenyl-ethyl}-propionamide (45 mg) as a yellow solid.

### Example 44

### Preparation of N-{(R)-2-[5-(3-fluoro-1H-indazol-5-yl)-pyridin-3-ylamino]-2-phenyl-ethyl}-isobutyramide

### Step 1: Preparation of N-[(R)-2-(5-bromo-pyridin-3-ylamino)-2-phenyl-ethyl]-isobutyramide

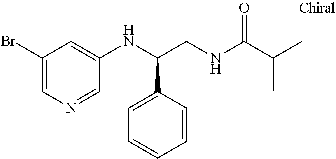 [SMILES: CC(C)C(=O)NC[C@H](Nc1cncc(Br)c1)c1ccccc1]

To a solution of crude (R)-N^{l}-(5 -bromo-pyridin-3-yl)-1-phenyl-ethane-1,2-diamine (150 mg, 0.5 mmol) and N,N-diisopropylethylamine (129 mg, 1 mmol) in dichloromethane (3 mL) was added 2-methylpropanoyl chloride (54 mg, 0.5 mmol) dropwise at 0° C. The resulting mixture was stirred at room temperature overnight. The reaction mixture was diluted with water (5 mL), and then extracted with dichloromethane (20 mL×3). The combined organic layers were washed with water (5 mL) and brine (5 mL), dried over anhydrous sodium sulfate, concentrated in vacuo to give N-[(R)-2-(5-bromo-pyridin-3-ylamino)-2-phenyl-ethyl]-isobutyramide (220 mg) as yellow oil which was directly used for the next step without purification.

### Step 2: Preparation of N-{(R)-2-[5-(3-fluoro-1H-indazol-5-yl)-pyridin-3-ylamino]-2-phenyl-ethyl}-isobutyramide

5-Bromo-3-fluoro-1H-indazole (107 mg, 0.5 mmol), bis(pinacolato)diboron (130 mg, 0.51 mmol), tris(dibenzylideneacetone)dipalladium (14 mg, 0.015 mmol), butyldi-1-adamantylphosphine (16 mg, 0.045 mmol), potassium acetate (147 mg, 1.5 mmol) were added into a 10 mL microwave vial containing a magnetic stirrer bar, followed by DME (0.75 mL). The vessel was sealed with a cap under an argon atmosphere, and then the resulting mixture was heated to 83° C. for 1 hour. Then N-[(R)-2-(5-bromo-pyridin-3-ylamino)-2-phenyl-ethyl]-isobutyramide (220 mg, crude), potassium carbonate (99 mg, 0.72 mmol), 1,4-dioxane (0.75 mL) and H₂O (0.3 mL) were added into the reaction mixture successively. The vessel was sealed with a cap under an argon atmosphere, and then the reaction mixture was heated to 100° C. for 2 hours under microwave. The mixture was cooled to room temperature and diluted with water (5 mL), extracted with ethyl acetate (10 mL×3), and then the combined organic layers were washed with brine (10 mL), dried over anhydrous sodium sulfate, concentrated in vacuo to give crude title compound. The crude title compound was purified by C-18 reversed phase HPLC column to give N-{(R)-2-[5-(3-fluoro-1H-indazol-5-yl)-pyridin-3-ylamino]-2-phenyl-ethyl}-isobutyramide (63 mg) as a yellow solid.

### Example 45

### Preparation of (R)-N^{l}-[5-(3-fluoro-1H-indazol-5-yl)-pyridin-3-yl]-N²-(2-methoxy-ethyl)-1-phenyl-ethane-1,2-diamine

### Step 1: Preparation of N-[(R)-2-(5-bromo-pyridin-3-ylamino)-2-phenyl-ethyl]-2-methoxy-acetamide

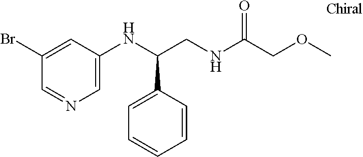 [SMILES: COCC(=O)NC[C@H](Nc1cncc(Br)c1)c1ccccc1]

(R)-N^{l}-5-Bromo-pyridin-3-yl)-1-phenyl-ethane-1,2-diamine (150 mg, 0.51 mmol), methoxy-acetic acid (70 mg, 0.77 mmol), HATU (387 mg, 1.01 mmol) and Et₃N (0.5 mL) were mixed in DMF (5 mL) and the mixture was stirred at room temperature for 3 hours. Then H₂O (20 mL) was added and the mixture was extracted with ethyl acetate (30 mL×3). The combined organic layers were dried and concentrated. The residue was purified by flash column to give 110 mg of N-[(R)-2-(5 -bromo-pyridin-3-ylamino)-2-phenyl-ethyl]-2-methoxy-acetamide.

### Step 2: Preparation of N-{(R)-2-[5-(3-fluoro-1H-indazol-5-yl)-pyridin-3-ylamino]-2-phenyl-ethyl}-2-methoxy-acetamide

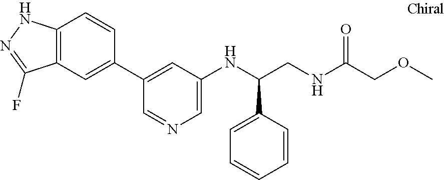 [SMILES: COCC(=O)NC[C@H](Nc1cncc(-c2ccc3[nH]nc(F)c3c2)c1)c1ccccc1]

A mixture of N-[(R)-2-(5 -bromo-pyridin-3-ylamino)-2-phenyl-ethyl]-2-methoxy-acetamide (115 mg, 0.44 mmol), 3-fluoro-5-(4,4,5,5-tetramethyl-[1,3,2]dioxaborolan-2-yl)-1H-indazole (160 mg, 0.44 mmol) and K₂CO₃ (120 mg, 0.88 mmol) in dioxane/H₂O (5 mL/1 mL) was degassed and charged with N₂. Then Pd(PPh₃)₄ was added and the reaction mixture was heated to reflux with stirring. After all the starting material was consumed, the reaction was concentrated under reduced pressure and the residue was purified by flash column to give N-{(R)-2-[5-(3-fluoro-1H-indazol-5-yl)-pyridin-3-ylamino]-2-phenyl-ethyl}-2-methoxy-acetamide (80 mg).

### Step 3: Preparation of (R)-N^{l}-[5-(3-fluoro-1H-indazol-5-yl)-pyridin-3-yl]-N²-(2-methoxy-ethyl)-1-phenyl-ethane-1,2-diamine

To a solution of N-{(R)-2-[5-(3-fluoro-1H-indazol-5-yl)-pyridin-3-ylamino]-2-phenyl-ethyl}-2-methoxy-acetamide (80 mg, 0.19 mmol) in THF (5 mL) was added BH₃ solution (3.8 mL, 3.8 mmol). The mixture was heated to 80° C. and stirred overnight. The reaction was cooled down to room temperature and carefully quenched with 1M HCl. The resulting mixture was concentrated under reduced pressure to half of its original volume. Then it was neutralized with saturated NaHCO₃ and extracted with ethyl acetate (30 mL×3). The organic layer was dried and concentrated, and then the residue was purified by Prep-HPLC to give (R)-N^{l}-[5-(3-fluoro-1H-indazol-5-yl)-pyridin-3-yl]-N²-(2-methoxy-ethyl)-1-phenyl-ethane-1,2-diamine (30 mg).

### Example 46

### Preparation of 2-hydroxy-N-{(R)-2-[5-(3-methyl-1H-indazol-5-yl)-pyridin-3-ylamino]-2-phenyl-ethyl}-acetamide

### Step 1: Preparation of N-[(R)-2-(5-bromo-pyridin-3-ylamino)-2-phenyl-ethyl]-2-hydroxy-acetamide

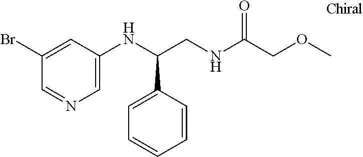 [SMILES: COCC(=O)NC[C@H](Nc1cncc(Br)c1)c1ccccc1]

A mixture of (R)-N^{l}-(5-bromo-pyridin-3-yl)-1-phenyl-ethane-1,2-diamine (200 mg, 0.69 mmol), glycolic acid (79 mg, 1.03 mmol), HATU (525 mg, 1.38 mmol) and TEA (2 mL) in DMF (5 mL) was stirred for 3 hours. Then H₂O (20 mL) was added and the aqueous layer was extracted with ethyl acetate (30 mL×3). The combined organic layers were dried and concentrated. The residue was purified by flash column to give 160 mg of N-[(R)-2-(5-bromo-pyridin-3-ylamino)-2-phenyl-ethyl]-2-hydroxy-acetamide.

### Step 2: Preparation of N-{(R)-2-[5-(3-fluoro-1H-indazol-5-yl)-pyridin-3-ylamino]-2-phenyl-ethyl}-2-hydroxy-acetamide

A mixture of N-[(R)-2-(5-Bromo-pyridin-3-ylamino)-2-phenyl-ethyl]-2-hydroxy-acetamide (400 mg, 1.15 mmol), 3-fluoro-5-(4,4,5,5-tetramethyl-[1,3,2]dioxaborolan-2-yl)-1H-indazole (360 mg, 1.38 mmol) and K₂CO₃ (635 mg, 4.60 mmol) in dioxane/H₂O (5 mL/1 mL) was degassed and charged with N₂. Then Pd(PPh₃)₄ (69 mg, 0.06 mmol) was added and the mixture was heated to 150° C. in microwave reactor for 2 hours. The reaction mixture was purified by flash column to give 200 mg of N-{(R)-2-[5-(3-fluoro-1H-indazol-5-yl)-pyridin-3-ylamino]-2-phenyl-ethyl}-2-hydroxy-acetamide.

### Example 47

### Preparation of 2-{(R)-2-[5-(3-fluoro-1H-indazol-5-yl)-pyridin-3-ylamino]-2-phenyl-ethylamino}-ethanol

N-{(R)-2-[5-(3-fluoro-1H-indazol-5-yl)-pyridin-3-ylamino]-2-phenyl-ethyl}-2-hydroxy-acetamide (150 mg, 0.37 mmol) was dissolved in THF (5 mL) and a solution of BH₃ (7.5 mL, 1.0 M in THF) was added. The mixture was heated to 80° C. and stirred overnight. After cooling down to room temperature, the reaction mixture was quenched with 1M HCl, and then concentrated under reduced pressure to remove half of the solvent. Then saturated NaHCO₃ was added to the residue to neutralization. The mixture was extracted with ethyl acetate (30 mL×3). The combined organic layers were dried and concentrated. The residue was purified by Prep-HPLC and 30 mg of 2-{(R)-2-[5-(3-fluoro-1H-indazol-5-yl)-pyridin-3-ylamino]-2-phenyl-ethylamino}-ethanol was obtained.

### Example 48

### Preparation of (R)-2-[5-(3-methyl-1H-indazol-5-yl)-pyridin-3-ylamino]-2-phenyl-acetamide

A mixture of 3-methyl-5-(4,4,5,5-tetramethyl-[1,3,2]dioxaborolan-2-yl)-1H-indazole (590 mg, 2.29 mmol), 2-(5-bromo-pyridin-3-ylamino)-2-phenyl-acetamide (700 mg, 2.29 mmol) and K₂CO₃ (630 mg, 4.58 mmol) in dioxane/H₂O(10 mL/1 mL) was degassed and charged with N₂. Then Pd(PPh₃)₄ (265 mg, 0.23 mmol) was added and the reaction mixture was heated to reflux overnight with stirring. The solvent was removed and then the residue was purified by flash column to give 200 mg of 2-[5-(3-methyl-1H-indazol-5-yl)-pyridin-3-ylamino]-2-phenyl-acetamide. Chiral separation of 2-[5-(3-methyl-1H-indazol-5-yl)-pyridin-3-ylamino]-2-phenyl-acetamide afforded (R)-2-[5-(3-methyl-1H-indazol-5-yl)-pyridin-3-ylamino]-2-phenyl-acetamide.

### Example 49

### Preparation of (S)-2-[5-(3-methyl-1H-indazol-5-yl)-pyridin-3-ylamino]-2-phenyl-acetamide

Chiral separation of 200 mg of 2-[5-(3-methyl-1H-indazol-5-yl)-pyridin-3-ylamino]-2-phenyl-acetamide afforded (S)-2-[5-(3-methyl-1H-indazol-5-yl)-pyridin-3-ylamino]-2-phenyl-acetamide.

### Example 50

### Preparation of N-{2-[5-(3-methyl-1H-indazol-5-yl)-pyridin-3-ylamino]-2-phenyl-ethyl}-methanesulfonami

### Step 1: Preparation of 2-(5-bromo-pyridin-3-ylamino)-2-phenyl-ethanol

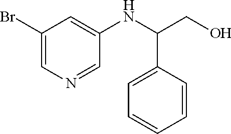 [SMILES: OCC(Nc1cncc(Br)c1)c1ccccc1]

To a solution of 3,5-dibromopyridine (735 mg, 3.1 mmol) and 2-amino-2-phenyl-ethanol (650 g, 4.7 mmol) in DMSO (8 mL) was added copper(I) iodide (59 mg, 0.31 mmol), L-proline (71 mg, 0.62 mmol) and potassium carbonate (856 mg, 6.2 mmol) successively, the resulting mixture was stirred at 100° C. overnight under argon atmosphere. The reaction mixture was cooled to room temperature and diluted with water (30 mL), then extracted with ethyl acetate (30 mL×3). The combined organic layers were washed with water (20 mL) and brine (20 mL), dried over anhydrous sodium sulfate, concentrated in vacuo to give crude 2-(5-bromo-pyridin-3-ylamino)-2-phenyl-ethanol. The crude compound was purified by flash column to afford 2-(5-bromo-pyridin-3-ylamino)-2-phenyl-ethanol (2.70 mg) as yellow oil.

### Step 2: Preparation of 2-[2-(5-brorno-pyridin-3-ylarnino)-2-phenyl-ethyl]-isoindole-1,3-dione

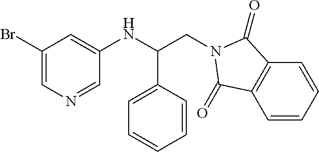 [SMILES: O=C1c2ccccc2C(=O)N1CC(Nc1cncc(Br)c1)c1ccccc1]

To a solution of 2-(5-bromo-pyridin-3-ylamino)-2-phenyl-ethanol (270 mg, 0.9 mmol), phthalimide (304 mg, 2.07 mmol) and triphenylphosphine (542 mg, 2.07 mmol) in THF (3 mL) was added diethyl azodicarboxylate (360 mg, 2.07 mmol) dropwise at 0° C. under argon atmosphere. The resulting mixture was stirred at 0° C. at room temperature overnight. The reaction mixture was diluted with water (10 mL), then extracted with ethyl acetate (30 mL×3). The combined organic layers were washed with water (10 mL) and brine (10 mL), then dried over anhydrous sodium sulfate, concentrated in vacuo to give crude 2-[2-(5-bromo-pyridin-3-ylamino)-2-phenyl-ethyl]-isoindole-1,3-dione. The crude compound was purified by flash column to afford 2-[2-(5-bromo-pyridin-3-ylamino)-2-phenyl-ethyl]-isoindole-1,3-dione (300 mg) as yellow oil.

### Step 3: Preparation of N^{l}-(5-bromo-pyridin-3-yl)-1-phenyl-ethane-1,2-diamine

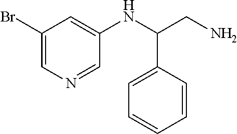 [SMILES: NCC(Nc1cncc(Br)c1)c1ccccc1]

To a solution of crude 2-[2-(5-bromo-pyridin-3-ylamino)-2-phenyl-ethyl]-isoindole-1,3-dione (300 g) in ethanol (3 mL) was added hydrazine hydrate (3 mL), the resulting mixture was stirred at 80° C. for 1 hour. After the reaction was completed as monitored by TLC and LC-MS, the reaction mixture was cooled to room temperature and diluted with water (10 mL), then extracted with ethyl acetate (30 mL×3). The combined organic layers were washed with water (10 mL) and brine (10 mL), then dried over anhydrous sodium sulfate, concentrated in vacuo to give crude N^{l}-5-bromo-pyridin-3-yl)-1-phenyl-ethane-1,2-diamine (170 mg) as yellow oil which was directly used for the next step without purification.

### Step 4: Preparation of N-[2-(5-bromo-pyridin-3-ylamino)-2-phenyl-ethyl]-methanesulfonamide

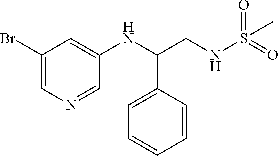 [SMILES: CS(=O)(=O)NCC(Nc1cncc(Br)c1)c1ccccc1]

To a solution of crude N^{l}-(5-bromo-pyridin-3-yl)-1-phenyl-ethane-1,2-diamine (170 mg, 0.58 mmol) and N,N-diisopropylethylamine (150 mg, 1.16 mmol) in dichloromethane (3 mL) was added methanesulfonyl chloride (66 mg, 0.58 mmol) dropwise at 0° C. The resulting mixture was stirred at 0° C. at room temperature for 1 hour. The reaction mixture was diluted with water (10 mL), then extracted with dichloromethane (30 mL×3). The combined organic layers were washed with water (10 mL) and brine (10 mL), then dried over anhydrous sodium sulfate, concentrated in vacuo to give crude N-[2-(5-bromo-pyridin-3-ylamino)-2-phenyl-ethyl]-methanesulfonamide (200 mg) as a white solid.

### Step 5: Preparation of N-{2-[5-(3-methyl-1H-indazol-5-yl)-pyridin-3-ylamino]-2-phenyl-ethyl}-methanesulfonamide

5-Bromo-3-methyl-1H-indazole (101 mg, 0.48 mmol), bis(pinacolato)diboron (125 mg, 0.49 mmol), tris(dibenzylideneacetone)dipalladium (13 mg, 0.014 mmol), butyldi-1-adamantylphosphine (16 mg, 0.043 mmol), potassium acetate (141 mg, 1.44 mmol) were added into a 10 mL microwave vial containing a magnetic stirrer bar, followed by DME (1 mL). The vessel was sealed with a cap under an argon atmosphere, then the resulting mixture was heated to 83° C. for 1 hour. Then the crude N-[2-(5-bromo-pyridin-3-ylamino)-2-phenyl-ethyl]-methanesulfonamide (200 mg, 0.48 mmol), potassium carbonate (199 mg, 1.44 mmol), 1,4-dioxane (1 mL) and H₂O (0.4 mL) were added into the reaction mixture successively. The vessel was sealed with a cap under an argon atmosphere, the reaction mixture was heated to 100° C. for 2 hours under microwave. The mixture was cooled to room temperature and diluted with water (5 mL), extracted with ethyl acetate (10 mL×3). The combined organic layers were washed with brine (10 mL), dried over anhydrous sodium sulfate, concentrated in vacuo to give crude title compound. The crude title compound was purified by C-18 reversed phase HPLC column to give N-{2-[5-(3-methyl-1H-indazol-5-yl)-pyridin-3-ylamino]-2-phenyl-ethyl}-methanesulfonamide (67 mg) as a yellow solid.

### Example 51

### Preparation of cyclopropanesulfonic acid {(R)-2-[5-(3-methyl-1H-indazol-5-yl)-pyridin-3-ylamino]-2-phenyl-ethyl}-amide

### Step 1: Preparation of cyclopropanesulfonic acid [(R)-2-(5-bromo-pyridin-3-ylamino)-2-phenyl-ethyl]-amide

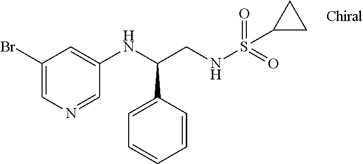 [SMILES: O=S(=O)(NC[C@H](Nc1cncc(Br)c1)c1ccccc1)C1CC1]

To a solution of crude (R)-N^{l}-(5-bromo-pyridin-3-yl)-1-phenyl-ethane-1,2-diamine (113 mg, 0.33 mmol) and N,N-diisopropylethylamine (85 mg, 0.66 mmol) in dichloromethane (2 mL) was added cyclopropanesulfonyl chloride (46 mg, 0.33 mmol) dropwise at 0° C. The resulting mixture was stirred at room temperature for 2 hours. The reaction mixture was diluted with water (5 mL), and then extracted with dichloromethane (20 mL×3). The combined organic layers were washed with water (5 mL) and brine (5 mL), then dried over anhydrous sodium sulfate, concentrated in vacuo to give cyclopropanesulfonic acid [(R)-2-(5-bromo-pyridin-3-ylamino)-2-phenyl-ethyl]-amide (150 mg) as yellow oil which was directly used for the next step without purification.

### Step 2: Preparation of cyclopropanesulfonic acid {(R)-2-[5-(3-methyl-1H-indazol-5-yl)-pyridin-3-ylamino]-2-phenyl-ethyl}-amide

5-Bromo-3-methyl-1H-indazole (63 mg, 0.3 mmol), bis(pinacolato)diboron (78 mg, 0.306 mmol), tris(dibenzylideneacetone)dipalladium (8.2 mg, 0.009 mmol), butyldi-1-adamantylphosphine (9.7 mg, 0.027 mmol), potassium acetate (88 mg, 0.9 mmol) were added into a 10 mL microwave vial containing a magnetic stirrer bar, followed by DME (1 mL). The vessel was sealed with a cap under an argon atmosphere, and then the resulting mixture was heated to 83° C. for 1 hour. Cyclopropanesulfonic acid [(R)-2-(5-bromo-pyridin-3-ylamino)-2-phenyl-ethyl]-amide (150 mg, crude), potassium carbonate (124 mg, 0.9 mmol), 1,4-dioxane (2 mL) and H₂O (0.6 mL) were added into the reaction mixture successively. The vessel was sealed with a cap under an argon atmosphere, and then the reaction mixture was heated to 100° C. for 2 hours under microwave. The mixture was cooled to room temperature and diluted with water (5 mL), extracted with ethyl acetate (10 mL×3). The combined organic layers were washed with brine (10 mL), dried over anhydrous sodium sulfate, concentrated in vacuo to give crude title compound. The crude title compound was purified by C-18 reversed phase HPLC column to give cyclopropanesulfonic acid {(R)-2-[5-(3-methyl-1H-indazol-5-yl)-pyridin-3-ylamino]-2-phenyl-ethyl}-amide (15 mg) as a white solid.

### Example 52

### Preparation of N-{(R)-2-[5-(3-methyl-1H-indazol-5-yl)-pyridin-3-ylamino]-2-phenyl-ethyl}-methanesulfonamide

### Step 1: Preparation of N-[(R)-2-(5-bromo-pyridin-3-ylamino)-2-phenyl-ethyl]-methanesulfonamide

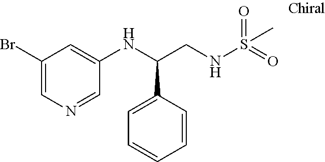 [SMILES: CS(=O)(=O)NC[C@H](Nc1cncc(Br)c1)c1ccccc1]

To a solution of crude (R)-N^{l}-(5-bromo-pyridin-3-yl)-1-phenyl-ethane-1,2-diamine (1.0 g, 3 mmol) and N,N-diisopropylethylamine (774 mg, 6 mmol) in dichloromethane (15 mL) was added methanesulfonyl chloride (342 mg, 3 mmol) dropwise at 0° C. The resulting mixture was stirred at room temperature for 2 hours. The reaction mixture was diluted with water (10 mL), and then extracted with dichloromethane (40 mL×3). The combined organic layers were washed with water (10 mL) and brine (10 mL), then dried over anhydrous sodium sulfate, concentrated in vacuo to give crude N-[(R)-2-(5-bromo-pyridin-3-ylamino)-2-phenyl-ethyl]-methanesulfonamide (1.19 g) as a yellow solid.

### Step 2: Preparation of N-{(R)-2-[5-(3-methyl-1H-indazol-5-yl)-pyridin-3-ylamino]-2-phenyl-ethyl}-methanesulfonamide

5-Bromo-3-methyl-1H-indazole (105 mg, 0.5 mmol), bis(pinacolato)diboron (130 mg, 0.51 mmol), tris(dibenzylideneacetone)dipalladium (14 mg, 0.015 mmol), butyldi-1-adamantylphosphine (16 mg, 0.045 mmol), potassium acetate (147 mg, 1.5 mmol) were added into a 10 mL microwave vial containing a magnetic stirrer bar, followed by DME (1 mL). The vessel was sealed with a cap under an argon atmosphere, and then the resulting mixture was heated to 83° C. for 1 hour. Then the crude N-[(R)-2-(5-bromo-pyridin-3-ylamino)-2-phenyl-ethyl]-methanesulfonamide (190 mg, 0.5 mmol), potassium carbonate (156 mg, 1.2 mmol), 1,4-dioxane (1 mL) and H₂O (0.4 mL) were added into the reaction mixture successively. The vessel was sealed with a cap under an argon atmosphere, and then the reaction mixture was heated to 100° C. for 2 hours under microwave. The mixture was cooled to room temperature and diluted with water (5 mL), extracted with ethyl acetate (10 mL×3). The combined organic layers were washed with brine (10 mL), dried over anhydrous sodium sulfate, concentrated in vacuo to give crude title compound. The crude title compound was purified by C-18 reversed phase HPLC column to give N-{(R)-2-[5-(3-methyl-1H-indazol-5-yl)-pyridin-3-ylamino]-2-phenyl-ethyl}-methanesulfonamide (45 mg) as a white solid.

### Example 53

### Preparation of 2-methoxy-ethanesulfonic acid {(R)-2-[5-(3-methyl-1H-indazol-5-yl)-pyridin-3-ylamino]-2-phenyl-ethyl}-amide

### Step 1: Preparation of 2-methoxy-ethanesulfonic acid [(R)-2-(5-bromo-pyridin-3-ylamino)-2-phenyl-ethyl]-amide

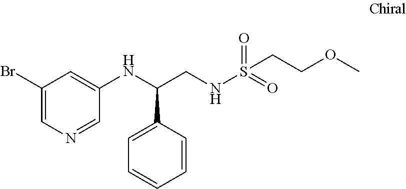 [SMILES: COCCS(=O)(=O)NC[C@H](Nc1cncc(Br)c1)c1ccccc1]

To a solution of crude (R)-N^{l}-(5-bromo-pyridin-3-yl)-1-phenyl-ethane-1,2-diamine (190 mg, 0.5 mmol) and N,N-diisopropylethylamine (129 mg, 1 mmol) in dichloromethane (2 mL) was added 2-methoxy-ethanesulfonyl chloride (79 mg, 0.5 mmol) dropwise at 0° C. The resulting mixture was stirred at room temperature overnight. The reaction mixture was diluted with water (5 mL), and then extracted with dichloromethane (20 mL×3). The combined organic layers were washed with water (5 mL) and brine (5 mL), then dried over anhydrous sodium sulfate, concentrated in vacuo to give 2-methoxy-ethanesulfonic acid [(R)-2-(5-bromo-pyridin-3-ylamino)-2-phenyl-ethyl]-amide (166 mg) as yellow oil.

### Step 2: Preparation of 2-methoxy-ethanesulfonic acid {(R)-2-[5-(3-methyl-1H-indazol-5-yl)-pyridin-3-ylamino]-2-phenyl-ethyl}-amide

5-Bromo-3-methyl-1H-indazole (105 mg, 0.5 mmol), bis(pinacolato)diboron (130 mg, 0.51 mmol), tris(dibenzylideneacetone)dipalladium (14 mg, 0.015 mmol), butyldi-1-adamantylphosphine (16 mg, 0.045 mmol), potassium acetate (147 mg, 1.5 mmol) were added into a 10 mL microwave vial containing a magnetic stirrer bar, followed by DME (1 mL). The vessel was sealed with a cap under an argon atmosphere, and then the resulting mixture was heated to 83° C. for 1 hour. Then 2-methoxy-ethanesulfonic acid [(R)-2-(5-bromo-pyridin-3-ylamino)-2-phenyl-ethyl]-amide (166 mg, crude), potassium carbonate (156 mg, 1.2 mmol), 1,4-dioxane (1 mL) and H₂O (0.4 mL) were added into the reaction mixture successively. The vessel was sealed with a cap under an argon atmosphere, and then the reaction mixture was heated to 100° C. for 2 hours under microwave. The mixture was cooled to room temperature and diluted with water (5 mL), extracted with ethyl acetate (10 mL×3). The combined organic layers were washed with brine (10 mL), dried over anhydrous sodium sulfate, concentrated in vacuo to give crude title compound. The crude title compound was purified by C-18 reversed phase HPLC column to give 2-methoxy-ethanesulfonic acid {(R)-2-[5-(3-methyl-1H-indazol-5-yl)-pyridin-3-ylamino]-2-phenyl-ethyl}-amide (40 mg) as a yellow solid.

### Example 54

### Preparation of 2-hydroxy-N-{(R)-2-[5-(3-methyl-1H-indazol-5-yl)-pyridin-3-ylamino]-2-phenyl-ethyl}-acetamide

N-[(R)-2-(5-Bromo-pyridin-3-ylamino)-2-phenyl-ethyl]-2-hydroxy-acetamide (350 mg, 1.0 mmol), 3-methyl-5-(4,4,5,5-tetramethyl-[1,3,2]dioxaborolan-2-yl)-1H-indazole (390 mg, 1.5 mmol) and K₂CO₃ (550 mg, 4.0 mmol) in dioxane/H₂O (5 mL/1 mL) was degassed and charged with N₂. Then Pd(PPh₃)₄ (58 mg, 0.05 mmol) was added and the mixture was heated to 150° C. in microwave reactor for 2 hours. The reaction mixture was purified by flash column to give 2-hydroxy-N-{(R)-2-[5-(3-methyl-1H-indazol-5-yl)-pyridin-3-ylamino]-2-phenyl-ethyl}-acetamide (120 mg).

### Example 55

### Preparation of (R)-N²-(2-methoxy-ethyl)-N^{l}-[5-(3-methyl-1H-indazol-5-yl)-pyridin-3-yl]-1-phenyl-ethane-1,2-diamine

### Step 1: Preparation of 2-methoxy-N-{(R)-2-[5-(3-methyl-1H-indazol-5-yl)-pyridin-3-ylamino]-2-phenyl-ethyl}-acetamide

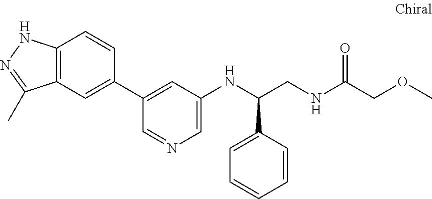 [SMILES: COCC(=O)NC[C@H](Nc1cncc(-c2ccc3[nH]nc(C)c3c2)c1)c1ccccc1]

N-[(R)-2-(5-Bromo-pyridin-3-ylamino)-2-phenyl-ethyl]-2-methoxy-acetamide (230 mg, 0.63 mmol), 3-methyl-5-(4,4,5,5-tetramethyl-[1,3,2]dioxaborolan-2-yl)-1H-indazole (196 mg, 0.76 mmol) and K₂CO₃ (210 mg, 1.52 mmol) were mixed in dioxane/H₂O (5 mL/1 mL). The mixture was degassed and charged with N₂. Then Pd(Ph₃)₄ (35 mg, 0.03 mmol) was added and the reaction mixture was heated to 150° C. in microwave reactor for 2 hours. The solvent were removed and the residue was purified by flash column (DCM/MeOH=80:1 to 20:1) to give 2-methoxy-N-{(R)-2-[5-(3-methyl-1H-indazol-5-yl)-pyridin-3-ylamino]-2-phenyl-ethyl}-acetamide (160 mg).

### Step 2: Preparation of (R)-N²-(2-methoxy-ethyl)-N^{l}-[5-(3-methyl-1H-indazol-5-yl)-pyridin-3-yl]-1-phenyl-ethane-1,2-diamine

2-Methoxy-N-{(R)-2-[5-(3-methyl-1H-indazol-5-yl)-pyridin-3-ylamino]-2-phenyl-ethyl}-acetamide (160 mg, 0.39 mmol) was dissolved in THF (5 mL) and a solution of BH₃ (7.8 mL, 1.0 M in THF) was added. The reaction was heated to 80° C. and stirred overnight. After cooling down to room temperature, the reaction mixture was quenched with 1M HCl. The mixture was concentrated to half of its volume. Then saturated NaHCO₃ solution was added and the mixture was extracted with ethyl acetate (30 mL×3). The combined organic layers were dried and concentrated. The residue was purified by Prep-HPLC to afford (R)-N²-(2-methoxy-ethyl)-N^{l}-[5-(3-methyl-1H-indazol-5-yl)-pyridin-3-yl]-1-phenyl-ethane-1,2-diamine (30 mg).

### Example 56

### Preparation of 2-{(R)-2-[5-(3-methyl-1H-indazol-5-yl)-pyridin-3-ylamino]-2-phenyl-ethylamino}-ethanol

N-{(R)-2-[5-(3-fluoro-1H-indazol-5-yl)-pyridin-3-ylamino]-2-phenyl-ethyl}-2-hydroxy-acetamide (100 mg, 0.25 mmol) was dissolved in THF (5 mL) and a solution of BH₃ (5.0 mL, 1.0 M in THF) was added. The mixture was heated to 80° C. and stirred overnight. After cooling down to room temperature, the reaction mixture was quenched with 1M HCl. The reaction mixture was concentrated under reduced pressure to remove half of the solvent. Then saturated NaHCO₃ was added to the residue to neutralization and the mixture was extracted with ethyl acetate (30 mL×3). The combined organic layers were dried and concentrated. The residue was purified by Prep-HPLC to afford 20 mg of 2-{(R)-2-[5-(3-methyl-1H-indazol-5-yl)-pyridin-3-ylamino]-2-phenyl-ethylamino}-ethanol.

### Example 57

### Preparation of cyclohexanecarboxylic acid {(R)-2-[5-(3-methyl-1H-indazol-5-yl)-pyridin-3-ylamino]-2-phenyl-ethyl}-amide

### Step 1: Preparation of cyclohexanecarboxylic acid [(R)-2-(5-bromo-pyridin-3-ylamino)-2-phenyl-ethyl]-amide

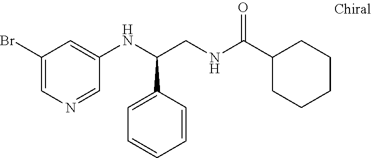 [SMILES: O=C(NC[C@H](Nc1cncc(Br)c1)c1ccccc1)C1CCCCC1]

To a solution of crude (R)-N^{l}-(5-bromo-pyridin-3-yl)-1-phenyl-ethane-1,2-diamine (113 mg, 0.33 mmol) and NN-diisopropylethylamine (85 mg, 0.66 mmol) in dichloromethane (2 mL) was added cyclohexanecarbonyl chloride (48 mg, 0.33 mmol) dropwise at 0° C. The resulting mixture was stirred at room temperature for 2 hours. The reaction mixture was diluted with water (5 mL), and then extracted with dichloromethane (20 mL×3). The combined organic layers were washed with water (5 mL) and brine (5 mL), then dried over anhydrous sodium sulfate, concentrated in vacuo to give cyclohexanecarboxylic acid [(R)-2-(5-bromo-pyridin-3-ylamino)-2-phenyl-ethyl]-amide (147 mg) as yellow oil.

### Step 2: Preparation of cyclohexanecarboxylic acid {(R)-2-[5-(3-methyl-1H-indazol-5-yl)-pyridin-3-ylamino]-2-phenyl-ethyl}-amide

5-Bromo-3-methyl-1H-indazole (63 mg, 0.3 mmol), bis(pinacolato)diboron (78 mg, 0.306 mmol), tris(dibenzylideneacetone)dipalladium (8.2 mg, 0.009 mmol), butyldi-1-adamantylphosphine (9.7 mg, 0.027 mmol), potassium acetate (88 mg, 0.9 mmol) were added into a 10 mL microwave vial containing a magnetic stirrer bar, followed by DME (1 mL). The vessel was sealed with a cap under an argon atmosphere, and then the resulting mixture was heated to 83° C. for 1 hour. Then cyclohexanecarboxylic acid [(R)-2-(5-bromo-pyridin-3-ylamino)-2-phenyl-ethyl]-amide (147 mg, crude), potassium carbonate (124 mg, 0.9 mmol), 1,4-dioxane (2 mL) and H₂O (0.6 mL) were added into the reaction mixture successively. The vessel was sealed with a cap under an argon atmosphere, and then the reaction mixture was heated to 100° C. for 2 hours under microwave. The mixture was cooled to room temperature and diluted with water (5 mL), extracted with ethyl acetate (10 mL×3). The combined organic layers were washed with brine (10 mL), dried over anhydrous sodium sulfate, concentrated in vacuo to give crude title compound. The crude title compound was purified by C-18 reversed phase HPLC column to give cyclohexanecarboxylic acid {(R)-2-[5-(3-methyl-1H-indazol-5-yl)-pyridin-3-ylamino]-2-phenyl-ethyl}-amide (15 mg) as a white solid.

### Example 58

### Preparation of N-{(R)-2-[5-(3-methyl-1H-indazol-5-yl)-pyridin-3-ylamino]-2-phenyl-ethyl}-benzamide

### Step 1: Preparation of N-[(R)-2-(5-bromo-pyridin-3-ylamino)-2-phenyl-ethyl]-benzamide

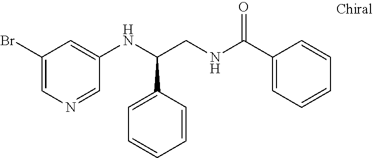 [SMILES: O=C(NC[C@H](Nc1cncc(Br)c1)c1ccccc1)c1ccccc1]

To a solution of crude (R)-N^{l}-(5-bromo-pyridin-3-yl)-1-phenyl-ethane-1,2-diamine (113 mg, 0.33 mmol) and N,N-diisopropylethylamine (85 mg, 0.66 mmol) in dichloromethane (2 mL) was added benzoyl chloride (46 mg, 0.33 mmol) dropwise at 0° C. The resulting mixture was stirred at room temperature for 2 hours. The reaction mixture was diluted with water (5 mL), and then extracted with dichloromethane (20 mL×3). The combined organic layers were washed with water (5 mL) and brine (5 mL), then dried over anhydrous sodium sulfate, concentrated in vacuo to give N-[(R)-2-(5-bromo-pyridin-3-ylamino)-2-phenyl-ethyl]-benzamide (135 mg) as yellow oil which was directly used for the next step without purification.

### Step 2: Preparation of N-{(R)-2-[5-(3-methyl-1H-indazol-5-yl)-pyridin-3-ylamino]-2-phenyl-ethyl}-benzamide

5-Bromo-3-methyl-1H-indazole (63 mg, 0.3 mmol), bis(pinacolato)diboron (78 mg, 0.306 mmol), tris(dibenzylideneacetone)dipalladium (8.2 mg, 0.009 mmol), butyldi-1-adamantylphosphine (9.7 mg, 0.027 mmol), potassium acetate (88 mg, 0.9 mmol) were added into a 10 mL microwave vial containing a magnetic stirrer bar, followed by DME (1 mL). The vessel was sealed with a cap under an argon atmosphere, and then the resulting mixture was heated to 83° C. for 1 hour. Then N-[(R)-2-(5-bromo-pyridin-3-ylamino)-2-phenyl-ethyl]-benzamide (135 mg, crude), potassium carbonate (124 mg, 0.9 mmol), 1,4-dioxane (2 mL) and H₂O (0.6 mL) were added into the reaction mixture successively. The vessel was sealed with a cap under an argon atmosphere. The reaction mixture was heated to 100° C. for 2 hours under microwave. The mixture was cooled to room temperature and diluted with water (5 mL), extracted with ethyl acetate (10 mL×3). The combined organic layers were washed with brine (10 mL), dried over anhydrous sodium sulfate, concentrated in vacuo to give crude title compound. The crude title compound was purified by C-18 reversed phase HPLC column to give N-{(R)-2-[5-(3-methyl-1H-indazol-5-yl)-pyridin-3-ylamino]-2-phenyl-ethyl}-benzamide (27 mg) as a yellow solid.

### Example 59

### Preparation of (R)-2-[5-(3-chloro-1H-indazol-5-yl)-pyridin-3-ylamino]-2-phenyl-acetamide

Under an Ar atmosphere, a mixture of 5-(2-hydroxy-1-phenylethyl)-amino)-pyridin-3-yl)-boronic acid (350 mg, 1.26 mmol), 3-chloro-5-(4,4,5,5-tetramethyl-[1,3,2]dioxaborolan-2-yl)-1H-indazole (371 mg, 1.26 mmol), tetrakis(triphenylphosphine)palladium (70 mg) and potassium carbonate (350 mg, 2.52 mmol) in DME/H₂O (5:1, 15 mL) was heated at 90° C. under microwave for 60 mins. Then the residue was partitioned between EtOAc and brine. The aqueous layer was separated and extracted with EtOAc. The combined organic layers were concentrated and the residue was purified by column chromatography to give 250 mg of 2-[5-(3,3-dimethyl-2-oxo-2,3-dihydro-1H-indol-5-yl)-pyridin-3-ylamino]-2-phenyl-acetamide.

Chiral separation of 2-[5-(3,3-dimethyl-2-oxo-2,3-dihydro-1H-indol-5-yl)-pyridin-3-ylamino]-2-phenyl-acetamide gave (R)-2-[5-(3-chloro-1H-indazol-5-yl)-pyridin-3-ylamino]-2-phenyl-acetamide (65 mg).

### Example 60

### Preparation of (S)-2-[5-(3-chloro-1H-indazol-5-yl)-pyridin-3-ylamino]-2-phenyl-acetamide

Chiral separation of 2-[5-(3,3-dimethyl-2-oxo-2,3-dihydro-1H-indol-5-yl)-pyridin-3-ylamino]-2-phenyl-acetamide gave (S)-2-[5-(3-chloro-1H-indazol-5-yl)-pyridin-3-ylamino]-2-phenyl-acetamide (53 mg).

### BIOLOGICAL EXAMPLES

### Example 61

### CDK8/Cyclin C LANCE TR-FRET Kinase Assay:

The biological activity of the compounds of the invention can be determined using the assay described below.

CDK8/Cyclin C protein was obtained from Invitrogen, cat #PV4402. ULight-Glycogen Synthase (Ulight-GS) peptide with sequence PASVPPSPSLSRHSSPHQ(pS)ED, and Europium-anti-phospho Glycogen Synthase (Ser641) [Eu-anti-P-GS (Ser641)] were obtained from Perkin Elmer, cat #TRF0131-M and cat #TRF0220. Adenosine-5′-triphosphate (ATP) was obtained from Invitrogen, cat #PV3227.

A mixture of (1) a compound of formula I, (2) substrate [Ulight-GS peptide (80 nM) and ATP (24 μM)], and (3) CDK8/Cyclin C (10 nM) in reaction buffer (50 mM Hepes, pH7.0, 10 mM MgCl₂, 1 mM EGTA, 0.2 mg/mL BSA, 0.8 mM DTT) were incubated at 37° C. for 30 mins. Then, [Eu-anti-P-GS (Ser641)] (1.5 nM) was added. Following incubation at room temperature for 30 mins, the TR-FRET signals were detected using Envision reader (Ex 340 nm, Em 615 nm and 665 nm) from Perkin Elmer. The reactivity in percentage of inhibition or dose response was analyzed with GraphPad Prism 5 (GraphPad Software).

Results of CDK8/Cyclin LANCE Ultra biochemical TR-FRET kinase assay are given in Table 1.

### Example 62

### In Vitro Cell Proliferation Assay:

Cells were seeded on 96-well plates at 5×10³ cells per well and precultured for 24 hours. The cells were treated with serial diluted compounds and cultured for 72 hours. Then all media was discarded and after that, 100 μL 1:10 (v/v) Cell Counting Kit-8 (CCK-8)-culture media solution was added to the wells. Plate was developed for 2 hours in an incubator, and the absorbance was measured at 450 nm wavelengths with SpectraMAX190 (MDS, Sunnyvale, Calif.). The inhibition rate (IR) of the tested compounds was determined with following formula: IR (%)=(OD_{DMSO}−ODcompound)/OD_{DMSO}×100%. The concentration corresponding to 50% IR (IC₅₀) was determined with plot curve of IR against tested compound concentrations with SoftMax Pro.

Results of in vitro cell proliferation assay are given in Table 3.

The compounds of the present invention were tested for their capacity to inhibit a CDK8 activity and activation as described herein. The Examples were tested in the above assay and found to have IC₅₀ of about 0.0001 μM to about 30 μM. Particular compounds of formula I were found to have IC₅₀ of about 0.0001 μM to about 1 μM.

### Example A

A compound of formula I can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B

A compound of formula I can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

1. Compounds of formula (I) 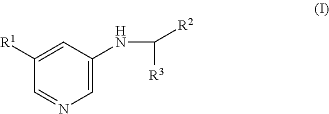
wherein
R¹ is selected from 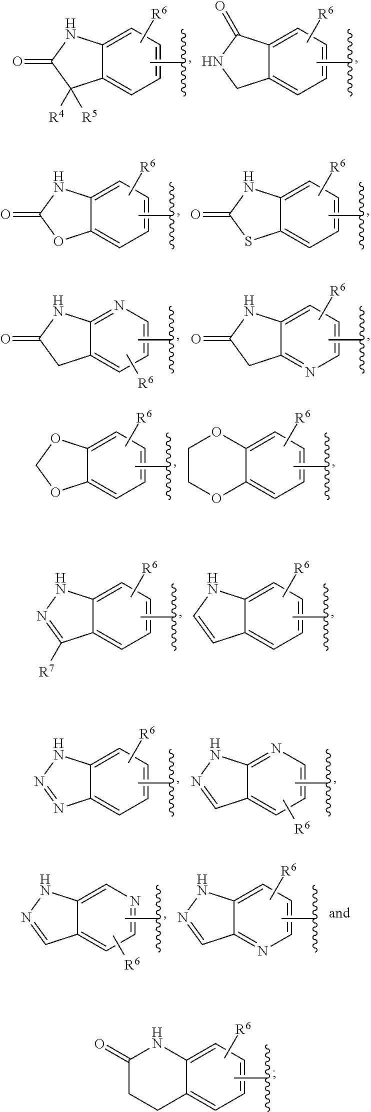
R² is aminocarbonyl, C₁₋₆alkoxy-C_{y}H_{2y}-amino-CₓH₂ₓ—, C₁₋₆alkoxy-CₓH₂ₓ-sulfonylamino-CₓH₂ₓ—, C₁₋₆alkylcarbonylamino-CₓH₂ₓ—, C₁₋₆alkylsulfonylamino-CₓH₂ₓ—, cycloalkylcarbonylamino-CₓH₂ₓ—, cycloalkylsulfonylamino-CₓH₂ₓ—, hydroxy-CₓH₂ₓ—, hydroxy-C_{y}H_{2y}-amino-CₓH₂ₓ—, hydroxy-CₓH₂ₓ-carbonylamino-CₓH₂ₓ— or phenylcarbonylamino-CₓH₂ₓ—;
R³ is phenyl, which is unsubstituted or substituted by halogen;
R⁴ is hydrogen, C₁₋₆alkyl or halogen;
R⁵ is hydrogen, C₁₋₆alkyl or halogen;
or R⁴ and R⁵, together with the carbon atom, to which they are attached, form cycloalkyl;
R⁶ is hydrogen or halogen;
R⁷ is hydrogen, C₁₋₆alkyl, C₁₋₆alkylsulfanyl, C₁₋₆alkylsulfonyl, amino or halogen;
x is 1-6;
y is 2-6;
or pharmaceutically acceptable salt thereof.

2. A compound according to claim 1, wherein
R¹ is selected from 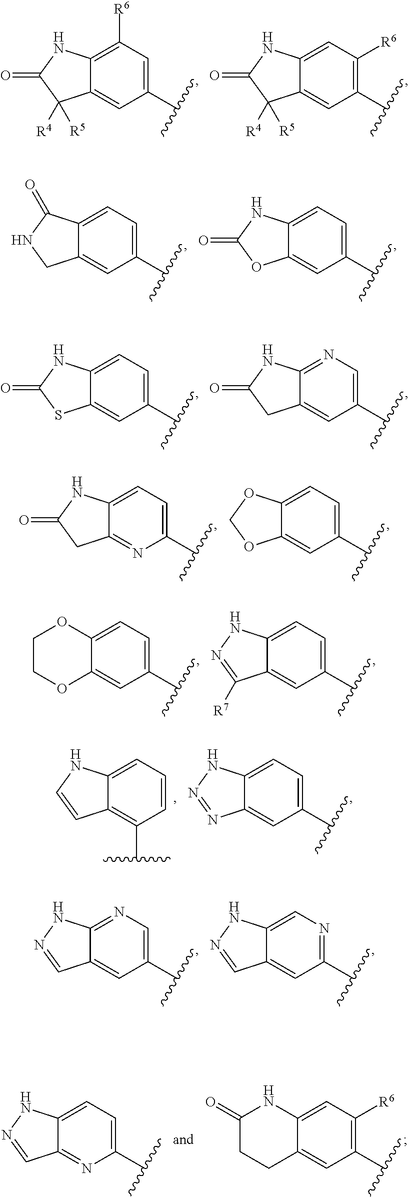
R² is aminocarbonyl, C₁₋₆alkoxy-C_{y}H_{2y}-amino-CₓH₂ₓ—, C₁₋₆alkoxy-CₓH₂ₓ-sulfonylamino-CₓH₂ₓ—, C₁₋₆alkylcarbonylamino-CₓH₂ₓ—, C₁₋₆alkylsulfonylamino-CₓH₂ₓ—, cycloalkylcarbonylamino-CₓH₂ₓ—, cycloalkylsulfonylamino-CₓH₂ₓ—, hydroxy-CₓH₂ₓ—, hydroxy-C_{y}H_{2y}-amino-CₓH₂ₓ—, hydroxy-CₓH₂ₓ-carbonylamino-CₓH₂ₓ— or phenylcarbonylamino-CₓH₂ₓ—;
R³ is phenyl, which is unsubstituted or once substituted by halogen;
R⁴ is hydrogen, C₁₋₆alkyl or halogen;
R⁵ is hydrogen, C₁₋₆alkyl or halogen;
or R⁴ and R⁵, together with the carbon atom, to which they are attached, form cycloalkyl;
R⁶ is hydrogen or halogen;
R⁷ is hydrogen, C₁₋₆alkyl, C₁₋₆alkylsulfanyl, C₁₋₆alkylsulfonyl, amino or halogen;
x is 1-6;
y is 2-6;
or pharmaceutically acceptable salt thereof.

3. A compound according to claim 1, wherein
R¹ is selected from 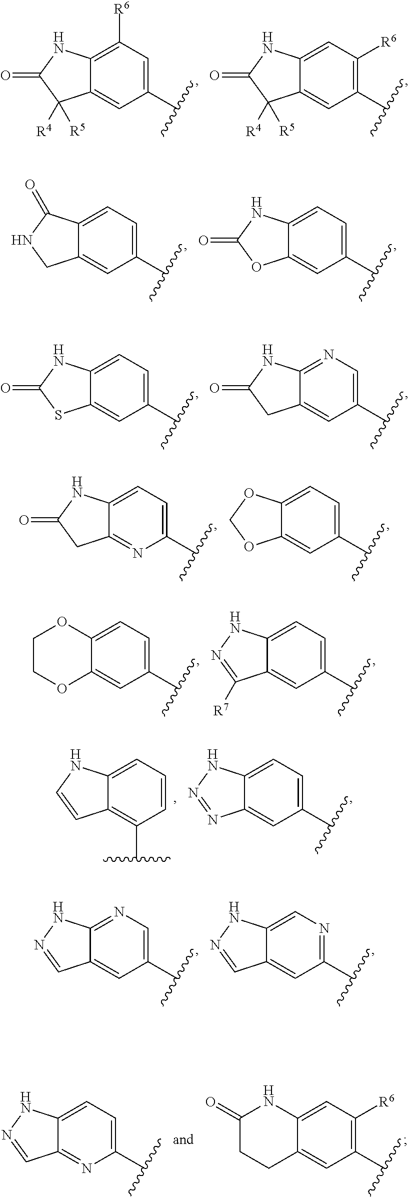
R² is aminocarbonyl, methoxyethylaminomethyl, methoxyethylsulfonylaminomethyl, methylcarbonylaminomethyl, ethylcarbonylaminomethyl, isopropylcarbonylaminomethyl, methylsulfonylaminomethyl, cyclohexylcarbonylaminomethyl, cyclopropylsulfonylaminomethyl, hydroxymethyl, hydroxyethylaminomethyl, hydroxymethylcarbonylaminomethyl or phenylcarbonylaminomethyl;
R³ is phenyl or chlorophenyl;
R⁴ is hydrogen, methyl or fluoro;
R⁵ is hydrogen, methyl or fluoro;
or R⁴ and R⁵, together with the carbon atom, to which they are attached, form cyclopropyl;
R⁶ is hydrogen or fluoro;
R⁷ is hydrogen, methyl, ethyl, methylsulfanyl, methylsulfonyl, amino, fluoro or chloro;
or pharmaceutically acceptable salt thereof.

4. A compound according to claim 1, wherein
R¹ is 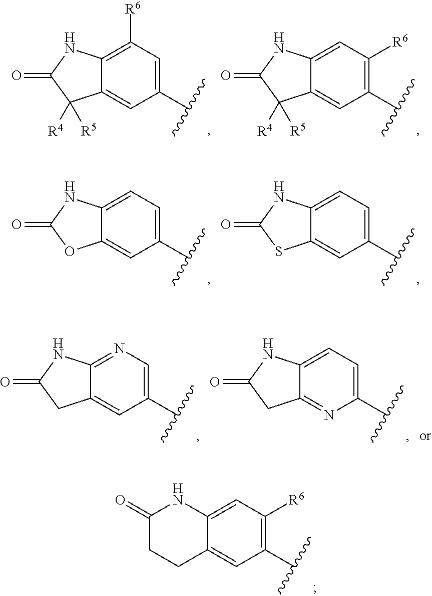
R² is aminocarbonyl, C₁₋₆alkylcarbonylamino-CₓH₂ₓ— or hydroxy-CₓH₂ₓ—;
R³ is phenyl, which is unsubstituted or once substituted by halogen;
R⁴ is hydrogen, C₁₋₆alkyl or halogen;
R⁵ is hydrogen, C₁₋₆alkyl or halogen;
or R⁴ and R⁵, together with the carbon atom, to which they are attached, form cycloalkyl;
R⁶ is hydrogen or halogen;
x is 1-6.

5. A compound according to claim 4, wherein
R¹ is 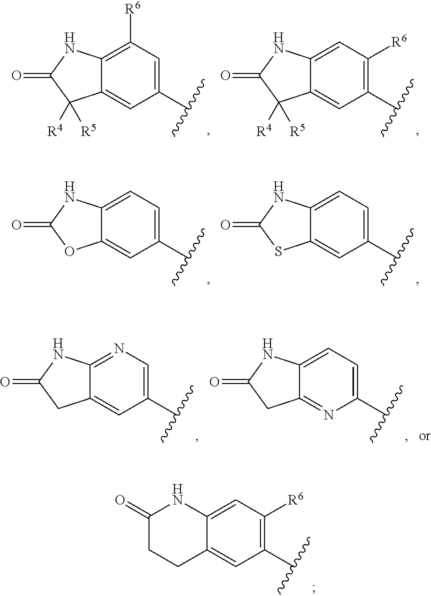
R² is aminocarbonyl, methylcarbonylaminomethyl or hydroxymethyl;
R³ is phenyl or chlorophenyl;
R⁴ is hydrogen, methyl or fluoro;
R⁵ is hydrogen, methyl or fluoro;
or R⁴ and R⁵, together with the carbon atom, to which they are attached, form cyclopropyl;
R⁶ is hydrogen or fluoro.

6. A compound according to claim 1, wherein
R¹ is 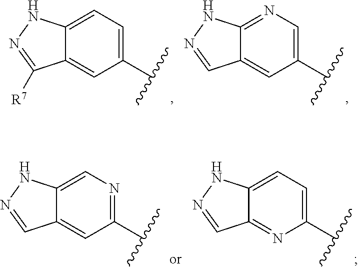
R² is aminocarbonyl, C₁₋₆alkoxy-C_{y}H_{2y}-amino-CₓH₂ₓ—, C₁₋₆alkoxy-CₓH₂ₓ-sulfonylamino-CₓH₂ₓ—, C₁₋₆alkylcarbonylamino-CₓH₂ₓ—, C₁₋₆ alkylsulfonylamino-CₓH₂ₓ—, cycloalkylcarbonylamino-CₓH₂ₓ—, cycloalkylsulfonylamino-CₓH₂ₓ—, hydroxy-CₓH₂ₓ—, hydroxy-C_{y}H_{2y}-amino-CₓH₂ₓ—, hydroxy-CₓH₂ₓ-carbonylamino-CₓH₂ₓ— or phenylcarbonylamino-CₓH₂ₓ—;
R³ is phenyl;
R⁷ is hydrogen, C₁₋₆alkyl, C₁₋₆alkylsulfanyl, C₁₋₆alkylsulfonyl, amino or halogen;
x is 1-6;
y is 2-6.

7. A compound according to claim 6, wherein
R¹ is 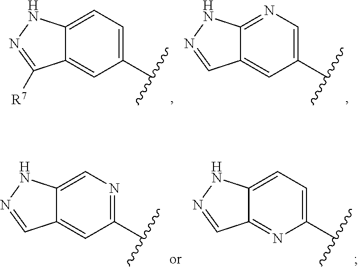
R² is aminocarbonyl, methoxyethylaminomethyl, methoxyethylsulfonylaminomethyl, methylcarbonylaminomethyl, ethylcarbonylaminomethyl, isopropylcarbonylaminomethyl, methylsulfonylaminomethyl, cyclohexylcarbonylaminomethyl, cyclopropylsulfonylaminomethyl, hydroxymethyl, hydroxyethylaminomethyl, hydroxymethylcarbonylaminomethyl or phenylcarbonylaminomethyl;
R³ is phenyl;
R⁷ is hydrogen, methyl, ethyl, methylsulfanyl, methylsulfonyl, amino, fluoro or chloro.

8. A compound according to claim 1, selected from
5-[5-((R)-2-Hydroxy-1-phenyl-ethylamino)-pyridin-3-yl]-1,3-dihydro-indol-2-one;
5-[5-((R)-2-Hydroxy-1-phenyl-ethylamino)-pyridin-3-yl]-3,3-dimethyl-1,3-dihydro-indol-2-one;
5-{5-[(R)-1-(2-Chloro-phenyl)-2-hydroxy-ethylamino]-pyridin-3-yl}-1,3-dihydro-indol-2-one;
5-{5-[(S)-1-(2-Chloro-phenyl)-2-hydroxy-ethylamino]-pyridin-3-yl}-1,3-dihydro-indol-2-one;
5-[5-((R)-2-Hydroxy-1-phenyl-ethylamino)-pyridin-3-yl]-1,3-dihydro-benzoimidazol-2-one;
(R)-5′-(5-((2-hydroxy-1-phenylethyl)amino)pyridin-3-yl)-spiro[cyclopropane-1,3′-indolin]-2′-one;
5-[5-((R)-2-Hydroxy-1-phenyl-ethylamino)-pyridin-3-yl]-1,3-dihydro-pyrrolo[2,3-b]pyridin-2-one;
3,3-Difluoro-5-[5-((R)-2-hydroxy-1-phenyl-ethylamino)-pyridin-3-yl]-1,3-dihydro-indol-2-one;
5-[5-((R)-2-Hydroxy-1-phenyl-ethylamino)-pyridin-3-yl]-1,3-dihydro-pyrrolo[3,2-b]pyridin-2-one;
6-Fluoro-5-[5-((R)-2-hydroxy-1-phenyl-ethylamino)-pyridin-3-yl]-1,3-dihydro-indol-2-one;
7-Fluoro-5-[5-((R)-2-hydroxy-1-phenyl-ethylamino)-pyridin-3-yl]-1,3-dihydro-indol-2-one;
6-[5-((R)-2-Hydroxy-1-phenyl-ethylamino)-pyridin-3-yl]-3H-benzooxazol-2-one;
6-[5-((R)-2-Hydroxy-1-phenyl-ethylamino)-pyridin-3-yl]-3H-benzothiazol-2-one;
(R)-2-(5-Benzo[1,3 ]dioxol-5-yl-pyridin-3-ylamino)-2-phenyl-ethanol;
(R)-2-[5-(2,3-Dihydro-benzo[1,4]dioxin-6-yl)-pyridin-3-ylamino]-2-phenyl-ethanol;
(R)-2-[5-(1H-Indazol-5-yl)-pyridin-3-ylamino]-2-phenyl-ethanol;
(R)-2-[5-(1H-Indol-4-yl)-pyridin-3-ylamino]-2-phenyl-ethanol;
(R)-2-[5-(3-Amino-1H-indazol-5-yl)-pyridin-3-ylamino]-2-phenyl-ethanol;
(R)-2-[5-(3-Fluoro-1H-indazol-5-yl-pyridin-3-ylamino]-2-phenyl-ethanol;
(R)-2-[5-(3-Methyl-1H-indazol-5-yl)-pyridin-3-ylamino]-2-phenyl-ethanol;
(R)-2-[5-(3-Ethyl-1H-indazol-5 -yl)-pyridin-3-ylamino]-2-phenyl-ethanol;
(R)-2-[5-(3-Methylsulfanyl-1H-indazol-5-yl)-pyridin-3-ylamino]-2-phenyl-ethanol;
(R)-2-[5-(3-Methanesulfonyl-1H-indazol-5-yl)-pyridin-3-ylamino]-2-phenyl-ethanol;
(R)-2-Phenyl-2-[5-(1H-pyrazolo[3,4-b]pyridin-5-yl)-pyridin-3-ylamino]-ethanol;
(R)-2-Phenyl-2-[5-(1H-pyrazolo[3,4-c]pyridin-5-yl)-pyridin-3-ylamino]-ethanol;
(R)-2-[5-(1H-Benzotriazol-5-yl)-pyridin-3-ylamino]-2-phenyl-ethanol;
(R)-2-Phenyl-2-[5-(1H-pyrazolo[4,3-b]pyridin-5-yl)-pyridin-3-ylamino]-ethanol;
(R)-2-[5-(3-Chloro-1H-indazol-5-yl)-pyridin-3-ylamino]-2-phenyl-ethanol;
2-[5-(2-Oxo-2,3-dihydro-1H-indol-5-yl)-pyridin-3-ylamino]-2-phenyl-acetamide;
N-{(R)-2-[5-(2-Oxo-2,3-dihydro-1H-indol-5-yl)-pyridin-3-ylamino]-2-phenyl-ethyl}-acetamide;
2-[5-(1-Oxo-2,3-dihydro-1H-isoindol-5-yl)-pyridin-3-ylamino]-2-phenyl-acetamide;
2-[5-(3,3-Dimethyl-2-oxo-2,3-dihydro-1H-indol-5-yl)-pyridin-3-ylamino]-2-phenyl-acetamide;
2-[5-(2-Oxo-1,2,3,4-tetrahydro-quinolin-6-yl)-pyridin-3-ylamino]-2-phenyl-acetamide;
2-[5-(7-Fluoro-2-oxo-1,2,3,4-tetrahydro-quinolin-6-yl)-pyridin-3-ylamino]-2-phenyl-acetamide;
2-(5-Benzo[1,3]dioxol-5-yl-pyridin-3-ylamino)-2-phenyl-acetamide;
2-[5 -(2,3-Dihydro-benzo[1,4]dioxin-6-yl)-pyridin-3-ylamino]-2-phenyl-acetamide;
2-[5-(1H-Indazol-5-yl)-pyridin-3-ylamino]-2-phenyl-acetamide;
(R)-2-[5-(1H-Indazol-5-yl)-pyridin-3-ylamino]-2-phenyl-acetamide;
(S)-2-[5-(1H-indazol-5-yl)-pyridin-3-ylamino]-2-phenyl-acetamide;
N-{(R)-2-[5-(1H-Indazol-5-yl)-pyridin-3-ylamino]-2-phenyl-ethyl}-acetamide;
N-{(R)-2-[5-(1H-Indazol-5-yl)-pyridin-3-ylamino]-2-phenyl-ethyl}-methanesulfonamide;
2-[5-(3-Fluoro-1H-indazol-5-yl)-pyridin-3-ylamino]-2-phenyl-acetamide;
N-{(R)-2-[5-(3-Fluoro-1H-indazol-5-yl)-pyridin-3-ylamino]-2-phenyl-ethyl}-propionamide;
N-{(R)-2-[5-(3-Fluoro-1H-indazol-5-yl)-pyridin-3-ylamino]-2-phenyl-ethyl}-isobutyramide;
(R)-N^{l}-[5-(3-Fluoro-1H-indazol-5-yl)-pyridin-3-yl]-N²-(2-methoxy-ethyl)-1-phenyl-ethane-1,2-diamine;
N-{(R)-2-[5-(3-Fluoro-1H-indazol-5-yl)-pyridin-3-ylamino]-2-phenyl-ethyl}-2-hydroxy-acetamide;
2-{(R)-2-[5-(3-Fluoro-1H-indazol-5-yl)-pyridin-3-ylamino]-2-phenyl-ethylamino}-ethanol;
(R)-2-[5-(3-Methyl-1H-indazol-5-yl)-pyridin-3-ylamino]-2-phenyl-acetamide;
(S)-2-[5-(3-Methyl-1H-indazol-5-yl)-pyridin-3-ylamino]-2-phenyl-acetamide;
N-{2-[5-(3-Methyl-1H-indazol-5-yl)-pyridin-3-ylamino]-2-phenyl-ethyl}-methanesulfonamide;
Cyclopropanesulfonic acid {(R)-2-[5-(3-methyl-1H-indazol-5-yl)-pyridin-3-ylamino]-2-phenyl-ethyl}-amide;
N-{(R)-2-[5-(3-Methyl-1H-indazol-5-yl)-pyridin-3-ylamino]-2-phenyl-ethyl}-methanesulfonamide;
2-Methoxy-ethanesulfonic acid {(R)-2-[5-(3-methyl-1H-indazol-5-yl)-pyridin-3-ylamino]-2-phenyl-ethyl}-amide;
2-Hydroxy-N-{(R)-2-[5-(3-methyl-1H-indazol-5-yl)-pyridin-3-ylamino]-2-phenyl-ethyl}-acetamide;
(R)-N²-(2-Methoxy-ethyl)-N^{l}-[5-(3-methyl-1H-indazol-5-yl)-pyridin-3-yl]-1-phenyl-ethane-1,2-diamine;
2-{(R)-2-[5-(3-Methyl-1H-indazol-5-yl)-pyridin-3-ylamino]-2-phenyl-ethylamino}-ethanol;
Cyclohexanecarboxylic acid {(R)-2-[5-(3-methyl-1H-indazol-5-yl)-pyridin-3-ylamino]-2-phenyl-ethyl}-amide;
N-{(R)-2-[5-(3-Methyl-1H-indazol-5-yl)-pyridin-3-ylamino]-2-phenyl-ethyl}-benzamide;
(R)-2-[5-(3-Chloro-1H-indazol-5-yl)-pyridin-3-ylamino]-2-phenyl-acetamide; and
(S)-2-[5-(3-Chloro-1H-indazol-5-yl)-pyridin-3-ylamino]-2-phenyl-acetamide.

9. A process for the preparation of a compound of claim 1, comprising the reaction of
(a) a compound of formula (A) 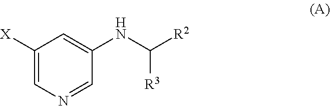
with 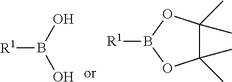
in the presence of a catalyst and a base;
(b) a compound of formula (B) 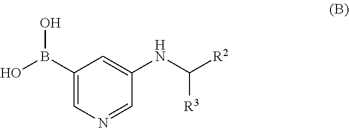
with R¹—X in the presence of a catalyst and a base;
(c) a compound of formula (A) 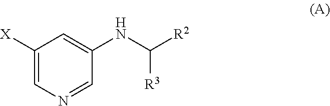
with bis(pinacolato)diboron and R¹—X in the presence of a catalyst and a ligand under microwave;
(d) a compound of formula (C) 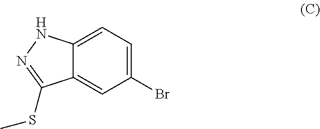
in the presence of a catalyst;
(e) a compound of formula (D) 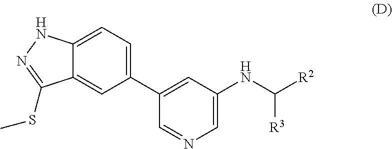
in the presence of oxone;
(f) a compound of formula (E) 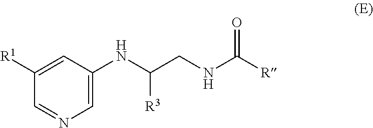
in the presence of BH₃;
wherein R¹, R² and R³ are defined as in any one of claims 1 to 9; X is chloro, bromo or iodo; R″ is C₁₋₆alkyl or C₁₋₆alkoxy-CH₂—.

10. A compound according to claim 1 for use as therapeutically active substance.

11. A pharmaceutical composition comprising a compound in accordance with claim 1 and a therapeutically inert carrier.

12. A method for the treatment of cancer, the method comprising administering an effective amount of a compound according to claim 1 to a subject in need thereof.

13. The method of claim 12, wherein the cancer is selected from the group consisting of bladder, head and neck, breast, stomach, ovary, colon, lung, brain, larynx, lymphatic system, liver, skin, hematopoetic system, genitourinary tract, gastrointestinal, ovarian, prostate, gastric, bone, small-cell lung, glioma, colorectal and pancreatic cancers.

14. The method of claim 12, wherein the cancer is gastric cancer or colorectal cancer.

15. A compound according to claim 1, wherein the compound is an inhibitor of CDK8 or Cyclin C.

16. A compound according to claim 1, when manufactured according to a process of claim 9.
